(19) United States
(12) Patent Application Publication
(10) Pub. No.: US 2024/0287571 A1
(43) Pub. Date: Aug. 29, 2024

(54) METHODS AND KITS FOR DETECTING CROSS-CONTAMINATION IN FOODBORNE AND ENVIRONMENTAL PATHOGEN TESTING

(71) Applicant: Applied Food Diagnostics, Inc., Bloomsburg, PA (US)

(72) Inventors: Thomas Lonczynski, Bloomsburg, PA (US); Laura Cowin, Bloomsburg, PA (US)

(21) Appl. No.: 18/176,270

(22) Filed: Feb. 28, 2023

(51) Int. Cl. C12Q 1/04; C12Q 1/686

(52) U.S. Cl. CPC C12Q 1/04; C12Q 1/686

(57) ABSTRACT

The present disclosure is directed to a method for simultaneously detecting presence or absence of a target bacteria and a reporter labeled protein in a sample to determine the occurrence of cross-contamination, and kits and composition for carrying out such methods.

### FIELD

The present disclosure is directed to methods for simultaneously detecting presence or absence of a target bacteria alongside a reporter labeled quality control strain of the target bacteria in a sample to detect potential cross-contamination, and kits and compositions for carrying out such methods.

### SEQUENCE LISTING

The Sequence Listing is being submitted electronically in XML format and is hereby incorporated by reference in its entirety. Said XML copy, created on Feb. 21, 2023, is named 263280_000100_ST26.xml and is 63,728 bytes in Size.

### BACKGROUND

Foodborne diseases can be severe and are the most common illnesses resulting from unsafe food, especially for young children. Salmonella is the most common bacteria to cause food poisoning in the United States. Tens of millions of cases are reported every year worldwide. While most people get better without treatment within a few days, some cases can lead to hospitalization or even death. The most common food sources for the illness are raw or undercooked meat, raw fruits and vegetables, unpasteurized milk or other dairy products, raw or undercooked eggs, and processed foods such as chicken nuggets and nut butters.

One of the most serious and severe foodborne diseases is caused by Listeria species due to the high rate of death associated with this infection, making it a significant public health concern. Listeria, specifically Listeria monocytogenes, causes Listeriosis, a serious illness caused by eating food contaminated by the bacterium. It is estimated that 1,600 people get sick with the illness each year, and 260 deaths are reported. Listeriosis most commonly affects pregnant women, newborns, the elderly, and people with weakened immune systems. Since L. monocytogenes can survive and multiply at low temperatures, foods involved in Listeria infection can include meat and dairy products, as well as fresh vegetables and fruits.

E. coli bacteria are a diverse group of bacteria that are usually harmless. However, there are some pathogenic forms of the bacteria, one of which produces a toxin called Shiga-toxin. This form of bacteria is known as Shiga-toxin producing E. coli (STEC). The most common and widely known STEC related to foodborne illnesses and outbreaks is E. coli O157:H7. The other serogroups classified as part of the “Top 7” are O26, O45, O103, O111, O121, and O145, as these along with O157:H7 are declared as adulterants in beef by the U.S. Department of Agriculture (USDA) Food Safety and Inspection Service (FSIS). Sources of contamination often include raw or undercooked ground meat products, salami, unpasteurized milk and fruit juice, yogurt, cheese, raw vegetables, and contamination during food preparation from contaminated surfaces.

In addition to foodborne and environmental contamination from pathogens, laboratories must also concern themselves with cross-contamination, which is the unintentional transfer of microorganisms from one object or person to another. If not diligent, samples can be cross-contaminated with a quality control strain during analysis. Cross-contamination can cause numerous issues for a laboratory, leading to operational shutdowns, cleaning, delays in testing, and risks to laboratory employees. Additionally, it can lead to the reporting of false positive pathogen results. Preventing false positives is of paramount importance. A false positive can lead to a recall of products, causing significant financial loss for a company as well as potential harm to consumers.

Methods for detecting contaminating pathogens in food products and environmental surfaces exist but are often difficult to carry out due to complex biotechnology requirements. In addition, detection methods for pathogens as well as ruling out any cross-contamination can be expensive and time-consuming. There is a need in the art for rapid accurate detection methods that provide reliable and accurate results. In addition, there is a need for detection methods capable of simultaneous detection of contaminating pathogens and any potential cross-contamination to prevent false positives.

This disclosure is directed to overcoming these and other deficiencies in the art.

### SUMMARY

One aspect of the present disclosure relates to a method for detecting cross-contamination and the presence or absence of a target bacterial species in a test sample. This method involves providing (i) a test sample potentially comprising a target bacteria and (ii) a control sample, where the control sample comprises a control bacterial strain engineered with a reporter molecule. The method further involves lysing the test sample and the control sample to generate a test sample lysate and a control sample lysate, contacting each of the test sample lysate and the control sample lysate with an assay mixture comprising one or more bacterial detection PCR primer sets and a control detection PCR primer set, thereby generating one or more assay samples capable of amplifying a target, and detecting presence or absence of an amplified target PCR product and/or an amplified control PCR product in each of the one or more assay samples, thereby determining the presence or absence of the target bacterial species and/or the control bacterial strain in the samples simultaneously. The method further involves determining if cross-contamination of the test sample occurred by detecting if the amplified control PCR product is in the test sample.

Another aspect of the present disclosure relates to a kit comprising one or more PCR primer sets capable of detecting Salmonella, Listeria, or E. coli species and one or more PCR primer sets capable of detecting a control bacterial strain engineered with a reporter molecule.

The methods and kits described herein comprise a multifaceted approach to the detection of bacterial species, including Salmonella, Listeria, and E. coli species in a variety of food products and environmental samples. A specifically formulated media can be utilized for selectively enriching samples followed by cultural (detection plate) and rapid (qPCR) detection procedures. During PCR amplification, forward and reverse primers hybridize to unique sequences of bacterial genomic DNA. A fluorogenic probe can be included in the same reaction mixture, which includes a DNA probe labeled with a 5′-dye and a 3′-quencher. During PCR amplification, the probe is cleaved and the reporter dye and quencher are separated. The resulting increase in fluorescence can be detected on a real-time PCR instrument. The combination of the multiple detection approaches ensures cost-effectiveness and efficiency. Inclusion of a control bacterial strain tagged with Green Fluorescent Protein (“GFP”), and corresponding detection in each multiplex assay limits time and labor in investigations into cross-contamination issues and allows distinction of positive results from cross-contamination, preventing false positives.

Reporter labeled quality control strains, stuch as strains tagged with GFP, are microogranisms that are used as a benchmark to ensure the laboratory's testing methods are accurate and reliable. Currently, if a laboratory wants to verify a positive result was not caused by cross-contamination, the enriched sample needs to be streaked to a plate and incubated for 24-72 hours, causing a lengthy delay. After the enrichment time, the growth on the plate would need to be examined under UV light for fluorescence of the GFP molecule. The delay in results (24-72 hours) can be critical for a food manufacturer, especially when product is on hold and shelf lives of products can be short to begin with. Additionally, fluorescence can sometimes be difficult to see, leading to questions on whether the positive was a cross-contamination issue or not.

With the methods and systems of the present disclosure, the GFP is detected at the same time as the target organism in the same PCR run, meaning that if the GFP target amplified in a sample that was not the quality control strain, cross-contamination had occurred. Thus, the methods and systems of the present disclosure make it possible to know if there is a cross-contamination event immediately, and not 24-72 hours after the initial run. In addition to time savings, no additional materials are needed for further testing. Results are not delayed preventing potential impacts on delayed shipment of potentially contaminated samples due to potential false positive results.

While simultaneous detection of the pathogen of interest and GFP target was found to be important, it was also determined the timing of the PCR detection for both the target pathogen and GFP was critical. Specifically, the Cq value, or threshold cycle, indicates the PCR cycle that the DNA of the target was detected by the PCR reaction. Typically, the larger amount of DNA present in sample, the lower the Cq value. Thus, if there were low levels of target DNA of the pathogen present, the DNA would amplify at a much later cycle in the PCR process. If the amplification of the target for the pathogen of interest was detected at a much lower Cq value than the GFP target, it was possible the target for the GFP would not be detectable. This would prevent detection of cross-contamination. If the GFP target amplified a few cycles after the target pathogen, the amplification could occur too late and the GFP target would not be detected in time. To overcome this issue, the methods and systems of the present disclosure were developed such that the Cq of the GFP target occurs at about the same time or earlier than the pathogen of interest. This feature was found to be paramount in preventing false positives.

### BRIEF DESCRIPTION OF THE DRAWINGS

FIGS. 1A-E illustrate a workflow of one embodiment of an exemplary multiplex PCR reaction for target species detection. FIG. 1A: the workflow begins with one or more test samples and a control sample (e.g., a Salmonella, Listeria, or E. coli strain engineered with GFP). FIG. 1B: the samples are enriched by incubation in enrichment broth (e.g., Buffered Peptone Water (“BPW”)) and/or selective media (e.g., Enterohemorrhagic E. coli Recovery and Enrichment Broth (“EREB”), or Listeria Recovery and Enrichment Broth (“LREB”)). * Incubation times and temperatures may vary depending on matrix. In some instances, enrichment incubation times may lengthen if lysates are pooled. Type of enrichment broth and/or selective media used, incubation time, and incubation temperature can also vary by sample type. See Examples 1-5, infra. FIG. 1C: A portion of the enriched sample is lysed. FIG. 1D: the lysed test sample and lysed control sample are amplified using qPCR and various PCR probe sets. FIG. 1E: the qPCR results are analyzed based on the amplification curve, which indicates if a positive or negative result for the PCR probe set is obtained. If no amplification occurs in the pathogen or GFP target channel, the sample is negative for both. If amplification occurs in the pathogen target channel, the sample is positive for the pathogen. Amplification in the GFP target channel should only occur in the control sample. If amplification in the GFP target channel occurs in a test sample, cross-contamination has occurred.

### DETAILED DESCRIPTION

The present disclosure relates to methods for simultaneous multiplex detection of target pathogens and control strains in a sample, and kits for carrying out these methods.

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, such as within 10% or within 5% of a given value or range.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

A “nucleic acid”, “nucleotide”, or “polynucleotide” is a polymeric compound comprised of covalently linked subunits called nucleotides. Nucleic acids include polyribonucleic acid (“RNA”) and polydeoxyribonucleic acid (“DNA”), both of which may be single-stranded or double-stranded. DNA includes, but is not limited to, cDNA, genomic DNA, plasmid DNA, synthetic DNA, and semi-synthetic DNA. DNA may be linear, circular, or supercoiled.

The term “fragment” when referring to a polynucleotide will be understood to mean a nucleotide sequence of reduced length relative to the reference nucleic acid (e.g., a wild type) and comprising, over the common portion, a nucleotide sequence identical to the reference nucleic acid. Such a nucleic acid fragment according to the present application may be, where appropriate, included in a larger polynucleotide of which it is a constituent. Such fragments comprise, or alternatively consist of, oligonucleotides ranging in length from at least 8, 10, 12, 15, 18, 20 to 25, 30, 40, 50, 70, 80, 100, 200, 500, 1000, 1500, or any number or range therein, consecutive nucleotides of a nucleic acid described herein.

A “gene” refers to an assembly of nucleotides that encode a functional RNA or a polypeptide and includes cDNA and genomic DNA nucleic acids. “Gene” also refers to a nucleic acid fragment that expresses a specific protein or polypeptide, optionally including regulatory sequences preceding (5′ noncoding sequences) and following (3′ noncoding sequences) the coding sequence.

A “foreign” gene or “exogenous” gene refers to a gene not normally found in the host organism, but that is introduced into the host organism by gene transfer. Foreign genes can comprise native genes inserted into a nonnative organism, or chimeric genes.

An “engineered” bacterial strain refers to a bacterial strain that has been genetically modified by the addition of a foreign or exogenous genetic material in the form of DNA or RNA to the total genetic material of the bacterial cell.

One aspect of the present disclosure relates to a method for detecting cross-contamination and the presence or absence of a target bacterial species in a test sample. This method involves providing (i) a test sample potentially comprising a target bacteria and (ii) a control sample, where the control sample comprises a control bacterial strain engineered with a reporter molecule. The method further involves lysing the test sample and the control sample to generate a test sample lysate and a control sample lysate, contacting each of the test sample lysate and the control sample lysate with an assay mixture comprising one or more bacterial detection PCR primer sets and a control detection PCR primer set, thereby generating one or more assay samples capable of amplifying a target, and detecting presence or absence of an amplified target PCR product and/or an amplified control PCR product in each of the one or more assay samples, thereby determining the presence or absence of the target bacterial species and/or the control bacterial strain in the samples simultaneously. The method further involves determining if cross-contamination of the test sample occurred by detecting if the amplified control PCR product is in the test sample.

This aspect of the present disclosure can be carried out with any of the embodiments disclosed herein.

In some embodiments, a test sample potentially comprising a target bacteria is provided. In some embodiments, the target bacteria is a pathogenic bacteria, such as a bacteria from the genus Salmonella, Listeria, Escherichia, Campylobacter, Vibrio, or Cronobacter, or any combination thereof. While these are common pathogenic bacteria that contaminate food sources, other bacteria may also be present and tested for as the target bacteria of the methods and kits of the present disclosure.

In some embodiments, the target bacterial species is from the genus Salmonella. In some embodiments, the Salmonella bacterial species is, for example and without limitation, one or more of Salmonella enterica subspecies enterica, houtenae, diarizonae, salamae, indica, IV, arizonae, salamae, and Salmonella bongori. In some embodiments, the Salmonella serovar is, for example and without limitation, one or more of Typhimurium; 1, 40:g, z51:-; 11:b:e, n, x; 16:z4, z32:-; 38[k]:z35:-; 40:z4, z24:-; 42:f:g,t:-; 45:a:e, n, x; 45a,b:g,z32:-; Agona (B); Anatum (E1); 62:z4, z23:--; 62:z36:--; 51:z(4), z(23):(−); II 17:g,t:[e,n,x,z15]; II 6,7:b:e,n,x:z42; 48:z35:-; 66:z41:--; 48:z41:--; 66:z(41):(−); 66:z41:-; Choleraesuis; Decatur; Derby (B); 6,7:1,v:z(53); Dublin (D1); Bredeney (B); Duisberg; Emek (C3); Newport (C2); Enteritidis (D1); Montevideo (C1); Schwarzengrund (B); Typhi (D1); Typhimurium (B); Poona (G1); Blockley C2); Livingstone (C1); Hvittingfoss (I); Bareilly (C1); Mbandaka (C1); Setubal (60); Ferlac (VI 1,6,14,25); Flint (IV 50); Gallinarum (D1); Haifa (B); Heidelberg (B); 45:g,z(51):(−); Indiana (B); 1,6, 14,25:a:e,n,x; Infantis (C1); Argentina (IV 6,7); Paratyphi B var. Java; Krefeld (E4); Malawi (66); Maregrosso (V 66); Miami (D1); 58:d:z; Muenchen (C2); Naestved (D1); Newport (C2); 50:k:z; Ohio (C1); Panama (D1); Paratyphi A; Paratyphi B; Paratyphi C; Oranienburg (C1); Javiana (D1); Pullorum (D1); Reading (B); Rissen (C1); Rubislaw (11); S.4, 5, 12:i:-; Saintpaul (B); 1,9,12:1,w:e,n,x; Abortusovis (B); Sendai (D1); Senftenberg (E4); 44:z39:--; Stanley (B); Stanleyville (B); Tennessee (C1); Thompson (C1); Hadar (C2); Virchow (C1); Kentucky (C3); Typhisuis (B); Vrindaban (VI 45); Wassenaar (IV 50); Weltevreden (E1); Wien (B); Brandenberg (B); 45:a:e,n,x; 6,7:1, v:z53; 1,9,12, [Vi]:g,p:--; 11:z4, z23:; 9,46:z4z24:z39:z42; 55:k:z39; 1,4, 12,27:b:l, w; IIIb 47:1:z53:z57; 18:z4, z32:; 50:r:z; and 45:g,z51:-.

In further embodiments, the target bacterial species is from the genus Listeria. In some embodiments, the Listeria bacterial species is, for example and without limitation, one or more of Listeria spp., Listeria monocytogenes, Listeria grayi, Listeria ivanovii, Listeria innocua, Listeria welshmeri, Listeria seeligeri, Listeria marthii, Listeria aquatica, and Listeria riparia.

In another embodiment, the target bacterial species is from the genus Escherichia. In some embodiments, the Escherichia bacterial species is, for example and without limitation, a Shiga toxin-producing Escherichia coli. Shiga toxin-producing E. coli produce toxins, known as Shiga-toxins due to their similarity to the toxins produced by Shigella dysenteriae. In some embodiments, the Escherichia bacterial species is, for example and without limitation, one or more of E. coli O157:H7, O26, O45, O103, O111, O121, or O145.

In another embodiment, the target bacterial species is from the genus Campylobacter, Vibrio, or Cronobacter.

In the methods of the present disclosure, a control sample comprising a control bacterial strain is provided. In some embodiments, the control bacterial strain is engineered with a reporter molecule. In some embodiments, the reporter molecule comprises a detectable polypeptide, for example an enzyme, a fluorescent polypeptide, a luminescent polypeptide, or an antigen.

Engineering a bacterial strain with a reporter molecule can be carried out by methods well known by a person of ordinary skill in the art. Generally, this involves inserting a nucleic acid molecule capable of expressing a reporter molecule into an expression system to which the reporter molecule is heterologous (i.e., not normally present). The introduction of a particular foreign or native gene into a host may be facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in sense (5′→3′) orientation and correct reading frame. The vector contains the necessary elements for the transcription and translation of the inserted protein coding sequences.

Alternatively, the reporter molecule may also be integrated into the bacterial genome instead of maintained on a nucleic acid vector.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express a reporter molecule a cell. Primarily, the vector system must be compatible with the host cell used. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used.

Depending on the vector system and host utilized, any number of suitable transcription and/or translation elements, including constitutive, inducible, and repressible promoters, as well as minimal 5′ promoter elements may be used.

The protein-encoding nucleic acid, a promoter molecule of choice, a suitable 3′ regulatory region and, if desired, polyadenylation signals and/or a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a protein is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a nucleic acid construct.

Once the isolated nucleic acid molecule encoding the protein or reporter molecule has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The DNA sequences are incorporated into the host cell using standard cloning procedures known in the art, as described by Sambrook et al., Molecular Cloning: A Laboratory Manual, Second Edition, Cold Springs Laboratory, Cold Springs Harbor, New York (1989), which is hereby incorporated by reference in its entirety. Suitable hosts include, but are not limited to, bacteria, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to ampicillin, gentamycin, G418, hygromycin, puromycin, streptomycin, spectinomycin, tetracycline, chloramphenicol, and the like. Another reason antibiotic-resistant agar or media is used is to help with persistence, or the ability of bacteria to maintain plastmids over time, even under adverse conditions that would normally cause plamid loss. In terms of consistent bacterial growth, persistence is important because it allows bacteria to maintain access to the genes encoded in the plasmid. By utilizing an antibiotic-resistance agar, consistent growth of the plasmids can be achieved, leading to minimal loss of the plasmid over time. It is also important that the origin of replication, the part of the plasmid that controls plasmid DNA generation, has a high throughout, generally about 10-60 copies of plasmid per bacteria.

Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

Exemplary fluorescent polypeptides that function as reporter molecules for purposes of the methods described herein include, but are not limited to, Green Fluorescent Protein (GFP), Enhanced Green Fluorescent Protein (eGFP), Blue Fluorescent Protein (BFP), Yellow Fluorescent Protein (YFP), Cyan Fluorescent Protein (CFP), and Red Fluorescent Protein (RFP). One of skill in the art will understand that any other suitable fluorescent polypeptide, or combination of fluorescent polypeptides, can be used. In some embodiments, the reporter molecule is GFP. An exemplary nucleotide sequence for GFP is included in the vector nucleotide sequence of GenBank Accession No: EU593761, which is hereby incorporated by reference in its entirety. Specifically, the GFP sequence is the reverse complement of nucleotides 457 . . . 1176 of GenBank Accession No. EU593761, and is identified as SEQ ID NO:49 as follows:

ATGGCTAGCA AAGGAGAAGA ACTGGTACCT GGAGTTGTCC
CAATTCTTGT TGAATTAGAT GGTGATGTTA ATGGGCACAA
ATTTTCTGTC AGTGGAGAGG GTGAAGGTGA TGCTACATAC
GGAAAGCTTA CCCTTAAATT TATTTGCACT ACTGGAAAAC
TACCTGTTCC ATGGCCAACA CTTGTCACTA CTTTCTCTTA
TGGTGTTCAA TGCTTTTCCC GTTATCCGGA TCATATGAAA
CGGCATGACT TTTTCAAGAG TGCCATGCCC GAAGGTTATG
TACAGGAACG CACTATATCT TTCAAAGATG ACGGGAACTA
CAAGACGCGT GCTGAAGTCA AGTTTGAAGG TGATACCCTT
GTTAATCGTA TCGAGTTAAA AGGTATTGAT TTTAAAGAAG
ATGGAAACAT TCTCGGACAC AAACTCGAGT ACAACTATAA
CTCACACAAT GTATACATCA CGGCAGACAA ACAAAAGAAT
GGAATCAAAG CTAACTTCAA AATTCGCCAC AACATTGAAG
ATGGATCCGT TCAACTAGCA GACCATTATC AACAAAATAC
TCCAATTGGC GATGGCCCTG TCCTTTTACC AGACAACCAT
TACCTGTCGA CACAATCTGC CCTTTCGAAA GATCCCAACG
AAAAGCGTGA CCACATGGTC CTTCTTGAGT TTGTAACTGC
TGCTGGGATT ACACATGGCA TGGATGAGCT CTACAAATAA

In some embodiments, screenable markers may also be used in control bacterial strains as reporter molecules. Exemplary screenable markers include a β-glucuronidase or uidA gene (GUS) or PAD AC, a chromogenic cephalosporin that encode an enzyme for which various chromogenic substrates are known, a β-lactamase gene, a luciferase gene; ax IE gene that encodes a catechol dioxygenase that can convert chromogenic catechols, an amylase gene, and a tyrosinase gene that encodes an enzyme capable of oxidizing tyrosine to DOPA and dopaquinone, which in turn condenses to melanin.

In some embodiments, a control sample comprises a control bacterial strain engineered with a reporter molecule. The control bacterial strain engineered with a reporter molecule distinguishes a control strain from actual food contaminants or naturally occurring microorganisms and allows immediate differentiation of a control bacterial strain from detection of the target bacterial species. In some embodiments, the control bacterial strain is useful for distinguishing false positive results (i.e., cross-contamination that may have occurred during the analysis). The purpose of a quality control strain is to verify a labatory's process worked properly from the beginning to end, which in turn confirms the results of samples that were tested alongside the control strain. However, the quality control strain should be the only sample that amplifies for the reporter molecule, i.e., the GFP PCR target. For example, if a target bacterial strain is detected in a sample, the results from the control bacterial strain PCR probe specific to the GFP target can help determine if it is a false positive or not. A false positive result can occur due to cross-contamination of a test sample with neighboring samples, reagents, or lab surfaces. If no amplification of the GFP target from the control engineered bacterial strain is detected in the test sample, then the positive result for the target bacterial strain is likely to be a true positive result.

A control bacterial strain engineered with a fluorescent polypeptide reporter molecule can also produce visible fluorescent bacterial colonies on selective agar plates under UV light after isolation from an enrichment media after 24-72 hours of incubation time. Detection of reporter molecule fluorescence can be used to detect cross-contamination of test samples, since test samples should not have any visible fluorescent colonies when grown on selective agar plates. Only control samples should have fluorescent colonies when grown on selective agar plates.

In some embodiments, the control bacterial strain is a Salmonella strain engineered with Green Fluorescent Protein (GFP). That is, a gene comprising the GFP reporter molecule has been introduced into a Salmonella strain such that the Salmonella engineered strain expresses GFP and can be detected using a primer probe set specific to GFP or by detecting fluorescence of GFP in bacterial colonies using UV light. In some embodiments, the control bacterial strain is a Listeria strain engineered with Green Fluorescent Protein (GFP). In some embodiments, the control bacterial strain is a Listeria monocytogenes, Listeria grayi, Listeria ivanovii, Listeria innocua, Listeria welshmeri, Listeria seeligeri, Listeria marthii, Listeria aquatica, or Listeria riparia strain engineered with Green Fluorescent Protein (GFP). In some embodiments, the control bacterial strain is an E. coli strain engineered with Green Fluorescent Protein (GFP). In some embodiments, the control bacterial strain is an E. coli O157:H7 strain engineered with Green Fluorescent Protein (GFP). In some embodiments, the control bacterial strain is an E. coli O26, O45, O103, O111, O121, or O145 strain engineered with Green Fluorescent Protein (GFP).

In some embodiments, the test sample is from a food. In some embodiments, the food is selected from a fresh food product, such as an uncooked, raw fruit, vegetable, nut, or fungus; a processed food product, such as packaged food, frozen food, canned food, bottled food, or baked food (e.g., a meal or a bakery product); a pre-cooked, cooked, or raw meat product; a dairy product; a seafood product; a plant product; a pet food product; a chocolate product; a beverage product; or any combination thereof. In some embodiments, the food is selected from raw meat, cooked meat, rinse from a carcass, processed meat, frankfurters, sausages, jerky, shrimp, fish, egg, cheese, ice cream, butter, milk, yoghurt, vegetables, vegetable juice, pet food, nut butter, oil, leafy greens, beans, salad, fruit, fruit juice, chocolate, or any combination thereof.

In some embodiments, the test sample is an environmental sample. Exemplary environmental samples include, without limitation, surface samples, air samples, water samples, soil samples, or any other sample collected from the environment for which pathogen testing is needed, or any combination thereof. In some embodiments, the environmental sample is collected from a surface of stainless steel, plastic, rubber, ceramic tile, sealed concrete, unsealed concrete, polyolefin sample sheets, or any combination thereof.

In some embodiments the test sample is from a plant. In some embodiments, the plant is a hemp plant.

In some embodiments, the test sample is collected using a swab, a sponge, a sampling sheet, a gauze, a cotton applicator, a foam applicator, or a fabric applicator.

Selective Enrichment of Bacteria from Samples

In carrying out the methods of the present disclosure, the test sample and the control sample may be incubated, separately, prior to lysing, in an enrichment and/or selective media to enrich a target bacterial species in the test sample (if present) and the bacterial species in the control sample. As used herein, “selective media” refers to the use of a media that preferentially supports the growth of particular organisms of interest over other organisms. For example, the selective agents present in Enterohemorrhagic E. coli Recovery and Enrichment Broth (EREB) have been optimized to efficiently inhibit competing normal bacterial flora without affecting the growth of Salmonella or Enterohemorrhagic E. coli species. The selective agents present in Listeria Recovery and Enrichment Broth (LREB) have been optimized to efficiently inhibit competing normal bacterial flora without affecting the growth of Listeria species. “Enrichment media” refers to media that supports growth of the target bacterial species, and may not inhibit the growth of other bacterial flora.

In some embodiments, samples are enriched prior to or instead of use of selective media. In some embodiments, enrichment media comprises Buffered Peptone Water (BPW).

In some embodiments, the selective media comprises Enterohemorrhagic E. coli Recovery and Enrichment Broth (EREB).

In some embodiments, the selective media comprises Listeria Recovery and Enrichment Broth (LREB).

In some embodiments, the enrichment media and/or selective media is pre-warmed prior to addition of the test sample and/or the control sample. In some embodiments, the selective media is pre-warmed to about 25° C., 26° C., 27° C., 28° C. 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C., or any temperature or temperature range between about 25° C.-50° C. In some embodiments, the enrichment media and/or selective media is pre-warmed to about 30° C. In some embodiments, the enrichment media and/or selective media is pre-warmed to about 35° C. In some embodiments, the enrichment media and/or selective media is pre-warmed to about 37° C. In some embodiments, the enrichment media and/or selective media is pre-warmed to about 42° C. In some embodiments, the enrichment media and/or selective media is pre-warmed to about 45° C.

In some embodiments, the test sample and/or the control sample is incubated at a temperature of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C., or any temperature or temperature range between about 25° C.-50° C. In some embodiments, the test sample or the control sample is incubated at a temperature of about 30° C. In some embodiments, the test sample or the control sample is incubated at a temperature of about 35° C. In some embodiments, the test sample or the control sample is incubated at a temperature of about 37° C. In some embodiments, the test sample or the control sample is incubated at a temperature of about 42° C. In some embodiments, incubating is carried out at a temperature of about 30-48° C.

In some embodiments, the sample size is about 10 g, 15 g, 20 g, 25 g, 30 g, 35 g, 40 g, 45 g, 50 g, 55 g, 60 g, 65 g, 75 g, 100 g, 125 g, 150 g, 175 g, 200 g, 225 g, 250 g, 275 g, 300 g, 325 g, 350 g, 375 g, or 400 g, or any amount or size range between about 10 g-400 g. In some embodiments, the sample size is about 10 g. In some embodiments, the sample size is about 25 g. In some embodiments, the sample size is about 100 g. In some embodiments, the sample size is about 125 g. In some embodiments, the sample size is about 200 g. In some embodiments, the sample size is about 375 g. In some embodiments, the sample size is about 10 ml, 15 ml, 20 ml, 25 ml, 30 ml, 35 ml, 40 ml, 45 ml, 50 ml, 55 ml, 60 ml, 65 ml, 75 ml, 100 ml, 125 ml, 150 ml, 175 ml, 200 ml, 225 ml, 250 ml, 27 5 ml, 300 ml, 325 ml, 350 ml, 375 ml, or 400 ml, or any amount or size range between 10 ml-400 ml. In some embodiments, the sample size is about 30 ml. In some embodiments, the sample is a swab, a sample sheet, or a sponge, or is taken or obtained with a swab, a sample sheet, or a sponge. Exemplary volumes used for various sample types are provided in Tables 2, 3, 13, 20, and 21.

In some embodiments, the sample is homogenized. Homogenization of the sample is used to distribute the sample uniformly in the selective media. In some embodiments, the sample is hand mixed.

In some embodiments, the sample is incubated in about 1 ml, 2 ml, 3 ml, 4 ml, 5 ml, 6 ml, 7 ml, 8 ml, 9 ml, 10 ml, 15 ml, 20 ml, 25 ml, 30 ml, 50 ml, 75 ml, 100 ml, 125 ml, 150 ml, 175 ml, 200 ml, 225 ml, 250 ml, 300 ml, 350 ml, 400 ml, 450 ml, 500 ml, 600 ml, 700 ml, 800 ml, 900 ml, 1 L, 1.5 L, 2 L, or any specific amount or range between 1 ml and 2 L, enrichment media and/or selective media. In some embodiments, the sample is incubated in about 9 ml enrichment media and/or selective media. In some embodiments, the sample is incubated in about 30 ml enrichment media and/or selective media. In some embodiments, the sample is incubated in about 50 ml enrichment media and/or selective media. In some embodiments, the sample is incubated in about 90 ml enrichment media and/or selective media. In some embodiments, the sample is incubated in about 200 ml enrichment media and/or selective media. In some embodiments, the sample is incubated in about 225 ml enrichment media and/or selective media. In some embodiments, the sample is incubated in about 400 ml enrichment media and/or selective media. In some embodiments, the sample is incubated in about 450 ml enrichment media and/or selective media. In some embodiments, the sample is incubated in about 1 L enrichment media and/or selective media. In some embodiments, the sample is incubated in about 2 L enrichment media and/or selective media. Exemplary volumes of enrichment media and/or selective media for various sample types are provided in Examples 1-5.

In some embodiments, samples are incubated in enrichment media and/or selective media for 10-18 hours, 12-18 hours, 16-20 hours, 24 hours, 30-36 hours, or 12-25 hours, or for any specific amount of time or range of time between 10 and 36 hours. In some embodiments, incubating is carried out for about 12-25 hours. In some embodiments, samples are incubated in enrichment media and/or selective media for about 2, 4, 6, 8, 10, 12, 14, 16, 18, 20, 22, 24, 26, 28, 30, 32, 34, 36, 38, 40, 42, or 48 hours, or for any specific amount of time or range of time between 2 and 48 hours. Non-limiting examples of incubation times for various sample types are provided in Examples 1-5.

In some embodiments, the method further comprises pre-treating the test sample and/or the control sample to remove nucleic acids not associated with intact cells, thereby generating a pre-treated test sample and/or pre-treated control sample before lysing. A pre-treatment step is used in PCR to prepare the sample for amplification. This step can include various treatments such as lysis to release DNA from cells, removal of inhibitors, extraction of DNA from complex matrices, and purification of DNA. The purpose of a pre-treatment step is to obtain high-quality DNA for efficient and accurate PCR amplification. In some embodiments, Tris-EDTA (TE) buffer and Triton X-100 are used as lysing agents. In some embodiments, a 5× concentration of TE buffer, 50% trehalose solution, 40% polyethylene glycol (PEG) solution, and 1000 U/mL of mutanolysin solution are used as lysing agents. In some embodiments, a 5× concentration of TE buffer, 50% trehalose solution, 40% polyethylene glycol (PEG) solution, and 1 mg/mL of Proteinase K solution are used as lysing agents. In some embodiments, a secondary enrichment step in Brain Heart Infusion (BHI) broth may be performed.

In some embodiments, pre-treating is carried out with a pre-treatment mixture comprising a nuclease. According to these embodiments, lysing is carried out with a lysis mixture comprising an agent that inactivates the nuclease

In some embodiments, the test sample and/or the control sample is lysed to generate a test sample lysate and/or a control sample lysate. In some embodiments, an aliquot of the enriched test sample and/or the enriched control sample is lysed. In some embodiments, 1 μl, 2 μl, 3 μl, 4 μl, 5 μl, 6 μl, 7 μl, 8 μl, 9 μl, 10 μl, 15 μl, 20 μl, 25 μl or any specific amount or range between 1 μl and 25 μl of the enriched test sample or the enriched control sample is lysed. In some embodiments, 400 μl, 450 μl, 500 μl, 550 μl, 600 μl, 650 μl, 700 μl, 750 μl, 800 μl, 850 μl, 900 μl, 950 μl, 995 μl, 1 mL, or any specific amount or range between 400 μl and 1 mL of lysis buffer is used. In some embodiments, the lysis buffer may be added in the form of a lyophilized pellet. In some embodiments, two or more samples are pooled immediately after lysis. In some embodiments, about 5 μl each of up to 10 individual enriched sample lysates are pooled after lysis. In some embodiments, samples are heated at about 37° C., or 55° C. to activate an enzyme. In some embodiments, samples are heated in lysis buffer. In some embodiments, samples are heated to 90° C., 91° C., 92° C., 93° C., 94° C., 95° C., 96° C., 97° C., 98° C., 99° C., or 100° C., or any range thereof, in lysis buffer.

In some embodiments, a dilution step, such as diluting an aliquot of the lysate into Butterfield's Phosphate Diluent, as a non-limiting example, may be performed to help reduce PCR inhibition.

### Detecting Target Bacterial Species

PCR primer pairs with forward and reverse oligonucleotide primers as described herein are designed to delineate and amplify particular regions or fragments of a target bacterial species' genome or genomes using an amplification reaction such as PCR or Real Time-PCR. These exemplary amplification reactions comprise either two or three step cycles. Two step cycles have a high temperature denaturation step followed by a hybridization/elongation step. Three step cycles comprise a denaturation step, a hybridization step, and a separate elongation step. During the hybridization step, the first and/or second oligonucleotide primers of one or more primer sets as described herein hybridize to their respective target nucleotide sequence, and during the elongation step, the primers are extended to form primer extension products. The primer extension product of one primer is designed to serve as target nucleotide sequence for the other primer of the primer set in the amplification reaction. Thus, repetition of the reaction cycles results in exponential amplification of the target region encompassed by primers. This target region, defined on its 5′end by the one primer nucleotide sequence (i.e., the forward oligonucleotide primer sequence) and defined on its 3′ end by the complement of the second oligonucleotide primer sequence (i.e., the complement of the reverse oligonucleotide primer sequence), respectively, is referred to herein as the amplification product or amplicon. Other methods of multiplex amplification known to those of skill in the art, such as isothermal amplification can also be used.

In some embodiments, the amplification products generated in accordance with the methods described here are nucleic acid molecules that are at least 20 nucleotides in length. In some embodiments, the amplification products are 25, 30, 35, 40, 45, 50, 55, 60, 65, 70, 75, 80, 85, 90, 95, 100, any size or range of size between 20-100 nucleotides in length, or greater than 100 nucleotides in length.

Detection of the PCR product can be carried out using a fluorescent qPCR detection assay such as the TaqMan™ assay (see U.S. Pat. No. 6,270,967 to Whitcombe et al., and U.S. Pat. No. 7,601,821 to Anderson et al., which are hereby incorporated by reference in their entirety). For detection using TaqMan™ an oligonucleotide probe spanning the target region is used in conjunction with primers suitable for hybridization on the primer-specific portions of the secondary PCR products for amplification and detection. Such PCR primer sets that include a probe are referred to herein as a PCR probe set. A TaqMan™ probe contains a fluorescent reporter group on one end and a quencher molecule on the other end that are in close enough proximity to each other in the intact probe that the quencher molecule quenches fluorescence of the reporter group. During amplification, the TaqMan™ probe and upstream primer hybridize to their complementary regions of the ligation product. The 5′→3′ nuclease activity of the polymerase extends the hybridized primer and liberates the fluorescent group of the TaqMan™ probe to generate a detectable signal. Exemplary PCR probe sets used in the methods of the present disclosure are optimized to perform in a multiplex reaction. Multiple probe sets can be detected in the same multiplex reaction by using different fluorescent groups on the TaqMan™ probes.

A multiplexed PCR amplification reaction is carried out using target specific primers and probes and a PCR mix to detect specific target bacteria and/or bacterial genes. In some embodiments, real time fluorescent quantitative PCR assay are performed. In some embodiments, Taq enzyme, PCR reaction buffer, dNTP mixture, target bacterial genes, and PCR probe sets comprising specific upstream and downstream primers and fluorescent probes are used. As an increasing number of targets are amplified within a sample, selective amplification while avoiding the formation of artifacts, such as primer-dimers, increases. The exemplary PCR primers and PCR probe sets of the present disclosure have been optimized to reduce artefacts.

Table 1 shows the sequences of exemplary PCR primers and PCR probe sets useful for multiplex amplification and detection of target bacterial species. In some embodiments, the primers amplify a target. In some embodiments, the amplified target distinguishes a target bacterial species from other target bacterial species. In some embodiments, a target is amplified in multiple species from a bacterial genus. In some embodiments, the amplified target is detected by a fluorescently-labeled probe. In some embodiments, each of the amplified target and the amplified control is detected by a probe comprising a different fluorescent label.

Primers comprise a nucleotide sequence that is complementary or substantially complementary to a target nucleotide sequence. The terms “complementary” and “substantially complementary” refer to base pairing between nucleotides such as, for instance, between an oligonucleotide primer and its target nucleotide sequence. Complementary nucleotides are generally adenine and thymine, adenosine and uracil, and guanine and cytosine. Within the context of the methods disclosed herein, the oligonucleotide primers do not require complete complementarity in order to hybridize to their target nucleotide sequence. The primer sequences disclosed herein may be modified to some extent without loss of utility as specific primers. Exemplary PCR primer sets and PCR probe sets used in various embodiments are shown in Table 1. PCR primer sets for each Assay Target comprise the Forward (F) and Reverse (R) primer. PCR probe sets for each Assay Target comprise the Forward (F) primer, the Reverse (R) primer, and the Probe.

TABLE 1
Exemplary PCR Primer Sets and PCR Probe Sets

| | SEQ | | |
|---|---|---|---|
| | ID | | |
| Assay Target | NO: | ID | Sequence (5′ to 3′) |
| Salmonella 1 | 1 | F | AGCGTACTGGAAAGGGAAAG |
| | 2 | R | ATACCGCCAATAAAGTTCACAAAG |
| | 3 | Probe | CGTCACCTTTGATAAACTTCATCGCA |
| Salmonella 2 | 4 | F | AGCAAACCCAGGTGAAACTGA |
| | 5 | R | AGGCTTACCGTCAAAAGTTAACGT |
| | 6 | Probe | TCACTGGCACAGGCATTCCATCG |
| GFP | 7 | F | CATTGAAGATGGATCCGTTCAACTAGCAG |
| | 8 | R | ATCCCAGCAGCAGTTACAAACTCAAGA |
| | 9 | Probe | AGGACAGGGCCATCGCCAATTGGAGTATTT |
| Listeria spp. | 10 | F | AGGATAGGGAATCGCACGAA |
| | 11 | R | TTCGCGAGAAGCGGATTT |
| | 12 | Probe | TCTCACACTCACTGCTTGGACGC |
| Listeria | 13 | F | CATGGCACCACCAGCATCT |
| monocytogenes | 14 | R | ATCCGCGTGTTTCTTTTCGA |
| | 15 | Probe | CGCCTGCAAGTCCTAAGACGCCA |
| E. coli O157: H7 | 16 | F | TATTCCGCGATGCTTGTTTTT |
| | 17 | R | ATTATCTCACCAGCAAACTGGCGG |
| | 18 | Probe | CCGCAATCTTTCC |
| Shiga Toxin 1 (stx) | 19 | F | GTGGCATTAATACTGAATTGTCATCA |
| | 20 | R | GCGTAATCCCACGGACTCTTC |
| | 21 | Probe | TGATGAGTTTCCTTCTATGTGTCCGGCAGAT |
| Shiga Toxin 2 (stx) | 22 | F | GATGTTTATGGCGGTTTTATTTGC |
| | 23 | R | TGGAAAACTCAATTTTACCTTTAGCA |
| | 24 | Probe | TCTGTTAATGCAATGGCGGCGGATT |
| Intimin (eae) | 25 | F | CCGATTCCTCTGGTGACGA |
| | 26 | R | CCACGGTTTATCAAACTGATAACG |
| | 27 | Probe | CGTCATGGTACGGGTAA |
| E. coli O103 | 28 | F | TTGGAGCGTTAACTGGACCT |
| | 29 | R | ATATTCGCTATATCTTCTTGCGGC |
| | 30 | Probe | AGGCTTATCTGGCTGTTCTTACTACGGC |
| E. coli O26 | 31 | F | GTATCGCTGAAATTAGAAGCGC |
| | 32 | R | AGTTGAAACACCCGTAATGGC |
| | 33 | Probe | TGGTTCGGTTGGATTGTCCATAAGAGGG |
| E. coli O145 | 34 | F | AAACTGGGATTGGACGTGG |
| | 35 | R | CCCAAAACTTCTAGGCCCG |
| | 36 | Probe | TGCTAATTGCAGCCCTTGCACTACG |
| E. coli O111 | 37 | F | TGTTCCAGGTGGTAGGATTCG |
| | 38 | R | TCACGATGTTGATCATCTGGG |
| | 39 | Probe | TGAAGGCGAGGCAACACATTATATAGTGC |
| E. coli 45 | 40 | F | CGTTGTGCATGGTGGCAT |
| | 41 | R | TGGCCAAACCAACTATGAACTG |
| | 42 | Probe | ATTTTTTGCTGCAAGTGGGCTGTCCA |
| E. coli O121 | 43 | F | AGGCGCTGTTTGGTCTCTTAGA |
| | 44 | R | GAACCGAAATGATGGGTGCT |
| | 45 | Probe | CGCTATCATGGCGGGACAATGACA |
| IAC | 46 | F | CTAACCTTCGTGATGAGCAATCG |
| | 47 | R | GATCAGCTACGTGAGGTCCTAC |
| | 48 | Probe | AGCTAGTCGATGCACTCCAGTCCTCC |

In some embodiments, the test sample lysate and the control sample lysate are contacted with an assay mixture comprising one or more bacterial detection PCR primer sets, and a control detection PCR primer set, thereby generating one or more assay samples capable of amplifying a target. In some embodiments, the assay mixture comprises one or more PCR primer sets. In some embodiments, a PCR primer set comprises a forward primer and a reverse primer. In some embodiments, the assay mixture comprises one or more PCR probe sets. In some embodiments, a PCR probe set comprises a forward primer, a reverse primer, and a probe. In some embodiments, the probe comprises a fluorescent reporter group on one end and a quencher molecule on the other end. In some embodiments, each PCR probe set in a multiplex PCR reaction has a probe labeled with a different fluorescent molecule.

Non-limiting examples of fluorescent molecules include, for example, a red fluorescent squarine dye such as e.g., 2,4-Bis[1,3,3-trimethyl-2-indolinylidenemethyl]cyclobutenediylium-1,3-dioxolate; an infrared dye, e.g., 2,4Bis[3,3-dimethyl-2-(1H-benz[e]indolinylidenemethyl)]cyclobutenediylium-1,3-dioxolate; or an orange fluorescent squarine dye such as, e.g., 2,4-Bis[3,5-dimethyl-2-pyrrolyl]cyclobutenediylium-1,3-diololate. Additional non-limiting examples of fluorophores include quantum dots, Alexa Fluor® dyes, AMCA, Biosearch Blue, BODIPY® 630/650, BODIPY® 650/665, BODIPY®-FL, BODIPY®-R6 G, BODIPY®-TMR, BODIPYR-TRX, Calcein, Calcium Green™, Calcium Crimson™, Calcium Orange™, Cascade Blue®, CAL Fluor Gold 540, CAL Fluor Orange 560, CAL Fluor Red 590, CAL Fluor Red 610, CAL Fluor Red 635, CyDye™, including but not limited to Cy2™, Cy3™, Cy3.5™, and Cy5™, Cy5.5, a DNA intercalating dye, DiD DilC (5), FAM, 6-FAM™, Fluorescein, Fluor X™, HEX™, 6-JOE, Magnesium Green™, Magnesium Orange™, Nile Red, Oregon Green® 488, Oregon Green® 500, Oregon Green® 514, Pacific Blue™, REG, phycobilliproteins including, but not limited to, phycoerythrin, PyroninY allophycocyanin, Pulsar 650, RiboGreen™, Rhodamine B, Rhodamine Green™, Rhodamine Red™, Rhodamine 110, Rhodamine 123, Rhodamine Phalloidin, ROX™, R-phycocyanin, TAMRA™, TET™, Tetramethylrhodamine, Texas Red®, Thiadicarbocyanine, TO-PRO™-1, TO-PRO™-3,TOTO1, TOTO3, TRITC, YOPRO™-1, YOYO™-1, YOPRO™-3, YOYO™-3, Quasar 570, Quasar 670, and Quasar 705.

In some embodiments, molecules useful as quenchers include, but are not limited to, tetramethylrhodamine (TAMRA), DABCYL (DABSYL, DABMI or methyl red) anthroquinone, nitrothiazole, nitroimidazole, malachite green, Black Hole Quenchers®, e.g., BHQI (Biosearch Technologies), Iowa Black® or ZEN quenchers (from Integrated DNA Technologies, Inc.) and TIDE Quenchers (e.g. TID Quencher 2 (TQ2) and TIDE Quencher 3 (TQ3)) (from AAT Bioquest). In some embodiments, the probes used in the methods described herein comprise an internal quencher. In some embodiments, the probes used in the methods described herein comprise 3′ quencher. In some embodiments, the probes used in the methods described herein comprise two quencher molecules, an internal quencher and a 3′ quencher.

In some embodiments, the target bacterial species is a Salmonella species. In some embodiments, the assay mixture comprises (i) one or more PCR primer sets capable of detecting Salmonella and (ii) one or more PCR primer sets capable of detecting a reporter molecule. In some embodiments, the one or more PCR primer sets detect Salmonella species. In some embodiments, the one or more PCR primer sets comprise: Salmonella 1 (SEQ ID NOs: 1-2) and Salmonella 2 (SEQ ID NOs:4-5). In some embodiments, the one or more PCR primer sets detect Salmonella species and a control bacterial strain engineered with a reporter molecule. In some embodiments, the one or more PCR primer sets comprise: Salmonella 1 (SEQ ID NOs:1-2), Salmonella 2 (SEQ ID NOs:4-5), and Green Fluorescent Protein (GFP) (SEQ ID NOs:7-8). In some embodiments, the assay mixture further comprises an internal amplification control (IAC) PCR primer set as an assurance that PCR amplification has taken place. In some embodiments, the internal amplification control PCR primer set comprises (SEQ ID NOs:46-47).

In some embodiments, the assay mixture comprises (i) one or more PCR probe sets capable of detecting Salmonella and (ii) one or more PCR probe sets capable of detecting a reporter molecule. In some embodiments, the one or more PCR probe sets detect Salmonella species. In some embodiments, the one or more PCR probe sets comprise: Salmonella 1 (SEQ ID NOs: 1-3) and Salmonella 2 (SEQ ID NOs:4-6). In some embodiments, the one or more PCR probe sets detect Salmonella species and a control bacterial strain engineered with a reporter molecule. In some embodiments, the one or more PCR probe sets comprise: Salmonella 1 (SEQ ID NOs:1-3), Salmonella 2 (SEQ ID NOs:4-6), and Green Fluorescent Protein (GFP) (SEQ ID NOs:7-9). In some embodiments, the assay mixture further comprises an internal amplification control (IAC) PCR probe set as an assurance that PCR amplification has taken place. In some embodiments, the internal amplification control PCR probe set comprises (SEQ ID NOs:46-48).

In some embodiments, the target bacterial species is a Listeria species. In some embodiments, the assay mixture comprises (i) one or more PCR primer sets capable of detecting Listeria and (ii) one or more PCR primer sets capable of detecting a reporter molecule. In some embodiments, the one or more PCR primer sets detect Listeria species. In some embodiments, the one or more PCR primer sets comprise: Listeria spp. (SEQ ID NOs: 10-11) and Listeria monocytogenes (SEQ ID NOs:13-14). In some embodiments, the one or more PCR primer sets detect Listeria species and a control bacterial strain engineered with a reporter molecule. In some embodiments, the one or more PCR primer sets comprise: Listeria spp. (SEQ ID NOs: 10-11), Listeria monocytogenes (SEQ ID NOs:13-14) and Green Fluorescent Protein (GFP) (SEQ ID NOs:7-8). In some embodiments, the assay mixture further comprises an internal amplification control (IAC) PCR primer set as an assurance that PCR amplification has taken place. In some embodiments, the internal amplification control PCR primer set comprises (SEQ ID NOs:46-47).

In some embodiments, the assay mixture comprises (i) one or more PCR probe sets capable of detecting Listeria and (ii) one or more PCR probe sets capable of detecting a reporter molecule. In some embodiments, the one or more PCR probe sets detect Listeria species. In some embodiments, the one or more PCR probe sets comprise: Listeria spp. (SEQ ID NOs: 10-12) and Listeria monocytogenes (SEQ ID NOs: 13-15). In some embodiments, the one or more PCR probe sets detect Listeria species and a control bacterial strain engineered with a reporter molecule. In some embodiments, the one or more PCR probe sets comprise: Listeria spp. (SEQ ID NOs: 10-12), Listeria monocytogenes (SEQ ID NOs: 13-15) and Green Fluorescent Protein (GFP) (SEQ ID NOs: 7-9). In some embodiments, the assay mixture further comprises an internal amplification control (IAC) PCR probe set as an assurance that PCR amplification has taken place. In some embodiments, the internal amplification control PCR probe set comprises (SEQ ID NOs:46-48).

In some embodiments, the target bacterial species is an E. coli species. In some embodiments, the E. coli species is a Shiga toxin producing E. coli (STEC). In some embodiments, the assay mixture comprises (i) one or more PCR primer sets capable of detecting E. coli and (ii) one or more PCR primer sets capable of detecting a reporter molecule. In some embodiments, the one or more PCR primer sets detect E. coli. In some embodiments, the one or more PCR primer sets detect STEC. In some embodiments, the one or more PCR primer sets comprise: E. coli O157:H7 (SEQ ID NOs: 16-17). In some embodiments, the one or more PCR primer sets detect Escherichia species and a control bacterial strain engineered with a reporter molecule. In some embodiments, the one or more PCR primer sets comprise: E. coli O157:H7 (SEQ ID NOs: 16-17) and Green Fluorescent Protein (GFP) (SEQ ID NOs:7-8). In some embodiments, the one or more PCR primer sets comprise: Shiga toxin (stx) 1 (SEQ ID NOs: 19-20) and Shiga toxin (stx) 2 (SEQ ID NOs:22-23). In some embodiments, the one or more PCR probe sets comprise: Shiga toxin (stx) 1 (SEQ ID NOs:19-20) and the Intimin gene (eae) (SEQ ID NOs:25-26). In some embodiments, the one or more PCR primer sets comprise: Shiga toxin (stx) 2 (SEQ ID NOs:22-23) and the Intimin gene (eae) (SEQ ID NOs:25-26). In some embodiments, the one or more PCR primer sets comprise: E. coli 0103 (SEQ ID NOs:28-29) and E. coli 026 (SEQ ID NOs:31-32). In some embodiments, the one or more PCR primer sets comprise: E. coli 0145 (SEQ ID NOs:34-35) and E. coli 0111 (SEQ ID NOs:37-38). In some embodiments, the one or more PCR primer sets comprise: E. coli 045 (SEQ ID NOs:40-41) and E. coli 0121 (SEQ ID NOs:43-44). In some embodiments, the assay mixture further comprises an internal amplification control (IAC) PCR primer set as an assurance that PCR amplification has taken place. In some embodiments, the internal amplification control PCR primer set comprises (SEQ ID NOs:46-47).

In some embodiments, the E. coli species is a Shiga toxin producing F. coli (STEC). In some embodiments, the assay mixture comprises (i) one or more PCR probe sets capable of detecting E. coli and (ii) one or more PCR probe sets capable of detecting a reporter molecule. In some embodiments, the one or more PCR probe sets detect E. coli. In some embodiments, the one or more PCR probe sets detect STEC. In some embodiments, the one or more PCR probe sets comprise: E. coli O157:H7 (SEQ ID NOs: 16-18). In some embodiments, the one or more PCR probe sets detect Escherichia species and a control bacterial strain engineered with a reporter molecule. In some embodiments, the one or more PCR probe sets comprise: E. coli O157:H7 (SEQ ID NOs: 16-18) and Green Fluorescent Protein (GFP) (SEQ ID NOs:7-9). In some embodiments, the one or more PCR probe sets comprise: Shiga toxin (stx) 1 (SEQ ID NOs: 19-21) and Shiga toxin (stx) 2 (SEQ ID NOs:22-24). In some embodiments, the one or more PCR probe sets comprise: Shiga toxin (stx) 1 (SEQ ID NOs:19-21) and the Intimin gene (eae) (SEQ ID NOs:25-27). In some embodiments, the one or more PCR probe sets comprise: Shiga toxin (stx) 2 (SEQ ID NOs:22-24) and the Intimin gene (eae) (SEQ ID NOs:25-27). In some embodiments, the one or more PCR probe sets comprise: E. coli 0103 (SEQ ID NOs:28-30) and E. coli 026 (SEQ ID NOs:31-33). In some embodiments, the one or more PCR probe sets comprise: E. coli 0145 (SEQ ID NOs:34-36) and E. coli 0111 (SEQ ID NOs:37-39). In some embodiments, the one or more PCR probe sets comprise: E. coli 045 (SEQ ID NOs:40-42) and E. coli 0121 (SEQ ID NOs:43-45). In some embodiments, the assay mixture further comprises an internal amplification control (IAC) PCR probe set as an assurance that PCR amplification has taken place. In some embodiments, the internal amplification control PCR probe set comprises (SEQ ID NOs:46-48).

In some embodiments, the one or more PCR primer sets comprise one or more of E. coli O157:H7 (SEQ ID NOs:16-17), Shiga toxin gene(stx) 1 (SEQ ID NOs: 19-20), Shiga toxin gene(stx) 2 (SEQ ID NOs:22-23), Intimin gene (eae) (SEQ ID NOs: 25-26), E. coli 0103 (SEQ ID NOs:28-39), E. coli 026 (SEQ ID NOs:31-32), E. coli 0145 (SEQ ID NOs:34-35), E. coli 0111 (SEQ ID NOs:37-38), E. coli 045 (SEQ ID NOs:40-41), E. coli 0121 (SEQ ID NOs:43-44), Green Fluorescent Protein (GFP) (SEQ ID NOs:7-8), IAC (SEQ ID NOs: 46-47), or any combination thereof.

In some embodiments, the one or more PCR probe sets comprise one or more of E. coli O157:H7 (SEQ ID NOs:16-18), Shiga toxin gene(stx) 1 (SEQ ID NOs: 19-21), Shiga toxin gene(stx) 2 (SEQ ID NOs:22-24), Intimin gene (eae) (SEQ ID NOs: 25-27), E. coli 0103 (SEQ ID NOs:28-30), E. coli 026 (SEQ ID NOs:31-33), E. coli 0145 (SEQ ID NOs:34-36), E. coli 0111 (SEQ ID NOs:37-39), E. coli 045 (SEQ ID NOs:40-42), E. coli 0121 (SEQ ID NOs:43-45), Green Fluorescent Protein (GFP) (SEQ ID NOs:7-9), IAC (SEQ ID NOs: 46-48), or any combination thereof. Other PCR primers and/or probes can also be used for detecting bacterial species.

In some embodiments, the presence or absence of an amplified target PCR product and/or an amplified control PCR product in each of the one or more assay samples is detected, thereby determining the presence or absence of the target bacterial species and/or the control bacterial strain in the samples simultaneously. In some embodiments, detecting presence or absence of an amplified target or a control in the methods described herein is carried out with fluorescence imaging. In some embodiments, detecting is carried out using real time PCR, also known as qPCR. DNA quantification by qPCR frequently relies on plotting fluorescence against the number of cycles on a logarithmic scale. A threshold for detection of DNA-based fluorescence is set slightly above background. The number of cycles at which the fluorescence exceeds the threshold is called the threshold cycle (Ct) or, according to the MIQE guidelines, quantification cycle (Cq) (Bustin et al., “The MIQE guidelines: Minimum Information for Publication of Quantitative Real-Time PCR Experiments,” Clinical Chemistry 55:611-622 (2009), which is hereby incorporated by reference in its entirety). During the exponential amplification phase, the sequence of the DNA target doubles every cycle. For example, a DNA sample whose Cq precedes that of another sample by 3 cycles contained 2³=8 times more template. However, the efficiency of amplification is often variable among primers and templates. Therefore, the efficiency of a primer-template combination can be assessed in a titration experiment with serial dilutions of DNA template to create a standard curve of the change in Cq with each dilution. The slope of the linear regression can then be used to determine the efficiency of amplification, which is 100% if a dilution of 1:2 results in a Cq difference of 1.

In qPCR, a template (the DNA to be amplified) can be prepared and the PCR probe set(s) can be added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a cycler, and its geometric increase corresponding to exponential increase of the product is used to determine the quantification cycle (Cq) in each reaction. The reaction conditions, including annealing temperature, number of cycles, length and temperature of each cycle, can be determined for each group of primers. Exemplary reaction conditions are found in Example 1.

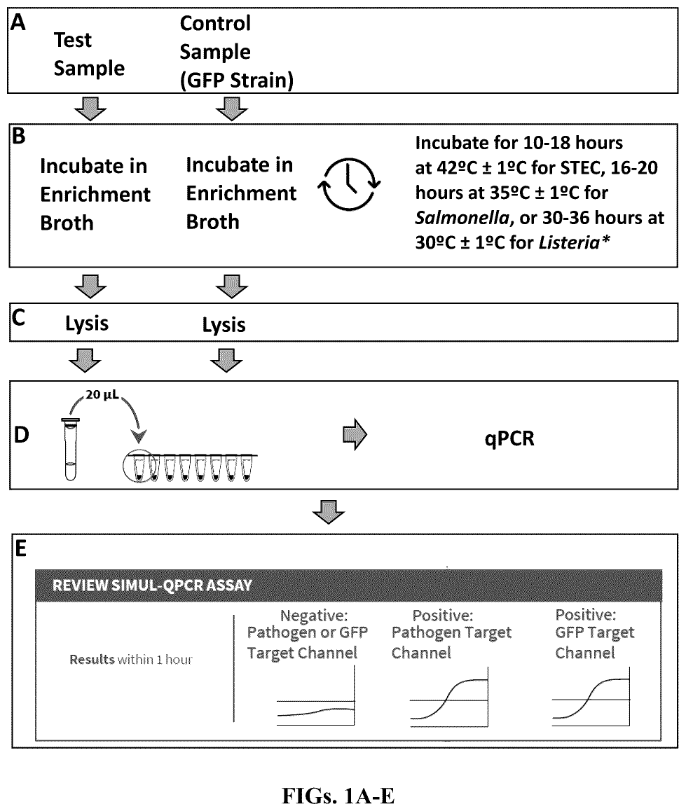

In some embodiments, detecting the presence or absence of an amplified target PCR product and/or an amplified control PCR product is determined by detecting a quantification cycle “Cq” value, during the real time PCR reaction. A qPCR reaction that has a positive Cq value and has a typical sigmoidal curve is considered positive for the target or control (FIG. 1E). A sample that does not have a sigmoidal curve that passes the Cq value, or has a non-sigmoidal curve is not considered positive for the target or control (FIG. 1E). In some embodiments, determining if cross-contamination of the test sample occurred is by detecting if the amplified control PCR product is present in the test sample.

In some embodiments, detecting the presence or absence of an amplified target PCR product and/or an amplified control PCR product comprises determining if a Cq value is present or absent. As described above, the Cq value is a threshold number of cycles at which the fluorescence exceeds a certain value in a PCR reaction having a typical sigmoidal curve (see FIG. 1E and Examples 1-5). In some embodiments, the Cq value of the amplified control PCR product occurs at about the same time or prior to the Cq value of the amplified test sample, if target bacteria are present in the test sample. The methods and kits of the present disclosure were developed such that the Cq of the GFP (control) target occurs at about the same time or earlier than the pathogen of interest (target bacteria). This was to prevent potential issues such as the following. If the amplification of the target for the pathogen of interest was detected at a much lower Cq value than the GFP target, it was possible the target for the GFP would not be detectable, which would prevent detection of cross-contamination. If the GFP target amplified a few cycles after the target pathogen, the amplification could occur too late and the GFP target would not be detected in time.

In some embodiments, the Cq values for the amplified control sample and the amplified test sample are within 0, 1, 2, 3, or 4 PCR cycles or any or any number or range therein, if target bacteria are present in the test sample.

Another aspect of the present disclosure relates to a kit comprising one or more PCR primer sets capable of detecting Salmonella, Listeria, or E. coli species and one or more PCR primer sets capable of detecting a control bacterial strain engineered with a reporter molecule.

This aspect of the present application can be carried out with any of the embodiments disclosed herein.

In some embodiments, the one or more PCR primer sets are selected from the group consisting of: (i) Salmonella 1 (SEQ ID NOs: 1-2), Salmonella 2 (SEQ ID NOs:4-5), and Green Fluorescent Protein (GFP) (SEQ ID NOs:7-8); (ii) Listera spp. (SEQ ID NOs:10-11), Listeria monocytogenes (SEQ ID NOs: 13-14), and Green Fluorescent Protein (GFP) (SEQ ID NOs:7-8); (iii) two or more of E. coli O157:H7 (SEQ ID NOs: 16-17), Shiga toxin 1 (SEQ ID NOs: 19-20), Shiga toxin 2 (SEQ ID NOs:22-23), Intimin (SEQ ID NOs:25-26), E. coli 0103 (SEQ ID NOs:28-29), E. coli 026 (SEQ ID NOs:31-32), E. coli 0145 (SEQ ID NOs:34-35), E. coli 0111 (SEQ ID NOs:37-38), E. coli 045 (SEQ ID NOs:40-41), E. coli 0121 (SEQ ID NOs:43-44), and Green Fluorescent Protein (GFP) (SEQ ID NOs:7-8); and (iv) any combination thereof. In some embodiments, the one or more PCR primer set comprises an internal amplification control (IAC) (SEQ ID NOs:46-47).

In some embodiments, the one or more PCR probe sets are selected from the group consisting of: (i) Salmonella 1 (SEQ ID NOs:1-3), Salmonella 2 (SEQ ID NOs:4-6), and Green Fluorescent Protein (GFP) (SEQ ID NOs:7-9); (ii) Listera spp. (SEQ ID NOs: 10-12), Listeria monocytogenes (SEQ ID NOs: 13-15), and Green Fluorescent Protein (GFP) (SEQ ID NOs:7-9); (iii) two or more of E. coli O157:H7 (SEQ ID NOs: 16-18), Shiga toxin 1 (SEQ ID NOs: 19-21), Shiga toxin 2 (SEQ ID NOs:22-24), Intimin (SEQ ID NOs:25-27), E. coli O103 (SEQ ID NOs:28-30), E. coli O26 (SEQ ID NOs:31-33), E. coli O145 (SEQ ID NOs:34-36), E. coli O111 (SEQ ID NOs:37-39), E. coli O45 (SEQ ID NOs:40-42), E. coli O121 (SEQ ID NOs:43-45), and Green Fluorescent Protein (GFP) (SEQ ID NOs:7-9); and (iv) any combination thereof. In some embodiments, the one or more PCR probe set comprises an internal amplification control (IAC) (SEQ ID NOs:46-48).

Such kits will contain a reagent combination including the particular elements required to conduct an assay according to the methods disclosed herein. The reagent system is presented in a commercially packaged form, as a composition or admixture where the compatibility of the reagents will allow, in a test device configuration, or more typically as a test kit, (i.e., a packaged combination of one or more containers, devices, or the like holding the necessary reagents, and preferably including written instructions for the performance of assays). The kit may be adapted for any configuration of an assay and may include compositions for performing any of the various assay formats described herein.

In some embodiments, the kit contains PCR reagents. In some embodiments, the kit contains instructions for selective enrichment of target bacterial species, selective media use, time and temperature of incubation, use of control bacterial species, PCR programs, data interpretation, and storage. The kits can include a plurality of containers reflecting the number of assays to be performed.

### EXAMPLES

### Example 1—Salmonella with GFP Assay

A protocol was developed as a multifaceted approach to the detection of Salmonella species in a variety of food products and environmental samples. The workflow of this protocol is illustrated in FIGS. 1A-E. Specifically formulated media were utilized for enriching samples followed by cultural (detection plate) and rapid (qPCR) detection procedures.

The SIMUL-qPCR System is described below, as a quick and reliable method for detecting Salmonella in food and environmental samples. All SIMUL-qPCR System assays were designed to have the same instrument run time allowing simultaneous identification of all SIMUL-qPCR System assay targets (described in Examples 1-4) in less than an hour. In addition, each assay utilizes the power of multiplexing several targets during the same run.

The SIMUL-qPCR Salmonella with GFP Assay incorporates a real-time PCR approach to identifying Salmonella and brings an added layer of assurance with the addition of the Green Fluorescent Protein (GFP) positive control.

Salmonella does not naturally contain GFP. By utilizing a Salmonella quality control strain engineered with GFP, it can be determined if any potential positives were caused by cross-contamination. Previously, potential positives would need to be streaked for cultural confirmation and then the colonies would need to be examined under a UV light with the appropriate wavelength to detect GFP fluorescence in the bacterial colonies, adding 1-3 days to the process of determining whether any cross-contamination had occurred. With the assay described herein, contamination can be determined by the end of the PCR run. The test sample and control sample (strain engineered with GFP) are shown in FIG. 1A.

The Simultaneous Multiplex Real Time PCR (SIMUL-qPCR) Salmonella with GFP Assay was validated for a variety of foods and environmental surfaces. USDA FSIS and FDA BAM methods were used for method comparison testing. See U.S. Department of Agriculture Food Safety and Inspection Service, “Isolation and Identification of Salmonella from Meat, Poultry, Pasteurized Egg, and Catfish Products and Carcass and Environmental Sponges”, Microbiology Laboratory Manual 4.09 (2017); Andrews et al., “Bacteriological Analytical Manual (BAM) Chapter 5: Salmonella.” U.S. Food and Drug Administration Bacteriological Analytical Manual 110 (2018), which are hereby incorporated by reference in their entirety. The SIMUL-qPCR Salmonella with GFP Assay was found to be equivalent to the reference methods. The limit of detection for the SIMUL-qPCR assay was determined to be 10,000 CFU/mL of enriched sample.

### Selective Enrichment Media

Test samples and the control sample (strain engineered with GFP) were enriched by incubation in enrichment broth as shown in FIG. 1B. Buffered Peptone Water (BPW) contains necessary nutritional components for the growth of Salmonella. When detecting both Salmonella and E. coli O157:H7 in the same enrichment (see Example 4), Enterohemorrhagic E. coli Recovery and Enrichment Broth (EREB) may also be used. EREB combines those nutritional components incorporated in BPW with additional ingredients that are necessary to selectively improve recovery and growth of Salmonella. The selective agents present in EREB have been optimized to efficiently inhibit competing bacterial flora without affecting the growth of Salmonella species. BPW and EREB were formulated for buffering capacity to ensure growth in a variety of matrices. The enrichment step is shown in FIG. 1B. The recipe for BPW for one liter of solution is as follows: 10 grams of peptone, 5 grams of sodium chloride, 3.5 grams of disodium phosphate, and 1.5 grams of monopotassium phosphate. The formulation for EREB for one of solution is as follows: 17 grams of tryptone, 3 grams of soytone, 5 grams of sodium chloride, 4 grams of dipotassium phosphate, 2.5 grams of dextrose, 5 grams of casein acid hydrolysate, 1 gram of bile salt, and 0.25 grams of sodium pyruvate.

### Environmental Surface Sample Preparation

Sponge: When ready to test, the prepared BPW was warmed to 45±1° C. and added to each sponge sample in its sample bag. Acceptable media volumes are shown in Table 2. Enrichment incubation for Table 2 samples is 35±1° C. for 16-20 hours unless otherwise indicated. The sample was homogenized for 30 seconds in a stomacher/blender or equivalent. Hand mixing can be used as an alternative for stomaching. To hand mix, each sponge that is in the sealed bag was massaged for approximately one minute. The sample was then incubated.

TABLE 2
BPW Enrichment Media Volumes for Samples

| | SAMPLE SIZE/ | | MEDIA |
|---|---|---|---|
| MATRIX | ANALYSIS UNIT | | VOLUME |
| Environmental Swab* | /Swab | | 9 mL ± 1 mL |
| AFD MEMP Reserve Swab* | /Swab | | 9 mL ± 1 mL |
| Environmental Sponge* | /Sponge | | 90 mL ± 10 mL |
| Poultry Carcass or Parts Rinse | /30 | mL | 30 mL ± 5 mL |
| Raw Ground Poultry | /375 | g | 1 L ± 50 mL |
| RTE Cooked Poultry | /375 | g | 2 L ± 100 mL |
| Dry Pet Food** | /375 | g | 2 L ± 100 mL |
| Pasteurized Liquid Eggs | /100 | g | 900 mL ± 18 mL |
| Frankfurter/Sausage | /25 | g | 225 mL ± 15 mL |
| Chocolate*** | /25 | g | 225 mL ± 15 mL |
| Peanut Butter | /25 | g | 225 mL ± 15 mL |
| Dried Hemp Flower**** | /10 | g | 90 mL ± 10 mL |

*Validated surfaces are stainless steel, plastic, rubber, ceramic tile, and sealed concrete
**BPW for dry pet food enrichment should be pre-warmed to 35 ± 1° C.
***BPW for chocolate enrichment should be pre-warmed to 35 ± 1° C. Chocolate has a secondary enrichment step - after incubation, pipette 20 uL of sample into 1980 uL of BHI and incubate for an additional 3-4 hours.
****BPW for dried hemp flower should be pre-warmed to 37 ± 1° C., and samples should be enriched for 24 hours, not 16-20 hours.

Swab: When ready to test, the prepared BPW was warmed to 45±1° C., and 9 mL of the pre-warmed BPW was added to each swab sample in its vial. The sample was incubated. The Enrichment of Samples Table 3 shows various enrichment conditions.

### Enrichment for Food Sample

Food Sample: In general, food products were aseptically sampled or the rinse poured into a sterile bag. When ready to be tested, the prepared medium was pre-warmed (45±1° C., 37±1° C., or 35±1° C., depending on the matrix). The pre-warmed BPW or EREB was then to the sample, as appropriate for the sample type. Enrichment of Samples Table 2 and Table 3 show acceptable media volumes by sample type.

For example, trimmed raw beef (20% fat) and raw ground beef (20% fat) were prepared for testing. A 375 g portion was aseptically sampled and placed in a sterile bag. 1 L±50 mL of pre-warmed EREB was added to the sample and bag was sealed. Each sample was hand-mixed by massaging for approximately one minute to homogenize the sample. The samples were incubated at 42° C.±1° C. for 10-18 h. If the sample lysates were being pooled, the incubation time was increased to 12-18 h.

Raw ground poultry and ready to eat cooked poultry were aseptically sampled (375 g) and placed in a sterile bag. 1 L±50 mL of pre-warmed BPW was added to the sample and the bag sealed. each sample was hand mixed by massaging for approximately one minute to homogenize. The samples were incubated at 35±1° C. for 16-20 h.

Peanut butter and frankfurters/sausages were aseptically sampled (25 g) and each placed separately in a sterile bag. 225 mL±15 mL of pre-warmed BPW were added to the samples and the bags were sealed. The samples were hand mixed by massaging or stomaching for approximately one minute to homogenize. The samples were incubated at 35±1° C. for 16-20 h.

To prepare poultry carcass rinses, 30 mL of the rinse was aseptically poured into a sterile bag. 30 mL±5 mL of pre-warmed BPW was added to the sample and the bag sealed. Each sample was hand mixed by massaging for approximately one minute to homogenize. The samples were incubated at 35±1° C. for 16-20 h.

Dry pet food was aseptically sampled (375 g) and placed in a sterile bag. 2 L±100 mL of BPW (pre-warmed to 35±1° C.) was added to the sample and the bag was sealed. The samples were hand mixed by massaging for approximately one minute to homogenize. The samples were incubated at 35±1° C. for 16-20 h. After incubation, 20 μL of the enriched samples was placed into 1980 μL of BHI and incubated for an addition 3-4 h at 37±1° C. Brain Heart Infusion (BHI) broth is recommended as a secondary enrichment media in microbiological testing when it is used to enhance the recovery and growth of fastidious or slow-growing bacteria, following primary enrichment in a non-selective media, such as BPW. This helps to improve the chances of detecting and isolating microorganisms from a sample. The formulation for BHI per liter is 17.5 grams of brain heart infusion solids, 10 grams of tryptose, 2 grams of glucose, 5 grams of sodium chloride, and 2.5 grams of disodium hydrogen phosphate.

Chocolate was aseptically sampled (25 g) and place in a sterile stomacher bag. 225 mL±15 mL of BPW (pre-warmed to 35±1° C.) was added to the sample and the bag was sealed. The sample was hand mixed by massaging or stomaching for approximately one minute to homogenize. The samples were incubated at 35±1° C. for 16-20 h. After incubation, 20 μL of the enriched samples was placed into 1980 μL of BHI and incubated for an addition 3-4 h at 37±1° C.

Leafy greens were aseptically sampled (200 g) and placed in a sterile bag. 450 L±30 mL of pre-warmed EREB was added to the sample and the bag sealed. The sample was hand mixed by massaging or stomaching for approximately one minute to homogenize. The samples were incubated at 42±1° C. for 10-18 h. If the sample lysates were to be pooled, the incubation time was increased to 12-18 h.

Dried hemp flower was aseptically sampled (10 g) and placed into a sterile filtered stomacher bag. 90 mL±10 mL of pre-warmed (37° C.) BPW was added to the sample and the bag sealed. The sample was hand mixed by massaging or stomaching for approximately one minute to homogenize. The samples were incubated at 42±1° C. for 24 h. Table 3 shows example enrichment conditions. Enrichment incubation for Table 3 samples is 42±1° C. for 10-18 hours, unless otherwise indicated.

TABLE 3
EREB Enrichment Media Volumes for Samples

| | SAMPLE SIZE/ | |
|---|---|---|
| | ANALYSIS | MEDIA |
| MATRIX | UNIT | VOLUME |
| Raw Ground Beef | /375 g | 1 L ± 50 mL |
| Raw Beef Excision | /375 g | 1 L ± 50 mL |
| Sample N60 | | |
| Leafy Greens | /200 g | 450 mL ± 30 mL |

*Pooled samples should enrich for 12-18 hours

### Cell Lysis Protocol

After incubation, the following steps were taken for cell lysis. The lysis step is shown in FIG. 1C. One 1.5 mL microcentrifuge tube or equivalent PCR-grade plastic tube was labeled per sample. 400 μL of lysis buffer was aseptically pipetted into each tube. 25 mL of lysis buffer contains 24.875 mL of Tris-EDTA buffer and 125 μL of Triton X-100. For chocolate and hemp flower, 995 μL of lysis buffer was pipetted. Unused lysis buffer was returned to storage (2-8° C.). 5 μL of the sample prepared in the enrichment broth was pipetted into the prepared tube. The tube(s) were capped and vortexed. The closed tubes were heated for 10 minutes at 95±3° C. in the heat block. NOTE: Lid locks were used when heating in microcentrifuge tubes. The closed tubes were removed from the heat block and allowed to cool for 5 minutes at room temperature. Samples were either tested directly in the SIMUL-qPCR Salmonella with GFP Assay or the lysate was held in a refrigerator (2-8° C.) up to 48 hours before proceeding to SIMUL-qPCR Salmonella with GFP Assay.

For samples that were enriched for the minimum time to support pooling: up to 10 samples were pooled immediately after cell lysis. 5 μL aliquots from each lysate were combined in a microcentrifuge tube or equivalent PCR-grade plastic tube (total volume should not exceed 50 μL). The pooled samples were pipetted to mix the samples and used directly in the SIMUL-qPCR Salmonella with GFP Assay.

### Assay Preparation & Set Up

The specificity and efficiency of the primers and probes for simultaneous detection of GFP and target bacteria in Examples 1-5 were optimized for the methods and kits disclosed. The Lyo PCR Master Mix was used for PCR components (buffer, magnesium, Taq polymerase, etc.; Apto-Gen, London, UK, which is hereby incorporated by reference in its entirety). Some important factors for optimizing the specificity and efficiency of the primers and probes include the following. 1) Primer and probe specificity. Specificity was found to be crucial for accurate quantification of the target bacterial and control genes. Non-specific binding of primers and probes can lead to an increase in Cq values, as the signal will be amplified from non-target sequences. 2) Primer and probe efficiency. Probes were designed that have a high melting temperature (Tm) and specificity for the target sequence. This was done by selecting probes with a high GC content and minimal homology to non-target sequences. 3) Primer and probe concentration. The concentration of primers and probes can affect the Cq value. Too little primer or probe will result in a higher Cq value, while too much primer or probe can lead to non-specific amplification and a higher Cq value. 4) Annealing temperature. The annealing temperature used in the reaction can affect the specificity of the primer binding and thus the Cq value. If the annealing temperature was too high or too low, the primers may not bind specifically to the target DNA, resulting in a higher Cq value. 5) POR conditions. The PCR conditions, such as the concentration of magnesium ions, the pH and the thermal cycler used can also affect the Cq value. 6) Template concentration. The amount of template DNA present in the reaction can directly affect the Cq value. A higher template concentration will result in a lower Cq value because more target DNA is present to be amplified. 7) Sample preparation. The quality and quantity of the starting material, and the methods used to extract, purify and quantify the nucleic acid sample can affect the Cq value.

The qPCR step is shown in FIG. 1D. The qPCR set up and data entry on the qPCR instrument was completed prior to transferring samples from the cell lysis protocol. The equipment was prepared by turning on the heating blocks to 95±3° C. as measured by a calibrated thermometer. The qPCR instrument was turned on and a run file from SIMUL-qPCR template was created. The SIMUL-qPCR template contains the required cycle. The SIMUL-qPCR was validated using the MyGo Pro PCR, under the profile tab, choose the “Hold” and “2-Step Amplification” predefined programs. For the “Hold” stage, the temperature was set to 95° C., the Ramp to 5° C./s, and the hold time to 120 seconds. For the “2-Step Amplification”, the number of cycles was set to “40”. For “Step 1”, the temperature was set to 95° C., the Ramp to 5° C./s, and the hold time to 10 seconds. For “Step 2”, the temperature was set to 60° C., the Ramp to 4° C./s, and the hold time to 50 seconds with data acquired during “Step 2”. After removing the aliquot required for lysis, the sample enrichment(s) were returned to the incubator.

The target primer probe sets were then added to the sample(s). The concentrations of the primers in the assay were 400 nM. The concentration of the probe in the FAM channel in the assay was 25 nM. The concentration of the probe in all other channels was 100 nM. After cell lysis and loading, the appropriate target PCR probe sets were selected for each. For example, target one could be the Salmonella 1 PCR probe set with the probe labeled with FAM, target two could be the Salmonella 2 PCR probe set with the probe labeled with CAL560, target three could be the GFP PCR probe set with the probe labeled with CAL 670, and target four could be the IAC primer probe set with the probe labeled with CAL 610.

PCR tubes were arranged according to the run file. Using caution, the caps were removed from the PCR tubes. 20 μL of lysate was pipetted into the sample wells of the PCR test strip, ensuring the pellet is containing the PCR reagents was hydrated. PCR pellets must be hydrated and resealed within 10 minutes after removing the caps from the PCR tubes. The caps were placed onto each tube and pressed down to seal each lid, making sure that each lid was tightly secured before running on the PCR machine. If air bubbles were present, the reaction tubes were carefully flicked until no air bubbles remained. The reaction tubes were briefly spun down in a mini-centrifuge, then loaded into the qPCR instrument and the run started.

### Interpreting Results

For qPCR amplification and detection, forward and reverse primers were designed to hybridize to a unique sequence of the Salmonella genomic DNA. A fluorogenic probe consisting of a DNA probe labeled with a 5′-dye and a 3′-quencher was included in the same reaction mixture. During PCR amplification, the probe is cleaved, and the reporter dye and quencher are separated. The resulting increase in fluorescence can be detected on the real-time PCR instrument. Two unique primer and probe mixtures were present in this assay. The Salmonella targets are detected in the AFD-Blue channel and AFD-Orange channel. The GFP target is detected in the AFD-Purple dye channel. An Internal Amplification Control (IAC) is included for additional quality control and is detected in the AFD-Red dye channel. The analysis step is shown in FIG. 1E.

Once the SIMUL-qPCR Assay run was complete, data was analyzed automatically by the software. The software analyzes any DNA amplification data and displays a Cq value for any sample that amplifies. Amplification in the AFD-Blue channel indicated Salmonella. Amplification in the AFD-Purple Channel indicated presence of the GFP. Only a Cq value that has a typical sigmoidal curve or the beginning of the curve is considered positive for the target (FIG. 1E). When a Cq value was not obtained, the result was negative for the target provided a Cq value was present in the AFD-Red channel for the IAC, which indicates that the PCR reaction ran correctly Table 4 below shows results interpretation.

TABLE 4
Exemplary Assay Results and Interpretation

| | Results | | | |
|---|---|---|---|---|
| | AFD-Blue and/or | AFD- | AFD- | |
| | AFD-Orange | Purple | Red | |
| Workflow | (Salmonella) | (GFP) | (IAC) | Interpretation |
| SIMUL-qPCR | C_{q} | C_{q} | C_{q} | Positive for |
| Salmonella | | | | Salmonella |
| with GFP | | | | that contains GFP |
| Assay | C_{q} | No C_{q} | C_{q} | Positive for |
| | | | | Salmonella |
| | | | | that does not |
| | | | | contain GFP |
| | No C_{q} | No C_{q} | C_{q} | Negative |
| | No C_{q} | No C_{q} | No C_{q} | Indeterminate - |
| | | | | Rerun Assay |

Performance using these methods was found to be comparable to that of the U.S. Department of Agriculture Food Safety and Inspection Service, “Isolation and Identification of Salmonella from Meat, Poultry, Pasteurized Egg, and Catfish Products and Carcass and Environmental Sponges”, Microbiology Laboratory Manual 4.09 (2017); which is hereby incorporated by reference in its entirety, or raw beef trim, raw ground beef, raw ground poultry, RTE cooked poultry, frankfurters, pasteurized liquid eggs and poultry carcass rinses, and Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety, for dry pet food, peanut butter, chocolate, leafy greens, and environmental surface sponges/swabs. The SIMUL-qPCR Salmonella with GFP assay was effective in screening for target Salmonella species in dried hemp flower (≤0.3% THC) at a 10 g test portion size and met the requirements of SMPR 2020.002, “AOAC SMPR® 2020.002 Standard Method Performance Requirements (SMPRs®) for Detection of Salmonella species in Cannabis and Cannabis Products”, which is hereby incorporated by reference in its entirety. Lysates from enriched samples could be tested individually or in a pooled format. In the pooled format, up to 10 lysates could be combined. The pooled format was not indicated for pasteurized liquid egg.

### Results of Matrix Study of Various Food Types

Dry pet food, chocolate, and leafy greens were obtained from a local grocery store. Dried hemp flower was obtained from a hemp producer in Oregon. For dry pet food and chocolate, the matrixes were prescreened for Salmonella using the SIMUL-qPCR Salmonella assay to determine if any natural contamination was present. Leafy greens and dried hemp flower were prescreened for both the Top 7 STEC strains and for Salmonella using the SIMUL-qPCR Salmonella and Top 7 STEC assays prior to testing. Note: Because both EREB and BPW media is designed to enrich for both STEC and Salmonella, leafy greens and dried hemp flower were co-inoculated with a STEC strain and a Salmonella strain and then tested by the SIMUL-qPCR E. coli O157:H7 with GFP and SIMUL-qPCR Salmonella with GFP Assays. The results of the STEC testing by the SIMUL-qPCR E. coli O157:H7 with GFP Assay are presented in Example 3.

To prepare the materials for testing, each food matrix was aseptically combined into a large sterile sampling bag and hand massaged to thoroughly mix the product. The product was spiked using strains sub-cultured in BHI at 37±2° C. for 20-24 hrs. All four matrixes were inoculated with S. Typhimurium Sal54 derived from SAL5694 as that strain included the GFP target. Leafy greens and dried hemp flower were co-inoculated with E. coli O157:H7 EC43 derived from FDA ESC1177 (STEC).

Each matrix was artificially contaminated with the indicated strains at two contamination levels; a low level to achieve a fractional response of 5-15 positive test portions out of 20 replicate portions tested (approximately 0.2-2 cfu/25 g), and a high level to achieve five positive test portions out of five replicate portions tested (approximately 5-10 cfu/25 g). Samples were mixed well to create homogenous mixtures.

For dry pet food, the strain was heat stressed before spiking. The liquid culture was heated to 55° C. for 10-20 min to achieve 50-80% injury. Heat stressed strain was plated onto XLT4 agar and Standard Methods Agar (SMA) to determine the percent injury.

For dry dog food and chocolate, 25 g test portions of each contamination level (including the uncontaminated level) were weighed into a sterile bag. The portions were kept at room temperature for two weeks to allow for stabilization. For leafy greens, 25 g test portions of each contamination level (including the uncontaminated level) were weighed into a sterile bag and held at 2-8° C. for 48-72 h. For dried hemp flower, 10 g test portions of each contamination level (including the uncontaminated level) were weighed into a sterile filtered stomacher bag and held at room temperature (20-25° C.) for 2 weeks. Each matrix was also tested for aerobic plate count. See Maturin and Peeler, “BAM Chapter 3: Aerobic Plate Count,” U.S. Food and Drug Administration Bacteriological Analytical Manual, which is hereby incorporated by reference in its entirety.

For all matrixes except dried hemp flower, the BAM method was used as the reference method. For the dried hemp flower, the SMPR 2020.002 “Detection of Salmonella species in Cannabis and Cannabis Products”, which is hereby incorporated by reference in its entirety, was followed. For dry pet food (dry dog food), a 25 g sample was added to a sterile blending container. A 1:10 dilution using lactose broth was obtained and then blended for 2 min. The mixture was then transferred to another sterile container and left out for one hour±5 min at room temperature with the jar securely capped. After swirling the mixture, the pH was then determined and adjusted to 6.8±2. After pH adjustment, Triton X-100 steamed for 15 min was added. The sample was then incubated at 35° C. for 24±2 h with the cap loosened ¼ turn. For chocolate, a 25 g sample was combined with 225 mL of reconstituted nonfat dry milk and blended for 2 min. After blending, the mixture was left at room temperature (20-25° C.) for 60±5 min securely capped before adjusting the pH to 6.8±2. Then 0.45 mL of 1% aqueous brilliant green dry solution was added to the sample. The sample was mixed well and then incubated at 35±2° C. for 24±2 h with the cap loosed ¼ turn. For leafy greens, a 25 g sample was added to a sterile container. Universal Pre-enrichment broth, 225 mL, was added without homogenization. The sample was incubated at 35±2° C. for 24±2 h. For dried hemp flower, there was no reference method enrichment, and so only the method enrichment described above was performed.

For the SIMUL-qPCR Salmonella with GFP Assay, each matrix was enriched as described above. For leafy greens, an additional 175 g of inoculated product was added to each sterile bag to bring the weight up to 200 g. After incubation, each matrix was tested as individual portions and in a pooled format. To prepare the pooled format, test portions were lysed, then 5 μL from each test portion lysate was combined with 5 μL from each of 9 non-inoculated test portion lysates (presumably negative) to create a pool of 10 portions (total volume 50 μL). A pooled sample was prepared to represent each test portion from each level. For each matrix except the dried hemp flower, an unpaired study was conducted due to the difference in enrichment media and incubation times between the candidate and reference methods. Samples were processed using either the SIMUL-qPCR Salmonella with GFP Assay or the BAM Chapter 5 method (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety). For the dried hemp flower, a paired study was conducted.

All samples were run on the SIMUL-qPCR Salmonella with GFP Assay and confirmed following the BAM Chapter 5 method (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety). All enrichments, including test portions that were not inoculated with Salmonella, were processed for cultural confirmation regardless of the results obtained by the SIMUL-qPCR method. Colonies were confirmed using the API 20E strips along with group O and poly H antisera for biochemical and serological confirmations. For all food matrixes, Most Probable Number (“MPN”) analyses were conducted on the high and low contamination levels. Test portions from the reference method analysis, plus additional portions from each contamination level were used to create a minimum 5 replicate, 3-level MPN following the BAM Chapter 5 protocol (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety). MPN results were calculated using the MPN v1.6 calculator program (Least Cost Formulations, Virginia Beach, VA).

All test results were analyzed by probability of detection (POD) statistical analysis using the AOAC Binary Workbook v5.1 (Least Cost Formulations, Virginia Beach, VA), which is hereby incorporated by reference in its entirety. The paired POD analysis was used to determine presumptive vs confirmed results, and the unpaired analysis was used to compare the candidate method results to the reference method results. The 95% confidence interval on the difference between two POD values must contain 0 for results to be considered not statistically different. The matrix study results are detailed in Tables 5-12.

Three lots of SIMUL-qPCR Salmonella with GFP Assay Kit were compared to a lot of SIMUL-qPCR Salmonella Assay kit for lot-to-lot variability. The lot numbers of the three kits containing the GFP target were 26321, 35621, and 00322. The lot of the original kit without the GFP target was 31221.

A pure culture of UV-BioTAG™ Salmonella (Salmonella enterica subsp. Enterica serovar Typhimurium Sal54 derived from SAL5694) was grown in BPW media at 35±1° C. for 16-20 h. The culture was diluted in BPW to a level that yielded fractional results. A pure culture of Citrobacter freundii (SGSC 5346) was also grown up in BHI at 37° C. for 20-24 h. This culture was not diluted. Each lot of assays were tested with 10 replicates of diluted Salmonella tagged with GFP and 10 replicates of undiluted Citrobacter. The samples were blind coded and randomized before being tested.

Results of the matrix study are outlined in Tables 5-12. Some challenges were encountered when trying to achieve fractional levels of chocolate. It was during experimentation that it was realized a regrow step along with a larger lysis volume was necessary. It was also determined that the assay performed well on dry dog food with the regrow step, which led to the inclusion of that step for that matrix. Also, during confirmation testing, some enriched test portions had to be re-streaked to remove some of the competing background flora present in the enrichments. For dried hemp flower, it was determined through different trials that the media should be pre-warmed at 37° C. and the samples should be incubated at 42° C. instead of 35° C. The background flora for each matrix, determined by aerobic plate count (see Maturin and Peeler, “BAM Chapter 3: Aerobic Plate Count,” U.S. Food and Drug Administration Bacteriological Analytical Manual, which is hereby incorporated by reference in its entirety), was 1520 cfu/g for pet food, 50 cfu/g for chocolate, 340 cfu/g for leafy greens, and 20 cfu/g for hemp. The results in Tables 5-12 include the probability of detection (POD), which is the proportion of positive analytical outcomes for a qualitative method for a given matrix at a given analyte level or concentration. POD is concentration dependent. Several POD measures can be calculated; POD_{R} (reference method POD), POD_{C} (confirmed candidate method POD), POD_{CP} (candidate method presumptive result POD) and POD_{CC} (candidate method confirmation result POD). The difference of probabilities of detection (dPOD) is the difference between any two POD values. If the confidence interval of a dPOD does not contain zero, then the difference is statistically significant at the 5% level.

PCR results were analyzed for both the Salmonella target as well as the GFP target. For all results listed, both targets called positive to be included in the number of positives displayed in the results tables. For confirmation of the candidate method, colonies were viewed under UV light to confirm the presence of the GFP. All samples that confirmed positive for Salmonella also confirmed for the presence of GFP.

For dry pet food, 10 out of 20 test portions were presumptive positive for the candidate method for both the individual and pooled lysates. All 10 portions confirmed positive. For the reference method, 9 test portions were positive.

For chocolate, 11 out of 20 test portions were presumptive positive for both the individual and pooled lysates, and 12 out of 20 confirmed positive. Thirteen out of 20 test portions were positive for the reference method.

For leafy greens, 6 out of 20 test portions were presumptive positive for both the individual and pooled lysates. Six out of 20 portions were positive for both the reference and candidate methods.

For dried hemp flower, 14 out of 20 test portions were presumptive positive for the individual lysates and 15 out of 20 test portions were presumptive positive for the pooled lysates. Fifteen out of 20 samples confirmed positive for the candidate method.

For all matrixes for both the individual and pooled lysates, no statistically significant differences were found. For all portions analyzed, the GFP target also amplified when the Salmonella target amplified, demonstrating the assay's ability to detect both Salmonella as well as the GFP target.

Conclusion—These data confirmed that the SIMUL-qPCR Salmonella with GFP Assay was suitable for detecting Salmonella species in dry pet food, chocolate, leafy greens, and dried hemp flower. Furthermore, the data confirmed that the method was valid for pooled lysates. POD analysis showed that there were no statistically significant differences between the candidate method and the BAM Chapter 5 reference method for any of the matrixes (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety). The study also demonstrated that the assay could yield consistent results compared to the assay that did not contain the GFP target. Finally, the study proved that the assay could detect a GFP target if a Salmonella strain was tagged with the GFP, which can add value to laboratories in limiting time and labor in investigations into possible internal cross-contamination issues.

TABLE 5
SIMUL-qPCR Salmonella with GFP Assay Presumptive vs. Confirmed Results -
Individual lysates for leafy greens and dried hemp flower

| | | | | SIMUL-qPCR Salmonella | | | SIMUL-qPCR Salmonella | | | | |
|---|---|---|---|---|---|---|---|---|---|---|---|
| | | | | with GFP Presumptive | | | with GFP Confirmed | | | | |
| Matrix | Strain | MPN^{a}/25 g | N^{b} | x^{c} | POD_{CP}^{d} | 95% CI | x | POD_{CC}^{e} | 95% CI | dPOD_{CP}^{f} | 95% CI^{g} |
| Leafy | Salmonella | N/Aⁱ | 5 | 0 | 0.00 | 0.00, 0.43 | 0 | 0.00 | 0.00, 0.43 | 0.00 | −0.43, 0.43 |
| Greens | Typhimurium | 1.097 (0.69, 1.86) | 20 | 6 | 0.30 | 0.15, 0.52 | 5 | 0.30 | 0.15, 0.52 | 0.00 | −0.27, 0.27 |
| 10 h | FDA | 1E+12 | 5 | 5 | 1.00 | 0.57, 1.00 | 5 | 1.00 | 0.57, 1.00 | 0.00 | −0.43, 0.43 |
| | SAL54^{h} | (4.73, 1E+12)^{j} | | | | | | | | | |
| Leafy | Salmonella | N/A | 5 | 0 | 0.00 | 0.00, 0.43 | 0 | 0.00 | 0.00, 0.43 | 0.00 | −0.43, 0.43 |
| Greens | Typhimurium | 1.097 (0.69, 1.86) | 20 | 6 | 0.30 | 0.15, 0.52 | 5 | 0.30 | 0.15, 0.52 | 0.00 | −0.27, 0.27 |
| 18 h | FDA | 1E+12 | 5 | 5 | 1.00 | 0.57, 1.00 | 5 | 1.00 | 0.57, 1.00 | 0.00 | −0.43, 0.43 |
| | SAL54 | (4.73, 1E+12) | | | | | | | | | |
| Dried | Salmonella | N/A | 5 | 0 | 0.00 | 0.00, 0.43 | 0 | 0.00 | 0.00, 0.43 | 0.00 | −0.43, 0.43 |
| Hemp | Typhimurium | 1.19 (0.73, 2.09) | 20 | 14 | 0.70 | 0.48, 0.75 | 15 | 0.75 | 0.53, 0.89 | −0.05 | −0.31, 0.22 |
| Flower | FDA | 1E+12 | 5 | 5 | 1.00 | 0.57, 1.00 | 5 | 1.00 | 0.57, 1.00 | 0.00 | −0.43, 0.43 |
| 24 h | SAL54 | (8.18, 1E+12) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}POD_{CP} = Difference between the candidate method presumptive result and candidate method confirmed result POD values.
^{g}995% CI = If the confidence interval of a dPOD does not contain zero, then the difference is statistically significant at the 5% level.
^{h}Strain derived from FDA SAL5694, Food and Drug Administration, Silver Spring, MD.
ⁱN/A = Not applicable.
^{j}All test portions in the MPN were positive, leading to the high value.

TABLE 6
SIMUL-qPCR Salmonella with GFP Assay vs. Reference Method Results - Individual lysates for leafy greens

| | | | | SIMUL-qPCR Salmonella | | | | | | | |
|---|---|---|---|---|---|---|---|---|---|---|---|
| | | | | with GFP Confirmed | | | BAM Ch. 5 | | | | |
| Matrix | Strain | MPN^{a}/25 g | N^{b} | x^{c} | POD_{C}^{d} | 95% CI | x | POD_{R}^{e} | 95% CI | dPOD_{C}^{f} | 95% CI^{g} |
| Leafy | Salmonella | NAⁱ | 5 | 0 | 0.00 | 0.00, 0.43 | 0 | 0.00 | 0.00, 0.43 | 0.00 | −0.43, 0.43 |
| Greens | Typhimurium | 1.097 | 20 | 6 | 0.30 | 0.15, 0.52 | 6 | 0.30 | 0.15, 0.52 | 0.00 | −0.27, 0.27 |
| 10 h | FDA SAL54^{h} | (0.69, 1.86) | | | | | | | | | |
| | | 1E+12 | 5 | 5 | 1.00 | 0.57, 1.00 | 5 | 1.00 | 0.57, 1.00 | 0.00 | −0.43, 0.43 |
| | | (4.73, 1E+12)ⁱ | | | | | | | | | |
| Leafy | Salmonella | N/A | 5 | 0 | 0.00 | 0.00, 0.43 | 0 | 0.00 | 0.00, 0.43 | 0.00 | −0.43, 0.43 |
| Greens | Typhimurium | 1.097 | 20 | 6 | 0.30 | 0.15, 0.52 | 6 | 0.30 | 0.15, 0.52 | 0.00 | −0.27, 0.27 |
| 18 h | FDA SAL54 | (0.69, 1.86) | | | | | | | | | |
| | | 1E+12 | 5 | 5 | 1.00 | 0.57, 1.00 | 5 | 1.00 | 0.57, 1.00 | 0.00 | −0.43, 0.43 |
| | | (4.73, 1E+12) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method positive result and reference method result POD values.
^{g}95% CI = If the confidence interval of a dPOD does not contain zero, then the difference is statistically significant at the 5% level.
^{h}Strain derived from FDA SAL5694, Food and Drug Administration, Silver Spring, MD.
ⁱN/A = Not applicable.
^{j}All test portions in the MPN were positive, leading to the high value.

TABLE 7
SIMUL-qPCR Salmonella with GFP Assay Presumptive vs. Confirmed
Results - Pooled lysates for leafy greens and dried hemp flower

| | | | | SIMUL-qPCR Salmonella | | | SIMUL-qPCR Salmonella | | | | |
|---|---|---|---|---|---|---|---|---|---|---|---|
| | | | | with GFP Presumptive | | | with GFP Confirmed | | | | |
| Matrix | Strain | MPN^{a}/25 g | N^{b} | x^{c} | POD_{CP}^{d} | 95% CI | x^{c} | POD_{CC}^{e} | 95% CI | dPOD_{CP}^{f} | 95% CI^{g} |
| Leafy | Salmonella | N/Aⁱ | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 12 h | FDA SAL54^{h} | 1.097 | 20 | 6 | 0.30 | 0.15, 0.52 | 6 | 0.30 | 0.15, 0.52 | 0.00 | −0.27, |
| | | (0.69, 1.86) | | | | | | | | | 0.27 |
| | | 1E+12 (4.73, | 5 | 5 | 1.00 | 0.57, 1.00 | 5 | 1.00 | 0.57, 1.00 | 0.00 | −0.43, |
| | | 1E+12)^{j} | | | | | | | | | 0.43 |
| Leafy | Salmonella | N/A | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 18 h | FDA SAL54 | 1.097 (0.69, | 20 | 6 | 0.30 | 0.15, 0.52 | 6 | 0.30 | 0.15, 0.52 | 0.00 | −0.27, |
| | | 1.86) | | | | | | | | | 0.27 |
| | | 1E+12 (4.73, | 5 | 5 | 1.00 | 0.57, 1.00 | 5 | 1.00 | 0.57, 1.00 | 0.00 | −0.43, |
| | | 1E+12) | | | | | | | | | 0.43 |
| Dried | Salmonella | N/A | 5 | 0 | 0.00 | 0.00, 0.43 | 0 | 0.00 | 0.00, 0.43 | 0.00 | −0.43, |
| Hemp | Typhimurium | | | | | | | | | | 0.43 |
| Flower | FDA SAL54 | 1.19 (0.73, | 20 | 15 | 0.75 | 0.53, 0.89 | 15 | 0.75 | 0.53, 0.89 | 0.00 | −0.26, |
| 24 h | | 2.09) | | | | | | | | | 0.26 |
| | | 1E+12 (8.18, | 5 | 5 | 1.00 | 0.57, 1.00 | 5 | 1.00 | 0.57, 1.00 | 0.00 | −0.43, |
| | | 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values
^{g}95% CI = If the confidence interval of a dPOD does not contain zero, then the difference is statistically significant at the 5% level.
^{h}Strain derived from FDA SAL5694, Food and Drug Administration, Silver Spring, MD.
ⁱN/A = Not applicable;
^{j}All test portions in the MPN were positive, leading to the high value.

TABLE 8
SIMUL-qPCR Salmonella with GFP Assay vs. Reference Method Results - Pooled lysates for leafy greens

| | | | | SIMUL-qPCR Salmonella | | | | | | | |
|---|---|---|---|---|---|---|---|---|---|---|---|
| | | | | with GFP Confirmed | | | BAM Ch. 5 | | | | |
| Matrix | Strain | MPN^{a}/25 g | N^{b} | x^{c} | POD_{C}^{d} | 95% CI | x^{c} | POD_{R}^{e} | 95% CI | dPOD_{C}^{f} | 95% CI^{g} |
| Leafy | Salmonella | N/Aⁱ | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 12 h | FDA SAL54^{h} | 1.097 (0.69, | 20 | 6 | 0.30 | 0.15, 0.52 | 6 | 0.30 | 0.15, 0.52 | 0.00 | −0.27, |
| | | 1.86) | | | | | | | | | 0.27 |
| | | 1E+12 (4.73, | 5 | 5 | 1.00 | 0.57, 1.00 | 5 | 1.00 | 0.57, 1.00 | 0.00 | −0.43, |
| | | 1E+12)^{j} | | | | | | | | | 0.43 |
| Leafy | Salmonella | N/A | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 18 h | FDA SAL54 | 1.097 (0.69, | 20 | 6 | 0.30 | 0.15, 0.52 | 6 | 0.30 | 0.15, 0.52 | 0.00 | −0.27, |
| | | 1.86) | | | | | | | | | 0.27 |
| | | 1E+12 (4.73, | 5 | 5 | 1.00 | 0.57, 1.00 | 5 | 1.00 | 0.57, 1.00 | 0.00 | −0.43, |
| | | 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
dPOD_{C} = Candidate method presumptive positive outcomes divided by the total number of trials.
^{e}POD_{R} = Reference method positive outcomes divided by the total number of trials.
^{f}dPOD_{C} = Difference between the candidate method positive result and reference method result POD values
^{g}95% CI = If the confidence interval of a dPOD does not contain zero, then the difference is statistically significant at the 5% level.
^{h}Strain derived from FDA SAL5694, Food and Drug Administration, Silver Spring, MD.
ⁱN/A = Not applicable;
^{j}All test portions in the MPN were positive, leading to the high value.

TABLE 9
SIMUL-qPCR Salmonella with GFP Presumptive vs. Confirmed Results -
Individual lysates for dry pet food and chocolate

| | | | | | | | SIMUL-qPCR | | | | |
|---|---|---|---|---|---|---|---|---|---|---|---|
| | | | | SIMUL-qPCR Salmonella | | | Salmonella | | | | |
| | | | | with GFP presumptive | | | with GFP confirmed | | | | |
| Matrix | Strain | MPN^{a}/25 g | N^{b} | x^{c} | POD_{CP}^{d} | 95% CI | x | POD_{CC}^{e} | 95% CI | dPOD_{CP}^{f} | 95% CI^{g} |
| Dry Pet | Salmonella | N/Aⁱ | 5 | 0 | 0.00 | 0.00, 0.43 | 0 | 0.00 | 0.00, 0.43 | 0.00 | −0.43, 0.43 |
| Food | Typhimurium | 1.40 (0.91, | 20 | 10 | 0.50 | 0.30, 0.70 | 10 | 0.50 | 0.30, 0.70 | 0.00 | −0.28, 0.28 |
| 25 g | FDA SAL54^{h} | 2.40) | | | | | | | | | |
| | | 6.45 (2.88, | 5 | 5 | 1.0 | 0.57, 1.00 | 5 | 1.00 | 0.57, 1.00 | 0.00 | −0.43, 0.43 |
| | | 14.45) | | | | | | | | | |
| Chocolate | Salmonella | N/A | 5 | 0 | 0.00 | 0.00, 0.43 | 0 | 0.00 | 0.00, 0.43 | 0.00 | −0.43, 0.43 |
| 25 g | Typhimurium | 1.11 (0.62, | 20 | 11 | 0.55 | 0.34, 0.74 | 12 | 0.60 | 0.39, 0.78 | −0.05 | −0.33, 0.24 |
| | FDA SAL54 | 1.69) | | | | | | | | | |
| | | 1E+12 (5.07, | 5 | 5 | 1.00 | 0.57, 1.00 | 5 | 1.00 | 0.57, 1.00 | 0.00 | −0.43, 0.43 |
| | | 1E+12) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values
^{g}95% CI = If the confidence interval of a dPOD does not contain zero, then the difference is statistically significant at the 5% level.
^{h}FDA = Strain derived from FDA SAL5694, Food and Drug Administration, Silver Spring, MD.
ⁱN/A = Not applicable;
^{j}All test portions in the MPN were positive, leading to the high value.

TABLE 10
SIMUL-qPCR Salmonella with GFP Assay vs. Reference Method Results -
Individual lysates for dry pet food and chocolate

| | | | | SIMUL-qPCR Salmonella | | | | | | | |
|---|---|---|---|---|---|---|---|---|---|---|---|
| | | | | with GFP presumptive | | | BAM Ch. 5 | | | | |
| Matrix | Strain | MPN^{a}/25 g | N^{b} | x^{c} | POD_{C}^{d} | 95% CI | x | POD_{R}^{e} | 95% CI | dPOD_{C}^{f} | 95% CI^{g} |
| Dry Pet | Salmonella | N/Aⁱ | 5 | 0 | 0.00 | 0.00, 0.43 | 0 | 0.00 | 0.00, 0.43 | 0.00 | −0.43, 0.43 |
| Food | Typhimurium | 1.40 (0.91, | 20 | 10 | 0.50 | 0.30, 0.70 | 9 | 0.45 | 0.26, 0.66 | 0.05 | −0.24, 0.33 |
| 25 g | FDA SAL54^{h} | 2.40) | | | | | | | | | |
| | | 6.45 (2.88, | 5 | 5 | 1.0 | 0.57, 1.00 | 5 | 1.00 | 0.57, 1.00 | 0.00 | −0.43, 0.43 |
| | | 14.45) | | | | | | | | | |
| Chocolate | Salmonella | N/A | 5 | 0 | 0.00 | 0.00, 0.43 | 0 | 0.00 | 0.00, 0.43 | 0.00 | −0.43, 0.43 |
| 25 g | Typhimurium | 1.11 (0.62, | 20 | 11 | 0.55 | 0.34, 0.74 | 13 | 0.65 | 0.43, 0.82 | −0.10 | −0.37, 0.19 |
| | FDA SAL54 | 1.69) | | | | | | | | | |
| | | 1E+12 (5.07, | 5 | 5 | 1.00 | 0.57, 1.00 | 5 | 1.00 | 0.57, 1.00 | 0.00 | −0.43, 0.43 |
| | | 1E+12)^{j} | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method confirmed positive outcomes divided by the total number of trials.
^{f}dPOD_{C} = Difference between the candidate method positive result and reference method result POD values
^{g}95% CI = If the confidence interval of a dPOD does not contain zero, then the difference is statistically significant at the 5% level.
^{h}FDA = Strain derived from FDA SAL5694, Food and Drug Administration, Silver Spring, MD.
ⁱN/A = Not applicable;
^{j}All test portions in the MPN were positive, leading to the high value.

TABLE 11
SIMUL-qPCR Salmonella with GFP Presumptive vs. Confirmed Results -
Pooled lysates for dry pet food and chocolate

| | | | | | | | SIMUL-qPCR | | | | |
|---|---|---|---|---|---|---|---|---|---|---|---|
| | | | | SIMUL-qPCR Salmonella | | | Salmonella with GFP | | | | |
| | | | | with GFP presumptive | | | confirmed | | | | |
| Matrix | Strain | MPN^{a}/25 g | N^{b} | x^{c} | POD_{CP}^{d} | 95% CI | x | POD_{CC}^{e} | 95% CI | dPOD_{CP}^{f} | 95% CI^{g} |
| Dry Pet | Salmonella | N/Aⁱ | 5 | 0 | 0.00 | 0.00, 0.43 | 0 | 0.00 | 0.00, 0.43 | 0.00 | −0.43, 0.43 |
| Food | Typhimurium | 1.40 (0.91, | 20 | 10 | 0.50 | 0.30, 0.70 | 10 | 0.50 | 0.30, 0.70 | 0.00 | −0.28, 0.28 |
| 25 g | FDA SAL54^{h} | 2.40) | | | | | | | | | |
| | | 6.45 (2.88, | 5 | 5 | 1.0 | 0.57, 1.00 | 5 | 1.00 | 0.57, 1.00 | 0.00 | −0.43, 0.43 |
| | | 14.45) | | | | | | | | | |
| Chocolate | Salmonella | N/A | 5 | 0 | 0.00 | 0.00, 0.43 | 0 | 0.00 | 0.00, 0.43 | 0.00 | −0.43, 0.43 |
| 25 g | Typhimurium | 1.11 (0.62, | 20 | 11 | 0.55 | 0.34, 0.74 | 12 | 0.60 | 0.39, 0.78 | −0.05 | −0.33, 0.24 |
| | FDA SAL54 | 1.69) | | | | | | | | | |
| | | 1E+12 (5.07, | 5 | 5 | 1.00 | 0.57, 1.00 | 5 | 1.00 | 0.57, 1.00 | 0.00 | −0.43, 0.43 |
| | | 1E+12) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values
^{g}95% CI = If the confidence interval of a dPOD does not contain zero, then the difference is statistically significant at the 5% level.
^{h}FDA = Strain derived from FDA SAL5694, Food and Drug Administration, Silver Spring, MD.
ⁱN/A = Not applicable;
^{j}All test portions in the MPN were positive, leading to the high value.

TABLE 12
SIMUL-qPCR Salmonella with GFP Assay vs. Reference Method
Results -Pooled lysates for dry pet food and chocolate

| | | | | SIMUL-qPCR Salmonella | | | | | | | |
|---|---|---|---|---|---|---|---|---|---|---|---|
| | | | | with GFP presumptive | | | BAM Ch. 5 | | | | |
| Matrix | Strain | MPN^{a}/25 g | N^{b} | x^{c} | POD_{C}^{d} | 95% CI | x | POD_{R}^{e} | 95% CI | dPOD_{C}^{f} | 95% CI^{g} |
| Dry Pet | Salmonella | N/Aⁱ | 5 | 0 | 0.00 | 0.00, 0.43 | 0 | 0.00 | 0.00, 0.43 | 0.00 | −0.43, 0.43 |
| Food | Typhimurium | 1.40 (0.91, | 20 | 10 | 0.50 | 0.30, 0.70 | 9 | 0.45 | 0.26, 0.66 | 0.05 | −0.24, 0.33 |
| 25 g | FDA SAL54^{h} | 2.40) | | | | | | | | | |
| | | 6.45 (2.88, | 5 | 5 | 1.0 | 0.57, 1.00 | 5 | 1.00 | 0.57, 1.00 | 0.00 | −0.43, 0.43 |
| | | 14.45) | | | | | | | | | |
| Chocolate | Salmonella | N/A | 5 | 0 | 0.00 | 0.00, 0.43 | 0 | 0.00 | 0.00, 0.43 | 0.00 | −0.43, 0.43 |
| 25 g | Typhimurium | 1.11 (0.62, | 20 | 11 | 0.55 | 0.34, 0.74 | 13 | 0.65 | 0.43, 0.82 | −0.10 | −0.37, 0.19 |
| | FDA SAL54 | 1.69) | | | | | | | | | |
| | | 1E+12 (5.07, | 5 | 5 | 1.00 | 0.57, 1.00 | 5 | 1.00 | 0.57, 1.00 | 0.00 | −0.43, 0.43 |
| | | 1E+12) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
ePOD_{R} = Reference method positive outcomes divided by the total number of trials.
^{f}dPOD_{C} = Difference between the candidate method positive result and reference method result POD values
^{g}95% CI = If the confidence interval of a dPOD does not contain zero, then the difference is statistically significant at the 5% level.
^{h}FDA = Strain derived from FDA SAL5694, Food and Drug Administration, Silver Spring, MD.
ⁱN/A = Not applicable;
^{j}All test portions in the MPN were positive, leading to the high value.

### Example 2—Listeria with GFP Assay

A protocol was developed as a multifaceted approach to the detection of Listeria species in a variety of food products and environmental samples. The workflow of this protocol is illustrated in FIGS. 1A-E. A specifically formulated media was utilized for enriching samples followed by cultural (detection plate) and rapid (qPCR) detection procedures. Listeria Recovery and Enrichment Broth (LREB) combined nutritional components with additional ingredients that were determined to be necessary to selectively improve recovery and growth of Listeria. The selective agents present in LREB were optimized to efficiently inhibit competing normal bacterial flora without affecting the growth of Listeria species. LREB was formulated for buffering capacity to ensure growth in a variety of matrices.

The SIMUL-qPCR Listeria species and monocytogenes with GFP Assay incorporated the detection of a Green Fluorescent Protein (GFP). Listeria does not naturally contain GFP. By utilizing a Listeria quality control strain tagged with GFP, it can be determined if any potential positives were caused by cross-contamination. Previously, potential positives had to be streaked for cultural confirmation and then the colonies would need to be examined under a UV light with the appropriate wavelength, adding days to the process of determining whether any cross-contamination had occurred. With this assay, it could be determined by the end of the PCR run. The test sample and control sample (strain engineered with GFP) are shown in FIG. 1A.

The Simultaneous Multiplex Real Time PCR (SIMUL-qPCR) Listeria species and monocytogenes with GFP Assay was validated for a variety of foods and environmental samples. USDA FSIS and FDA BAM methods were used for method comparison testing. See U.S. Department of Agriculture Food Safety and Inspection Service, “Microbiology Laboratory Manual, Isolation and Identification of Listeria monocytogenes from Red Meat, Poultry and Egg Products, and Environmental Samples,” 8.10 (2017); U.S. Food and Drug Administration, “Detection and Enumeration of Listeria monocytogenes in Foods,” Bacteriological Analytical Manual Chapter 10 (2017), which are hereby incorporated by reference in their entirety. The SIMUL-qPCR Listeria species and monocytogenes with GFP Assay was found to be equivalent to the reference methods. The limit of detection for this SIMUL-qPCR assay was determined to be 1,000 CFU/mL of enriched sample.

### Selective Enrichment Media

Enrichment Ready Media: One liter of LREB was prepared using 17 grams of tryptone, 3 grams of soytone, 6 grams of yeast extract, 2.5 grams of dextrose, 2.5 grams of sodium chloride, 5 grams of lithium chloride, 1 gram of sodium pyruvate, 0.005 grams of acriflavin, and 0.01 grams of naldixic acid. The enrichment step is shown in FIG. 1B. Enrichment included preparing a sample bag with pre-warmed (35±1° C.) laboratory grade ASTM D1193 Type 2 water with the volume respective of the pouch size, which wasused within three to four hours while maintaining pre-warmed conditions (35±1° C.).

### Enrichment for Environmental Surface Sample

Sponge: When ready to test, the prepared LREB was pre-warmed to 35±1° C. and the pre-warmed LREB was added to each sponge sample. Enrichment of Samples Table 13 shows acceptable media volumes by sample type. The sample was homogenized for 30 seconds in a stomacher/blender or equivalent. Hand mixing is an acceptable alternative for stomaching. To hand mix, each sponge that is in the sealed bag Refer to Enrichment of Samples Table 13 shows enrichment conditions. Enrichment Incubation was 30±1° C. for 30-36 hours, unless otherwise indicated.

Swab: When ready to test, the prepared LREB was warmed to 35±1° C., and 9 mL of the pre-warmed LREB was added to each swab sample in its vial. The sample was incubated. The Enrichment of Samples Table 13 shows various enrichment conditions. The samples were incubated at 30±1° C. for 30-36 h.

TABLE 13
LREB Enrichment Media Volumes for Samples

| | SAMPLE SIZE/ | | |
|---|---|---|---|
| MATRIX | ANALYSIS UNIT | | MEDIA VOLUME |
| Environmental Swab* | /Swab | | 9 mL ± 1 mL |
| AFD MEMP Reserve Swab* | /Swab | | 9 mL ± 1 mL |
| Environmental Sponge* | /Sponge | | 90 mL ± 10 mL |
| Frankfurter | /125 | g | 1 L ± 50 mL |
| RTE Sliced Turkey | /125 | g | 1 L ± 50 mL |
| Soft Fresh Cheese (Raw) | /25 | g | 225 mL ± 15 mL |
| Chicken Salad | /25 | g | 225 mL ± 15 mL |
| Ice Cream | /25 | g | 225 mL ± 15 mL |
| Cooked Eggs | /25 | g | 225 mL ± 15 mL |
| Pasteurized Milk | /25 | g | 225 mL ± 15 mL |
| Frozen/Cooked Shrimp | /25 | g | 225 mL ± 15 mL |
| Leafy Greens | /25 | g | 225 mL ± 15 mL |

*Validated surfaces were stainless steel, plastic, rubber, ceramic tile, and sealed concrete

### Enrichment for Food Sample

Food Sample: In general, food products were aseptically sampled and placed in a sterile bag. When ready to be tested, the prepared LREB medium was pre-warmed to 35±1° C. The pre-warmed LREB was then added to the sample according to sample type as outlined in Table 13. The sample was then homogenized for 30 seconds in a stomacher/blender or equivalent. Hand mixing is an acceptable alternative as described above. The sample were incubated as indicated in Table 13 for enrichment conditions.

For example, 125 g of frankfurters or RTE sliced turkey were aseptically sampled and place in a sterile bag. 1 L±50 mL of pre-warmed LREB was added to the sample(s). The samples were hand mixed by massaging each sample in the sealed bag for approximately one minute to homogenize each sample. The samples were incubated at 30° C.±1° C. for 30-36 h.

25 g of soft fresh raw cheese, chicken salad, ice cream, cooked eggs, pasteurized milk, frozen/cooked shrimp, and leafy greens were aseptically sampled and placed in sterile bags. 225 mL±15 mL of pre-warmed LREB was added to the sample bags. The samples were hand mixed by massaging or stomaching each sample that was in the sealed bag for approximately one minute to homogenize each sample. The samples were incubated at 30±1° C. for 30-36 h.

### Cell Lysis Protocol

Cell lysis was performed as described in Example 1. The lysis step is shown in FIG. 1C. After lysis, Samples were either tested directly in the SIMUL-qPCR Listeria Assay or the lysate was held in a refrigerator (2-8° C.) up to 48 hours before proceeding to SIMUL-qPCR Listeria Assay.

Up to 10 samples could be pooled immediately after cell lysis. For pooling, 5 μL aliquots from each lysate were combined in a microcentrifuge tube or equivalent PCR-grade plastic tube (total volume did not exceed 50 μL). The pooled samples were pipetted to mix the samples and used directly in the SIMUL-qPCR Listeria Assay. The pooling process is AOAC PTM approved for leafy greens.

### Assay Preparation & Set Up

The qPCR set up and data entry was set up as described in Example 1, and is shown in FIG. 1D. To test for Listeria species, the target was selected for Listeria species. To test for Listeria monocytogenes, the target was selected for Listeria monocytogenes. To test for both Listeria species and monocytogenes, both targets were selected. The GFP target was added to all runs. After removing the aliquot required for lysis, return the enrichment(s) to the incubator.

### Interpreting Results

For qPCR amplification and detection, forward and reverse primers hybridize to unique sequences of Listeria species and monocytogenes genomic DNA. A fluorogenic probe consisting of a DNA probe labeled with a 5′-dye and a 3′-quencher was included in the same reaction mixture. During PCR amplification, the probe is cleaved, and the reporter dye and quencher are separated. The resulting increase in fluorescence can be detected on the real-time PCR instrument. Two unique and specific primers and probe mixtures were present in this assay. The target for Listeria species is detected in the AFD-Blue dye channel. The target for Listeria monocytogenes is detected in the AFD-Orange dye channel. The target for GFP fluoresce is detected in the AFD-Purple dye channel. An Internal Amplification Control (IAC) is included which is detected in the AFD-Red dye channel. The analysis step is shown in FIG. 1E.

Once the SIMUL-qPCR Assay run was complete, data was analyzed automatically by the software. The software analyzes any DNA amplification data and displays a Cq value for any sample that amplifies. Amplification in the AFD-Blue channel indicates Listeria species. Amplification in the AFD-Orange channel indicates Listeria monocytogenes. Amplification in the AFD-Purple channel indicates GFP. Only a Cq value that had a typical sigmoidal curve or the beginning of the curve was considered positive for the target. When a Cq value is not obtained, the result was determined to be negative for the target provided a Cq value was present in the AFD-Red channel for the IAC. Table 14 shows results interpretation.

TABLE 14
Exemplary Assay Results and Interpretation

| | Results | | | Target Interpretation | | |
|---|---|---|---|---|---|---|
| | Listeria | Listeria | | Listeria | Listeria | |
| Workflow | spp. | monocytogenes | GFP | spp. | monocytogenes | GFP |
| Listeria spp. | No Cq | NDC¹ | No | Negative | | Negative |
| PCR Screen | | | Cq | | | |
| | Cq | NDC¹ | Cq | Positive | | Positive |
| Listeria | NDC¹ | No Cq | No | | Negative | Negative |
| monocytogenes | | | Cq | | | |
| PCR Screen | NDC¹ | Cq | Cq | | Positive | Positive |
| Listeria spp. / | No Cq | No Cq | No | Negative | Negative | Negative |
| Listeria | | | Cq | | | |
| monocytogenes | Cq | No Cq | No | Positive | Negative | Negative |
| PCR Screen | | | Cq | | | |
| | Cq | Cq | No | Positive | Positive | Negative |
| | | | Cq | | | |
| | Cq | Cq | Cq | Positive | Positive | Positive |

Cq = Cq with typical amplification/sigmoidal curve
¹NDC (No data collected), Target not selected during PCR setup, analysis not performed
(Cq in the GFP channel indicates the presence of the GFP quality control strain. Amplification in this channel in any well not containing the GFP quality control strain indicates cross-contamination presence.)

Performance using these methods was found to be comparable to that of the U.S. Department of Agriculture Food Safety and Inspection Service, “Microbiology Laboratory Manual, Isolation and Identification of Listeria monocytogenes from Red Meat, Poultry and Egg Products, and Environmental Samples,” 8.10 (2017), which is hereby incorporated by reference in its entirety, for frankfurters, RTE sliced turkey, and cooked eggs, and the U.S. Food and Drug Administration, “Detection and Enumeration of Listeria monocytogenes in Foods,” Bacteriological Analytical Manual Chapter 10 (2017), which is hereby incorporated by reference in its entirety, for chicken salad, ice cream, pasteurized milk, frozen shrimp, leafy greens, and environmental surface sponges/swabs.

### Results of Matrix Study of Various Food Types

Leafy greens were obtained from a local grocery store and prescreened for Listeria species and L. monocytogenes using the SIMUL-qPCR Listeria species and monocytogenes Assay to determine if any natural contamination was present. The FDA BAM Ch. 10 cultural method was used as the reference method.

To prepare the materials for testing, leafy greens were aseptically combined into a large sterile sampling bag and hand massaged to thoroughly mix the product together. The product was spiked using UV-BioTAG™ L. monocytogenes (1/2a) derived from FDA LS808 sub-cultured in Brain Heart Infusion (BHI) Broth at 37±2° C. for 20-24 h.

The matrix was artificially contaminated at two inoculation levels; a low level to achieve a fractional response of 5-15 positive test portions out of 20 replicate portions tested (approximately 0.2-2 cfu/25 g), and a high level to achieve five positive test portions out of five replicate portions tested (5-10 cfu/25 g). Samples were mixed well to create homogenous mixtures.

The 25 g test portions of leafy greens from each contamination level (including the non-contaminated level) were weighed into a sterile bag for both the candidate and reference methods. The portions were held at 2-8° C. for 48-72 h to allow for stabilization of the microorganisms in the food environment. A 25 g portion from the matrix was also tested for total plate count (6).

For leafy greens, a 25 g portion was added to a sterile container. Two hundred and 25 mL of Buffered Listeria Enrichment Broth (BLEB) containing pyruvate was added to the container and thoroughly mixed by stomaching. The formulation for BLEB with pyruvate was 17 grams of tryptone, 3 grams of soytone, 5 grams of sodium chloride, 2.5 grams of dipotassium phosphate, 2.5 grams of dextrose, 6 grams of yeast extract, 9.6 grams of disodium phosphate, 1.35 grams of monopotassium phosphate, and 1.11 grams of sodium pyruvate. LREB differs from BLEB with pyruvate due to the antibiotics that are included in LREB, which makes it easier to use, saves time and minimizes errors from having to add the antibiotics at a later step. Samples were incubated at 30° C. for 4 h. After the initial incubation, three filter sterilized selective agents were aseptically added to the BLEB to achieve the final concentrations of 10 mg/L acriflavin, 50 mg/L cyclohexamide, and 40 mg/L sodium nalidixic. After mixing, the samples were put back in the 30° C. incubator and incubated for 24-48 h. After 24 h and 48 h, BLEB enrichments were streaked to MOX plates and Rapid L. mono plates. Plates were incubated at 35° C. for MOX agar and 37° C. for Rapid L. mono agar for 48 h. Plates were examined after 24 h. MOX plates were streaked for purity onto TSA YE plates and incubated at 30° C. for 24-48 h. Remaining colony growth was stabbed into 5% sheep blood agar plate and incubated at 35° C. for 24-48 h. Colonies were confirmed using Listeria API strips.

For the SIMUL-qPCR Listeria species and monocytogenes with GFP Assay, each matrix was enriched as described above. After incubation, each matrix was tested as individual portions and in a pooled format. Test portions were lysed, then 5 μL from each test portion lysate was combined with 5 μL from each of the 9 non-inoculated test portion lysates (presumably negative) to create a pool of 10 portions (total volume 50 μL). A pooled sample was prepared to represent each test portion from each level.

An unpaired study was conducted due to the differences in enrichment media and incubations times used between the candidate and reference methods. All samples were processed using either the SIMUL-qPCR Listeria species and monocytogenes with GFP Assay or the FDA BAM Chapter 10 method. All enrichments, including uninoculated samples, were processed for cultural confirmation regardless of the results obtained by the SIMUL-qPCR method. Colonies were confirmed using Listeria API strips.

MPN analyses were conducted on the high and low contamination levels. Test portions from the reference method analysis, plus additional portions from each contamination level were used to create a minimum 5 replicate, 3-level MPN. The FDA BAM Chapter 10 reference method was followed. All MPN results were calculated using the Least Cost Formulations MPN v1.6 calculator program (Virginia Beach, VA).

All test results were analyzed by POD statistical analysis using the AOAC Binary Workbook v5.1 (Least Cost Formulations, Virginia Beach, VA). The paired POD analysis was used to determine presumptive vs confirmed results, and the unpaired analysis was used to compare the candidate method results to the reference method results. The 95% confidence interval on the difference between two POD values must contain 0 for results to be considered not statistically different.

Product Consistency Studies-Three lots of SIMUL-qPCR Listeria Species and monocytogenes with GFP Assay Kit were compared to one lot of the original SIMUL-qPCR Listeria Species and monocytogenes with GFP Assay for lot-to-lot variability.

A pure culture of UV-BioTAG™ L. monocytogenes (1/2a) derived from FDA LS808 was grown in LREB media at 30±1° C. for 30-36 h. The culture was diluted in LREB to a level that yielded fractional results. A pure culture of E. faecalis (NR-31884) was also grown up in BHI at 37° C. for 20-24 h. This culture was not diluted. Each lot of assays were tested with 10 replicates of diluted Listeria tagged with GFP and 10 replicates of undiluted E. faecalis. The samples were blind coded and randomized before being tested.

All test results were analyzed by POD statistical analysis to 95% confidence intervals to determine any variance between lots.

Results of the matrix study are outlined in Tables 15-18. Some challenges were encountered during confirmation testing, and some samples had to be re-streaked to remove some of the competing background flora present in the samples. The background flora, determined by aerobic plate count, was 340 cfu/g for leafy greens. The results in Tables 15-18 include the probability of detection (POD), which is the proportion of positive analytical outcomes for a qualitative method for a given matrix at a given analyte level or concentration. POD is concentration dependent. Several POD measures can be calculated; POD_{R} (reference method POD), POD_{C} (confirmed candidate method POD), POD_{CP} (candidate method presumptive result POD) and POD_{CC} (candidate method confirmation result POD). The difference of probabilities of detection (dPOD) is the difference between any two POD values. If the confidence interval of a dPOD does not contain zero, then the difference is statistically significant at the 5% level.

PCR results were analyzed for the Listeria species target, L. monocytogenes target, as well as the GFP target. For all results listed, all targets called positive to be included in the number of positives displayed. For confirmation of the candidate method, all enriched test portions were processed according to BAM Ch. 10 and confirmed with Listeria API strips. In addition, colonies were viewed under UV light to confirm the presence of the GFP. All samples that confirmed positive for L . . . monocytogenes also confirmed for the presence of GFP.

For the candidate method, 11 out of 20 samples were presumptive for the individual lysates, and 12 out of 20 were presumptive for the pooled lysates. Twelve out of 20 confirmed positive. For the reference method, 9 out of 20 test potions were determined to be positive. No statistically significant differences by POD analysis were seen in any of the comparisons.

Conclusion—These data confirmed that the SIMUL-qPCR Listeria species and monocytogenes with GFP Assay was suitable for detecting Listeria species and monocytogenes in leafy greens. Furthermore, the data confirmed that the method was valid for pooled lysates. POD analysis showed that there were no statistically significant differences between the candidate method and the FDA BAM Chapter 10 reference method in the matrix study. The study also demonstrated that the assay could yield consistent results compared to the old assay that did not contain the GFP target. Finally, the study proved that the assay could detect a GFP target if a Listeria strain is tagged with the GFP, which can add value to laboratories in limiting time and labor in investigations into possible internal cross-contamination issues.

TABLE 15
SIMUL-qPCR Listeria species and monocytogenes with GFP Assay
Presumptive vs. Confirmed Results -Individual lysates

| | | | | SIMUL-qPCR Listeria | | | SIMUL-qPCR Listeria | | | | |
|---|---|---|---|---|---|---|---|---|---|---|---|
| | | | | species and monocytogenes | | | species and monocytogenes | | | | |
| | | | | with GFP Presumptive | | | with GFP Confirmed | | | | |
| Matrix | Strain | MPN^{a}/25 g | N^{b} | x^{c} | POD_{CP}^{d} | 95% CI | x | POD_{CC}^{e} | 95% CI | dPOD_{CP}^{f} | 95% CI^{g} |
| Leafy | Listeria | N/A | 5 | 0 | 0.00 | 0.00, 0.43 | 0 | 0.00 | 0.00, 0.43 | 0.00 | −0.43, |
| Greens | monocytogenes | | | | | | | | | | 0.43 |
| 30 h | (½a)^{h} | 0.77 (0.46, | 20 | 11 | 0.55 | 0.34, 0.74 | 12 | 0.60 | 0.39, 0.78 | −0.05 | −0.33, |
| | | 1.25) | | | | | | | | | 0.24 |
| | | 1E+12 (5.07, | 5 | 5 | 1.00 | 0.57, 1.00 | 5 | 1.00 | 0.57, 1.00 | 0.00 | −0.43, |
| | | 1E+12)ⁱ | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values
^{g}95% CI = If the confidence interval of a dPOD does not contain zero, then the difference is statistically significant at the 5% level.
^{h}UV-BioTag ™ Listeria [L. monocytogenes (½a) derived from FDA LS808]; Microbiologics, Inc., St. Cloud, Minnesota.
ⁱAll test portions in the MPN were positive, leading to the high value.

TABLE 16
SIMUL-qPCR Listeria species and monocytogenes with GFP
Assay vs. Reference Method Results - Individual lysates

| | | | | SIMUL-qPCR Listeria | | | | | | | |
|---|---|---|---|---|---|---|---|---|---|---|---|
| | | | | species and monocytogenes | | | | | | | |
| | | | | with GFP Results | | | BAM Ch. 5 | | | | |
| Matrix | Strain | MPN^{a}/25 g | N^{b} | x^{c} | POD_{C}^{d} | 95% CI | x | POD_{R}^{e} | 95% CI | dPOD_{C}^{f} | 95% CI^{g} |
| Leafy | Listeria | N/A | 5 | 0 | 0.00 | 0.00, 0.43 | 0 | 0.00 | 0.00, 0.43 | 0.00 | −0.43, |
| Greens | monocytogenes | | | | | | | | | | 0.43 |
| 30 h | (½a)^{h} | 0.77 (0.46, | 20 | 11 | 0.55 | 0.34, 0.74 | 9 | 0.45 | 0.26, 0.66 | 0.10 | −0.19, |
| | | 1.25) | | | | | | | | | 0.37 |
| | | 1E+12 (5.07, | 5 | 5 | 1.00 | 0.57, 1.00 | 5 | 1.00 | 0.57, 1.00 | 0.00 | −0.43, |
| | | 1E+12)ⁱ | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method final result and reference method result POD values
^{g}95% CI = If the confidence interval of a dPOD does not contain zero, then the difference is statistically significant at the 5% level.
^{h}UV-BioTag ™ Listeria [L. monocytogenes (½a) derived from FDA LS808]; Microbiologics, Inc., St. Cloud, Minnesota.
ⁱAll test portions in the MPN were positive, leading to the high value.

TABLE 17
SIMUL-qPCR Listeria species and monocytogenes with GFP
Assay Presumptive vs. Confirmed Results - Pooled lysates

| | | | | SIMUL-qPCR Listeria | | | SIMUL-qPCR Listeria | | | | |
|---|---|---|---|---|---|---|---|---|---|---|---|
| | | | | species and monocytogenes | | | species and monocytogenes | | | | |
| | | | | with GFP Presumptive | | | with GFP Confirmed | | | | |
| Matrix | Strain | MPN^{a}/25 g | N^{b} | x^{c} | POD_{CP}^{d} | 95% CI | x | POD_{CC}^{e} | 95% CI | dPOD_{CP}^{f} | 95% CI^{g} |
| Leafy | Listeria | N/A | 5 | 0 | 0.00 | 0.00, 0.43 | 0 | 0.00 | 0.00, 0.43 | 0.00 | −0.43, |
| Greens | monocytogenes | | | | | | | | | | 0.43 |
| 30 h | (½a)^{h} | 0.77 (0.46, | 20 | 12 | 0.60 | 0.39, 0.78 | 12 | 0.60 | 0.39, 0.78 | 0.00 | −0.28, |
| | | 1.25) | | | | | | | | | 0.28 |
| | | 1E+12 (5.07, | 5 | 5 | 1.00 | 0.57, 1.00 | 5 | 1.00 | 0.57, 1.00 | 0.00 | −0.43, |
| | | 1E+12)ⁱ | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values
^{g}95% CI = If the confidence interval of a dPOD does not contain zero, then the difference is statistically significant at the 5% level.
^{h}UV-BioTag ™ Listeria [L. monocytogenes (½a) derived from FDA LS808]; Microbiologics, Inc., St. Cloud, Minnesota.
ⁱAll test portions in the MPN were positive, leading to the high value.

TABLE 18
SIMUL-qPCR Listeria species and monocytogenes with GFP Assay vs. Reference Method Results - Pooled lysates

| | | | | SIMUL-qPCR Listeria | | | | | | | |
|---|---|---|---|---|---|---|---|---|---|---|---|
| | | | | species and monocytogenes | | | FDA BAM | | | | |
| | | | | with GFP Results | | | Ch. 10 Results | | | | |
| Matrix | Strain | MPN^{a}/25 g | N^{b} | x^{c} | POD_{C}^{d} | 95% CI | x | POD_{R}^{e} | 95% CI | dPOD_{C}^{f} | 95% CI^{g} |
| Leafy | Listeria | N/A | 5 | 0 | 0.00 | 0.00, 0.43 | 0 | 0.00 | 0.00, 0.43 | 0.00 | −0.43, |
| Greens | monocytogenes | | | | | | | | | | 0.43 |
| 30 h | (½a)h | 0.77 (0.46, | 20 | 12 | 0.60 | 0.39, 0.78 | 9 | 0.45 | 0.26, 0.66 | 0.15 | −0.15, |
| | | 1.25) | | | | | | | | | 0.41 |
| | | 1E+12 (5.07, | 5 | 5 | 1.00 | 0.57, 1.00 | 5 | 1.00 | 0.57, 1.00 | 0.00 | −0.43, |
| | | 1E+12)ⁱ | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method final result and reference method result POD values
^{g}95% CI = If the confidence interval of a dPOD does not contain zero, then the difference is statistically significant at the 5% level.
^{h}UV-BioTag ™ Listeria [L. monocytogenes (½a) derived from FDA LS808]; Microbiologics, Inc., St. Cloud, Minnesota.
ⁱAll test portions in the MPN were positive, leading to the high value.

### Example 3—Top 7 STEC with GFP Assay

A quick and reliable method for detecting the Top 7 Shiga toxin-producing E. coli in raw beef trim, raw ground beef, leafy greens, dried hemp flower and spunbonded polyolefin sampling sheets was developed. A workflow of this protocol is illustrated in FIGS. 1A-E. Each assay utilizes the power of multiplexing several targets during the same run. The SIMUL-qPCR Top 7 STEC Assay incorporated a multiplex approach to identify E. coli O157:H7, O26, O45, O103, O111, O121, O145, Shiga toxin and intimin genes. The SIMUL-qPCR E. coli O157:H7 with GFP Assay incorporated a real-time PCR approach to identifying E. coli O157:H7 and an added layer of assurance with the addition of the Green Fluorescent Protein (GFP) positive control. The test sample and control sample (strain engineered with GFP) are shown in FIG. 1A.

The test kit evaluated for the detection of Escherichia coli, included the reported labeled strain, UVQC E. coli (O157:H7), which contains a Green Fluorescent Protein (GFP) marker. E. coli O157:H7 does not naturally contain GFP. The Green Fluorescent Protein (GFP) marker amplifies with the E. coli in the SIMUL-qPCR E. coli with GFP Assay, allowing easy, immediate differentiation upon detection of the target pathogen. Subsequently, UVQC E. coli produced visible fluorescent colonies on selective agar plates under UV light after isolation from the enrichment media. By utilizing an E. coli O157:H7 quality control strain tagged with GFP, it could be determined if any potential positives were caused by cross-contamination. Previously, potential positives would need to be streaked for cultural confirmation and then the colonies would need to be examined under a UV light with the appropriate wavelength, adding days to the process of determining whether any cross-contamination had occurred. With this assay, it could be determined by the end of the PCR run.

The Simultaneous Multiplex Real Time PCR (SIMUL-qPCR) Top 7 STEC Assay has been validated by the AOAC™ Research Institute under the Performance Test Methods™ program for raw beef trim, raw ground beef, leafy greens, and sampling sheets. The USDA FSIS MLG method was used for method comparison testing. The SIMUL-qPCR Top 7 STEC Assay was found to be equivalent to the reference method. The limit of detection for this SIMUL-qPCR assay was determined to be 10,000 CFU/mL of enriched sample.

TABLE 19
Exemplary Multiplex Combinations

| KIT NAME | AFD-Blue | AFD-Orange | AFD-Red |
|---|---|---|---|
| SIMUL-qPCR E. coli | E. coli | GFP | IAC |
| O157:H7 with GFP Kit | O157:H7 | | |
| SIMUL-qPCR Shiga Toxin | Intimin (eae) | Shiga toxin | IAC |
| (stx) and Intimin (eae) Gene | gene | (stx) gene | |
| STEC Kit | | | |
| SIMUL-qPCR E. coli O26 | E. coli O103 | E. coli O26 | IAC |
| and E. coli O103 STEC Kit | | | |
| SIMUL-qPCR E. coli | E. coli O145 | E. coli O111 | IAC |
| O111 and E. coli | | | |
| O145 STEC Kit | | | |
| SIMUL-qPCR E. coli O45 | E. coli O45 | E. coli O121 | IAC |
| and E. coli O121 STEC Kit | | | |

### Selective Enrichment Media & Preparation

Enterohemorrhagic E. coli Recovery and Enrichment Broth (EREB) is a selective medium, specifically optimized for single-step recovery and enrichment of Enterohemorrhagic E. coli (EHEC) from raw beef trim, ground beef, leafy greens, and sampling sheets. The medium can also be used for the recovery and enrichment of Salmonella as a single-enrichment method alongside EHEC in beef samples, and leafy greens. Buffered Peptone Water can be used for the recovery and enrichment of Salmonella and EHEC in dried hemp flower. The enrichment step is shown in FIG. 1B.

37.8 g (EREB) or 20 g (BPW) of powder was combined with 1 L of distilled water in a sterile bottle, and stirred and heated until dissolved. The acceptable pH was 7.2±0.2. The media can be autoclaved or cooled to the appropriate temperature (45±1° C.) and used immediately.

### Sample Preparation

Food Sample: In general, food products were aseptically sampled or the rinse poured into a sterile bag. When ready to be tested, the prepared EREB medium was pre-warmed to 45±1° C. and added to the sample. Enrichment of Samples Tables 20 and 21 have acceptable media volumes for various food types. Samples were hand mixed by massaging each sample that is in the sealed bag for approximately one minute to homogenize, and then incubated. The Enrichment of Samples Tables 20-21 show enrichment incubation conditions for various food types.

For example, raw beef trim and raw ground beef samples were prepared for testing by aseptically sampling the product and placing it in a sterile bag. 1 L+50 mL of pre-warmed EREB was added to the sample and the bag was sealed. Each sample was hand mixed by massaging in the bag for approximately one minute to homogenize. The sample was incubated at 42° C.±1° C. for 10-18 h. If the samples were to be pooled, the incubation time was increased to 12-18 h.

To prepare leafy green samples, 200 grams was aseptically weighed out into a sterile filtered stomacher bag and 450 mL±30 mL of pre-warmed EREB was added to the sample.

Dried Hemp Flower: For dried hemp flower, 10 g was aseptically sampled and placed into a sterile filtered stomacher bag. 90 mL±10 mL of pre-warmed (37° C.) BPW was added to the sample and the bag sealed. Each sample was hand mixed by massaging in the bag for approximately one minute to homogenize. The samples were incubated at 42±1° C. for 24 h.

Enrichment of Samples Tables 20 (single samples) or Table 21 (pooled samples) contain additional example Enrichment conditions. Table 20 Enrichment Incubation: 42±1° C. for 10-18 hours, unless otherwise indicated. Table 21 Enrichment Incubation: 42±1° C. for 12-18 hours, unless otherwise indicated.

Sampling Sheet: When ready to test, the prepared EREB was pre-warmed to 45±1° C. and added to the sample. Enrichment of Samples Tables 20 and 21 have acceptable media volumes for various sample types. Each sample was hand mixed by massaging in the bag for approximately one minute to homogenize and incubated. Enrichment of Samples Tables 20-21 show enrichment conditions. For example, 200 mL of pre-warmed EREB was added to a bag with sampling sheets and hand mixed by massaging each sample in the bag before closing the bag. The sample was incubated at 42° C.±1° C. for 10-18 h. If the samples were to be pooled, the incubation time was increased to 12-18 h.

TABLE 20
Enrichment of Single Samples

| | SAMPLE SIZE/ | | MEDIA |
|---|---|---|---|
| MATRIX | ANALYSIS UNIT | | VOLUME |
| Raw Ground Beef | /375 | g | 1 L ± 50 mL |
| Raw Beef Excision Sample N60 | /375 | g | 1 L ± 50 mL |
| Spunbonded Polyolefin | /sheet | | 200 mL ± 15 mL |
| Sampling Sheet | | | |
| Leafy Greens | /200 | g | 450 mL ± 30 mL |
| Dried Hemp Flower* | /10 | g | 90 mL ± 10 mL |

*Dried hemp flower incubates for 24 hours in BPW.

TABLE 21
Enrichment of Single Samples

| | SAMPLE SIZE/ | | MEDIA |
|---|---|---|---|
| MATRIX | ANALYSIS UNIT | | VOLUME |
| Raw Ground Beef | /375 | g | 1 L ± 50 mL |
| Raw Beef Excision Sample N60 | /375 | g | 1 L ± 50 mL |
| Spunbonded Polyolefin | /sheet | | 200 mL ± 15 mL |
| Sampling Sheet | | | |
| Leafy Greens | /200 | g | 450 mL ± 30 mL |
| Dried Hemp Flower | /10 | g | 90 mL ± 10 mL |

*Dried hemp flower incubates for 24 hours in BPW.

### Cell Lysis Protocol

After incubation, one 1.5 mL microcentrifuge tube or equivalent PCR-grade plastic tube was labeled per sample. 400 μL of lysis buffer was pipetted into each labeled tube. For sampling sheets and hemp flower, 995 μL of lysis buffer was pipetted into each labeled tube. 5 μL of the enrichment broth was pipetted into the prepared tube. The tube(s) were capped and vortexed. The closed tubes were heated for 10 minutes at 95±3° C. in the heat block, then cooled for 5 minutes at room temperature. The samples were used directly in the SIMUL-qPCR assay or the lysate was held in a refrigerator (2-8° C.) up to 48 hours before proceeding to SIMUL-qPCR assay. The lysis step is shown in FIG. 1C.

For samples that were enriched for the minimum time to support pooling: up to 10 samples were pooled immediately after cell lysis. For pooling, 5 μL aliquots from each lysate were combined in a microcentrifuge tube or equivalent PCR-grade plastic tube (total volume should not exceed 50 μL). The pooled samples were pipetted to mix the samples and used directly in the SIMUL-qPCR assay. The qPCR set up and data entry was set up as described in Example 1 and is shown in FIG. 1D.

### Interpreting Results

During PCR amplification, forward and reverse primers hybridize to unique sequences of the target's genomic DNA. A fluorogenic probe is included in the same reaction mixture which consists of a DNA probe labeled with a 5′-dye and a 3′-quencher. During PCR amplification, the probe is cleaved, and the reporter dye and quencher are separated. The resulting increase in fluorescence can be detected on the real-time PCR instrument. The collection is composed of 5 unique PCR reaction tubes with multiple STEC targets which are to be run simultaneously but may be processed individually. Table 22 specifies the targets and corresponding fluorescence channels.

Once the run was complete, data was analyzed automatically by the software. The software analyzes any DNA amplification data and will display a Cq value for any sample that amplifies. Only a Cq value that has a typical sigmoidal curve or the beginning of the curve is considered positive for the target. When a Cq value is not obtained, the result is negative for the target provided a Cq value is present in the AFD-Red channel for the IAC. The analysis step is shown in FIG. 1E.

All positive results were considered potential positives and confirmed by plating on selective agar plates, typically rainbow agar (manufactured by Biolog, Inc.) and Macconkey agar with sorbitol (formula per liter is 20 grams of peptone, 10 grams of D-sorbitol, 1.5 grams of bile salts, 5 grams of sodium chloride, 0.03 grams of neutral red, 0.001 grams of crystal violet, and 15 grams of agar. Enriched samples were confirmed using either the FSIS MLG method (“Microbiology Laboratory Guidebook, 5.09, Detection, Isolation and Identification of Escherichia coli O157:H7 from Meat Products and Carcass and Environmental Sponges,” U.S. Department of Agriculture-Food Safety and Inspection Service (2015), which is hereby incorporated by reference in its entirety). or the FDA BAM method (Feng, et al., “Chapter 4A: Diarrheagenic Escherichia coli.” U.S. Food and Drug Administration Bacteriological Analytical Manual (BAM), (2020), which is hereby incorporated by reference in its entirety) using the enrichment broth, stored at 2-8° C. Some positive results were difficult to culturally confirm due to low levels of E. coli target cells, high levels of background flora, or a combination of these factors.

TABLE 22
Exemplary Assay Results and Interpretation

| | Fluorescence | Target |
|---|---|---|
| Workflow | Channel | Interpretation |
| SIMUL-qPCR E. coli | AFD Blue | E. coli O157:H7 |
| O157:H7 with GFP Kit | AFD Orange | GFP*** |
| SIMUL-qPCR Shiga Toxin | AFD Orange | Shiga Toxin (stx) |
| (stx) and Intimin (eae) Gene | | |
| STEC Kit | AFD Blue | Intimin (eae) |
| SIMUL-qPCR E. coli O26 | AFD Orange | E. coli O26 |
| and E. coli O103 STEC Kit | AFD Blue | E. coli O103 |
| SIMUL-qPCR E. coli | AFD Orange | E. coli O111 |
| O111 and E. coli | AFD Blue | E. coli O145 |
| O145 STEC Kit | | |
| | AFD Blue | E. coli O45 |
| SIMUL-qPCR E. coli O45 | AFD Orange | E. coli O121 |
| and E. coli O121 STEC Kit | | |

***Cq in the GFP channel (AFD Orange) indicates the presence of the GFP quality control strain. Amplification in this channel in any well not containing the GFP quality control strain indicates cross-contamination presence.

### Results of Matrix Study of Various Food Types

Leafy greens were obtained from a local grocery store. Dried hemp flower was obtained from a hemp producer in Oregon. The matrixes were prescreened for the Top 7 STEC strains and for Salmonella using the SIMUL-qPCR Top 7 STEC and Salmonella assays prior to testing to determine if any natural contamination was present. Note: Because the EREB and BPW media are designed to enrich for both STEC and Salmonella, the matrixes were co-inoculated with an STEC strain and a Salmonella strain and then tested by the SIMUL-qPCR E. coli O157:H7 with GFP and SIMUL-qPCR Salmonella Assays. The results of the Salmonella testing by the SIMUL-qPCR Salmonella Assay are presented in Example 4.

To prepare the materials for testing, each product for each matrix was aseptically combined into a large sterile sampling bag and hand massaged to thoroughly mix the product together. The product was spiked using strains sub-cultured in BHI at 37±2° C. for 20-24 h. The matrixes were inoculated with E. coli O157:H7 EC43 derived from FDA ESC1177 (STEC) as that strain includes the GFP target. Both matrixes were co-inoculated with S. Typhimurium Sal54 derived from SAL5694.

Each matrix was artificially contaminated with the indicated strains at two contamination levels; a low level to achieve a fractional response of 5-15 positive test portions out of 20 replicate portions tested (approximately 0.2-2 cfu/25 g), and a high level to achieve five positive test portions out of five replicate portions tested (approximately 5-10 cfu/25 g). Samples were mixed well to homogenize mixtures.

For leafy greens, 25 g portions of each contamination level (including the uncontaminated level) were weighed into a sterile sampling bag. For both the candidate and reference methods, an additional 175 g of uninoculated product was added to bring the weight of each portion to 200 g. Note: The reference method test portion for Salmonella testing is 25 g and is discussed in Example 4. The samples were held at 2-8° C. for 48-72 h to allow for stabilization of the microorganisms in the food environment. For dried hemp flower, 10 g test portions of each contamination level (including the uncontaminated level) were weighed into a sterile filtered stomacher bag and held at room temperature (20-25° C.) for 2 weeks.

For the candidate method, leafy greens were tested at 10 and 18 h for single portions and 12 and 18 h for pooled portions, and dried hemp flower was tested at 24 h for both individual and pooled lysates. The reference method for leafy greens was tested at 24 h. Each matrix was also tested for aerobic plate count (see See Maturin and Peeler, “BAM Chapter 3: Aerobic Plate Count,” U.S. Food and Drug Administration Bacteriological Analytical Manual, which is hereby incorporated by reference in its entirety).

For leafy greens, the BAM method was used for the reference method. For dried hemp flower, SMPR 2020.0012 Detection of Shiga Toxin-Producing Escherichia coli in Cannabis and Cannabis Products was followed. Test portions were enriched in 450 mL of modified Buffered Peptone Water with Pyruvate and incubated for 5 h at 37±1° C. before adding 1 mL each of acriflavine, cefsulodin, and vancomycin to the enrichment. Once the supplements were added, the portions were then incubated at 42±1° C. for 18-24 h. The samples were then streaked onto Rainbow® agar and Tellurite Cefixime-Sorbitol MacConkey Agar (TC-SMAC) following the BAM method to ensure the products were not naturally contaminated. For dried hemp flower, the enrichment outlined by the SIMUL-qPCR method was used.

An unpaired study was conducted on leafy greens due to the difference in enrichment media and incubation times used between the candidate and reference methods. Test portions were either processed using the SIMUL-qPCR Top 7 STEC Assay or the BAM, depending on the enrichment. For dried hemp flower, test portions were processed using the SIMUL-qPCR Top 7 STEC Assay only, since there was no reference method enrichment. All enriched portions were processed for cultural confirmation per the BAM procedure regardless of the results obtained by the SIMUL-qPCR method. Enrichments were streaked to TC-SMAC and Rainbow agar plates. Colonies were then taken to Trypticase Soy Agar with Yeast Extract plates for purity. Latex agglutination for both the 0157 antigen and H7 antigen were performed. Any latex-positive colonies were processed for biochemical identification using the VITEK method (bioMerieux, Hazelwood, MO). Both TC-SMAC plates and Rainbow agar plates were also looked at under a 400 nm UV light to confirm the presence of the Green Fluorescent Protein in the colonies.

MPN analyses were conducted on the high and low contamination levels. Test portions from the reference method analysis (BAM), plus additional portions from each contamination level were used to create a minimum 5 replicate, 3-level MPN. All MPN results were calculated using the MPN v1.6 calculator program (Least Cost Formulations, Virginia Beach, VA).

All test results were analyzed by POD statistical analysis using the AOAC Binary Workbook v5.1 (Least Cost Formulations, Virginia Beach, VA), which is the proportion of positive analytical outcomes for a qualitative method for a given matrix at a given analyte level or concentration. POD is concentration dependent. Several POD measures can be calculated; POD_{R} (reference method POD), POD_{C} (confirmed candidate method POD), POD_{CP} (candidate method presumptive result POD) and POD_{CC} (candidate method confirmation result POD). The difference of probabilities of detection (dPOD) is the difference between any two POD values. The paired POD analysis was used to determine presumptive vs confirmed results, and the unpaired analysis was used to compare the candidate method results to the reference method results. The 95% confidence interval on the difference between two POD values must contain 0 for results to be considered not statistically different.

Results of the matrix study are outlined in Tables 23-26. Based on previous matrix studies, it was determined that incubation time should be two hours longer when pooling lysates from individual test portions than the incubation time for single lysate testing (12 h vs. 10 h). During confirmation testing, some samples had to be re-streaked to remove some of the competing background flora present in the samples. The background flora for each matrix, determined by aerobic plate count, was 340 cfu/g for leafy greens, and 20 cfu/g for hemp.

PCR results were analyzed for both the E. coli O157:H7 target as well as the GFP target. For all results listed, both targets called positive to be included in the number of positives displayed. For confirmation of the candidate method, colonies were viewed under UV light to confirm the presence of the GFP. All samples that confirmed positive for E. coli O157:H7 also confirmed for the presence of GFP.

For the leafy green test portions, 13 out of 20 portions were presumptive positive for the candidate method for single lysates at both the 10 and 18 h time points. For the pooled lysates, 13 out of 20 portions were presumptive positive at the 12 h time point, and 15 portions were presumptive positive at the 18 h time point. Thirteen out of 20 test portions confirmed positive. For the reference method, 14 out of 20 test portions confirmed positive, indicating no statistically significant difference between the candidate and reference methods results at either time point. Also, the candidate method showed no significant difference by POD analysis between individual and pooled results.

For dried hemp flower, 13 out of 20 test portions were presumptive positive for the candidate method for single lysates after 24 h. For the pooled lysates, 12 out of 20 test portions were presumptive positive. Thirteen out of 20 portions confirmed positive. There were no statistically significant differences between the presumptive and confirmed results for individual and pooled portions, or between incubation time points.

Conclusion—These data confirmed that the SIMUL-qPCR E. coli O157:H7 with GFP Assay, part of the Top 7 STEC Assay Collection, was suitable for detecting Top 7 STEC species in leafy greens and dried hemp flower. Furthermore, the data confirmed that the method was valid for pooled lysates. POD analysis showed that there were no statistically significant differences between the candidate method results and the BAM Chapter 4 reference method results for any of the for leafy greens, or between presumptive and confirmed results for dried hemp flower (≤0.3% THC). The study also demonstrated that the assay could yield consistent results over multiple different lots. Finally, the study proved that the assay could detect a GFP target if a E. coli O157:H7 strain is tagged with the GFP, which can add value to laboratories in limiting time and labor in investigations into possible internal cross-contamination issues.

TABLE 23
SIMUL-qPCR E. coli O157:H7 with GFP Assay Presumptive vs. Confirmed
Results - Individual lysates for leafy greens and dried hemp flower

| | | | | SIMUL-qPCR E. coli | | | SIMUL-qPCR E. coli | | | | |
|---|---|---|---|---|---|---|---|---|---|---|---|
| | | | | O157:H7 with GFP | | | O157:H7 with GFP | | | | |
| | | | | Presumptive | | | Confirmed | | | | |
| Matrix | Strain | MPN^{a}/200 g | N^{b} | x^{c} | POD_{CP}^{d} | 95% CI | x | POD_{CC}^{e} | 95% CI | dPOD_{CP}^{f} | 95% CI^{g} |
| Leafy | E. coli | N/Aⁱ | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 10 h | EC 43 | 1.097 | 20 | 13 | 0.65 | 0.43, 0.82 | 13 | 0.65 | 0.43, 0.82 | 0.00 | −0.28, |
| | derived | (0.69, 1.86) | | | | | | | | | 0.28 |
| | from FDA^{h} | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Leafy | E. coli | N/A | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 18 h | EC 43 | 1.097 | 20 | 13 | 0.65 | 0.43, 0.82 | 13 | 0.65 | 0.43, 0.82 | 0.00 | −0.28, |
| | derived | (0.69, 1.86) | | | | | | | | | 0.28 |
| | from FDA | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Dried | E. coli | N/A | 5 | 0 | 0.00 | 0.00, 0.43 | 0 | 0.00 | 0.00, 0.43 | 0.00 | −0.43, |
| Hemp | O157:H7 | | | | | | | | | | 0.43 |
| Flower | EC 43 | 1.33 | 20 | 13 | 0.65 | 0.43, 0.82 | 13 | 0.65 | 0.43, 0.82 | 0.00 | −0.28, |
| 24 h | derived | (0.84, 2.18) | | | | | | | | | 0.28 |
| | from FDA | 1E+12 | 5 | 5 | 1.00 | 0.57, 1.00 | 5 | 1.00 | 0.57, 1.00 | 0.00 | −0.43, |
| | ESC1177 | (8.18, 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values.
^{g}95% CI = If the confidence interval of a dPOD does not contain zero, then the difference is statistically significant at the 5% level.
^{h}FDA = Food and Drug Administration, Silver Spring, MD;
ⁱN/A = Not applicable.

TABLE 24
SIMUL-qPCR E. coli O157:H7 with GFP Assay vs. Reference
Method Results - Individual lysates for leafy greens

| | | | | SIMUL-qPCR E. coli | | | | | | | |
|---|---|---|---|---|---|---|---|---|---|---|---|
| | | | | O157:H7 with GFP | | | BAM Ch. 4A | | | | |
| Matrix | Strain | MPN^{a}/200 g | N^{b} | x^{c} | POD_{C}^{d} | 95% CI | x | POD_{R}^{e} | 95% CI | dPOD_{C}^{f} | 95% CI^{g} |
| Leafy | E. coli | N/Aⁱ | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 10 h | EC 43 | 1.097 | 20 | 13 | 0.65 | 0.43, 0.82 | 14 | 0.70 | 0.48, 0.85 | −0.05 | −0.32, |
| | derived | (0.69, 1.86) | | | | | | | | | 0.23 |
| | from FDA^{h} | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Leafy | E. coli | N/A | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 18 h | EC 43 | 1.097 | 20 | 13 | 0.65 | 0.43, 0.82 | 14 | 0.70 | 0.48, 0.85 | −0.05 | −0.32, |
| | derived | (0.69, 1.86) | | | | | | | | | 0.23 |
| | from FDA | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}FDA = Food and Drug Administration, Silver Spring, MD.
ⁱN/A = Not applicable.

TABLE 25
SIMUL-qPCR E. coli O157:H7 with GFP Assay Presumptive vs. Confirmed
Results - Pooled lysates for leafy greens and dried hemp flower

| | | | | SIMUL-qPCR E. coli | | | SIMUL-qPCR E. coli | | | | |
|---|---|---|---|---|---|---|---|---|---|---|---|
| | | | | O157:H7 with GFP | | | O157:H7 with GFP | | | | |
| | | | | Presumptive | | | Confirmed | | | | |
| Matrix | Strain | MPN^{a}/200 g | N^{b} | x^{c} | POD_{CP}^{d} | 95% CI | x | POD_{CC}^{e} | 95% CI | dPOD_{CP}^{f} | 95% CI^{g} |
| Leafy | E. coli | N/Aⁱ | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 12 h | EC 43 | 1.097 | 20 | 13 | 0.65 | 0.43, 0.82 | 13 | 0.65 | 0.43, 0.82 | 0.00 | −0.28, |
| | derived | (0.69, 1.86) | | | | | | | | | 0.28 |
| | from FDA^{h} | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Leafy | E. coli | N/A | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 18 h | EC 43 | 1.097 | 20 | 15 | 0.75 | 0.53, 0.89 | 13 | 0.65 | 0.43, 0.82 | 0.10 | −0.18, |
| | derived | (0.69, 1.86) | | | | | | | | | 0.36 |
| | from FDA | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Dried | E. coli | N/A | 5 | 0 | 0.00 | 0.00, 0.43 | 0 | 0.00 | 0.00, 0.43 | 0.00 | −0.43, |
| Hemp | O157:H7 | | | | | | | | | | 0.43 |
| Flower | EC 43 | 1.33 | 20 | 12 | 0.60 | 0.39, 0.78 | 13 | 0.65 | 0.43, 0.82 | −0.05 | −0.32, |
| 24 h | derived | (0.84, 2.18) | | | | | | | | | 0.23 |
| | from FDA | 1E+12 | 5 | 5 | 1.00 | 0.57, 1.00 | 5 | 1.00 | 0.57, 1.00 | 0.00 | −0.43, |
| | ESC1177 | (8.18, 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}POD_{CC} = Candidate method confirmed positive outcomes divided by the total number of trials.
^{f}dPOD_{CP} = Difference between the candidate method presumptive result and candidate method confirmed result POD values.
^{g}95% CI = If the confidence interval of a dPOD does not contain zero, then the difference is statistically significant at the 5% level.
^{h}FDA = Food and Drug Administration, Silver Spring, MD;
ⁱN/A = Not applicable.

TABLE 26
SIMUL-qPCR E. coli O157:H7 with GFP Assay vs. Reference Method Results - Pooled lysates for leafy greens

| | | | | SIMUL-qPCR E. coli | | | | | | | |
|---|---|---|---|---|---|---|---|---|---|---|---|
| | | | | O157:H7 with GFP | | | | | | | |
| | | | | Confirmed | | | BAM Ch. 4A | | | | |
| Matrix | Strain | MPN^{a}/200 g | N^{b} | x^{c} | POD_{C}^{d} | 95% CI | x | POD_{R}^{e} | 95% CI | dPOD_{C}^{f} | 95% CI^{g} |
| Leafy | E. coli | N/Aⁱ | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 12 h | EC 43 | 1.097 | 20 | 13 | 0.65 | 0.43, 0.82 | 14 | 0.70 | 0.48, 0.85 | −0.05 | −0.32, |
| | derived | (0.69, 1.86) | | | | | | | | | 0.23 |
| | from FDA^{h} | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |
| Leafy | E. coli | N/A | 5 | 0 | 0.00 | 0.00, 0.43 | 0 | 0.00 | 0.00, 0.43 | 0.00 | −0.43, |
| Greens | O157:H7 | | | | | | | | | | 0.43 |
| 18 h | EC 43 | 1.097 | 20 | 13 | 0.65 | 0.43, 0.82 | 14 | 0.70 | 0.48, 0.85 | −0.05 | −0.32, |
| | derived | (0.69, 1.86) | | | | | | | | | 0.23 |
| | from FDA | 1E+12 | 5 | 5 | 1.00 | 0.57, 1.00 | 5 | 1.00 | 0.57, 1.00 | 0.00 | −0.43, |
| | ESC1177 | (4.73, 1E+12) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}DA = Food and Drug Administration, Silver Spring, MD.
ⁱN/A = Not applicable.

### Example 4—Salmonella and Top 7 STEC with GFP Assay

This protocol was tested as a multifaceted approach to the detection of Salmonella species as well as STEC in leafy greens, dried Cannabis flower, and dried hemp flower. A workflow of this protocol is illustrated in FIGS. 1A-E. Specifically formulated media were utilized for enriching samples followed by lysis of samples, and rapid (qPCR) detection procedures as described in Examples 1-3.

Buffered Peptone Water (BPW) contains necessary nutritional components for the growth of Salmonella. AFD E. coli Recovery and Enrichment Broth (EREB) combines those nutritional components incorporated in BPW with additional ingredients that are necessary to selectively improve recovery and growth of Salmonella. The selective agents present in EREB were optimized to efficiently inhibit competing bacterial flora without affecting the growth of Salmonella species. BPW and EREB were formulated for buffering capacity to ensure growth in a variety of matrices. It was determined that EREB should be used with leafy greens and BPW should be used with dried hemp and Cannabis flower.

### Interpreting Results

The qPCR software analyzes any DNA amplification data and potential results are indicated with a Cq result. However, review is necessary to determine if the Cq has sigmoidal or the beginning of sigmoidal amplification, corresponding to a true potential positive. When a Cq value is not obtained, the result is negative for the target provided a Cq value is present in the AFD-Red channel for the IAC. The GFP target is located in the AFD-Purple channel. If a control strain labeled with the GFP was used during a PCR run, any samples that have a Cq value present in the AFD-Purple channel represents the presence of the GFP target and a potential cross-contamination between the control strain and the sample.

TABLE 27
Exemplary Assay Results and Interpretation
SIMUL-qPCR Salmonella & STEC with GFP Assay

| | | | | (AFD-Red) | |
|---|---|---|---|---|---|
| | | | | Internal | |
| | AFD- | AFD- | AFD- | Ampli- | |
| | Blue | Orange | Purple | fication | Reportable |
| | (Salmonella) | (STEC) | (GFP) | Control | Result |
| Result | C_{q} | C_{q} | C_{q} | C_{q} | Pos: Sal |
| | | | | | Pos: STEC |
| | | | | | Pos: GFP |
| Result | C_{q} | C_{q} | No C_{q} | C_{q} | Pos: Sal |
| | | | | | Pos: STEC |
| | | | | | Neg: GFP |
| Result | C_{q} | No C_{q} | C_{q} | C_{q} | Pos: Sal |
| | | | | | Neg: STEC |
| | | | | | Pos: GFP |
| Result | No C_{q} | C_{q} | C_{q} | C_{q} | Neg: Sal |
| | | | | | Pos: STEC |
| | | | | | Pos: GFP |
| Result | C_{q} | No C_{q} | No C_{q} | C_{q} | Pos: Sal |
| | | | | | Neg: STEC |
| | | | | | Neg: GFP |
| Result | No C_{q} | C_{q} | No C_{q} | C_{q} | Neg: Sal |
| | | | | | Pos: STEC |
| | | | | | Neg: GFP |
| Result | No C_{q} | No C_{q} | No C_{q} | C_{q} | Negative |
| Result | No C_{q} | No C_{q} | No C_{q} | No C_{q} | Indeterminate - |
| | | | | | Rerun Assay |

### Inclusivity/Exclusivity Study

A total of 104 isolates of Salmonella were analyzed with the SIMUL-qPCR Salmonella & STEC with GFP Assay (Table 28). Of those 104 isolates, seven were Salmonella enterica subspecies salamae, five were Salmonella enterica subspecies arizonae, five were Salmonella enterica subspecies diarizonae, five were Salmonella enterica subspecies houtenae, five were Salmonella bongori, and the remainder were unique serovars of Salmonella enterica subspecies enterica. Each strain was cultured per the instructions outlined in Example 1.

The testing was performed by sub-culturing isolates from tryptic soy agar (TSA) slants stored in a 2-4° C. refrigerator and placing a loopful of each isolate into EREB and BPW media. Each strain was incubated at 42° C.±1° C. for 10-18 h and then diluted with sterile EREB and BPW media to approximately 100 times the LOD₅₀ of the SIMUL-qPCR Salmonella & STEC with GFP Assay (approximately 10⁵ cfu/mL). Data from the Salmonella inclusivity testing are detailed in Table 28.

A total of 50 isolates of a combination of E. coli strains O157, O26, O45, O103, O111, O121, and O145 were analyzed with the SIMUL-qPCR Salmonella & STEC with GFP Assay using a minimum of 7-8 isolates of each strain type. Each strain was cultured per the instructions outlined in Example 3.

The testing of the STEC strains was performed by sub-culturing from TSA slants stored in a 2-4° C. refrigerator and placing a loopful of each isolate into EREB and BPW media. Each strain was incubated at 42° C.±1° C. for 10-18 h and then diluted with sterile EREB and BPW media to approximately 100 times the LOD₅₀ of the SIMUL-qPCR Salmonella & STEC with GFP Assay (approximately 10⁵ cfu/mL). Data from the STEC inclusivity testing are detailed in Table 29.

A total of 30 isolates of non-Salmonella, non-E. coli species, and E. coli non-STEC (O157, O26, O45, O103, O111, O121, and O145) strains were tested with the SIMUL-qPCR Salmonella & STEC with GFP Assay following the kit instructions. Exclusivity testing was conducted by taking colonies grown on TSA slants stored in a 2-4° C. refrigerator and growing the strains in Brain Heart Infusion (BHI) broth incubated at 37° C.±2° C. for 20-24 h. All strains were tested undiluted after incubation using the SIMUL-qPCR Salmonella & STEC with GFP Assay. Data from the exclusivity testing are detailed in Table 30.

The assay was able to detect all 100 Salmonella strains and all 50 Shiga toxin-producing E. coli strains. The assay also successfully detected the GFP target only in the strains with that marker. The inclusivity also demonstrated that the kit could detect the strains in both BPW and EREB media. The assay also only called positive for Salmonella when Salmonella was the target, while the STEC target was negative, and the same was true for the STEC strains. Exclusivity studies showed that the assay had no false positives in the 30 different non-Salmonella and non-STEC isolates.

### Matrix Study

Leafy greens were obtained from a local grocery store. Dried hemp flower was obtained from a hemp producer in Oregon. Leafy greens and dried hemp flower were prescreened following the SIMUL-qPCR methods for both the Top 7 STEC strains and for Salmonella prior to testing. All materials prescreened were negative for both STEC and Salmonella. The samples were co-inoculated with an STEC strain and a Salmonella strain and then tested by the SIMUL-qPCR STEC/Salmonella with GFP Assay.

Each matrix was artificially contaminated with the indicated strains at two contamination levels; a low level to achieve a fractional response of 5-15 positive test portions out of 20 replicate portions tested (approximately 0.2-2 cfu/test portion), and a high level to achieve five positive test portions out of five replicate portions tested (approximately 5-10 cfu/test portion).

To prepare the materials for testing, each product for each food matrix was aseptically combined into a large sterile sampling bag and hand massaged to thoroughly mix the product together. The product was spiked using strains sub-cultured in BHI at 37±2° C. for 20-24 h. The strains were diluted in Butterfield's Phosphate Diluent (BFD) to achieve the concentrations listed above and then added to the large sampling bags. Samples were mixed well by hand massaging each mixture for a minimum of one minute to create homogenous mixtures. All matrixes were inoculated with UV-BioTAG™ Salmonella enterica subsp. Enterica serovar Typhimurium Sal54 derived from SAL5694 as that strain includes the GFP target. Leafy greens and dried hemp flower were co-inoculated with UV-BioTAG™ Escherichia coli (O157:H7) EC43 derived from FDA ESC1177 (STEC), which also includes the GFP target.

For leafy greens, 25 g portions of each contamination level (including the uncontaminated level) were weighed into a sterile sampling bag. For STEC testing both the candidate and reference methods, an additional 175 g of uninoculated product was added to bring the weight of each portion to 200 g. For Salmonella testing, only the candidate method had an additional 175 g of uninoculated product added to bring the weight of each portion to 200 g. Materials were held at 2-8° C. for 48-72 h to allow for stabilization of the microorganisms in the food environment. For dried hemp flower, 10 g test portions of each contamination level (including the uncontaminated level) were weighed into a sterile filtered stomacher bag and held at room temperature (20-25° C.) for 2 weeks. Each matrix was also tested for aerobic plate count. Counts are outlined in Table 31.

For the candidate method, leafy greens were tested at 10 h and 18 h for single lysates and 12 h and 18 h for pooled lysates, and dried hemp flower was tested at 24 h for both individual and pooled lysates. To prepare the pooled format, test portions were lysed, then 5 μL from each test portion lysate was combined with 5 μL from each of 9 non-inoculated test portion lysates (presumably negative) to create a pool of 10 portions (total volume 50 μL). A pooled sample was prepared to represent each test portion from each level.

For leafy greens, the BAM methods were used as the reference methods for both Salmonella (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety) and STEC (Feng, et al., “Chapter 4A: Diarrheagenic Escherichia coli.” U.S. Food and Drug Administration Bacteriological Analytical Manual (BAM), (2020), which is hereby incorporated by reference in its entirety). For the dried hemp flower, SMPR 2020.002 “Detection of Salmonella species in Cannabis and Cannabis Products”, which is hereby incorporated by reference in its entirety (Salmonella) and SMPR 2020.012 “Detection of Shiga toxin-producing Escherichia coli in Cannabis and Cannabis Products”, which is hereby incorporated by reference in its entirety (STEC) were followed, as there are no reference methods for Cannabis. For leafy greens, for Salmonella detection, a 25 g test portion was added to a sterile container, and then 225 mL of Universal Preenrichment broth was added without homogenization. Test portions were incubated at 35±2° C. for 24±2 h. For STEC detection, 200 g portions were enriched in 450 mL of modified Buffered Peptone Water with Pyruvate (mBPWp) and incubated for 5 h at 37±1° C. before adding 1 mL each of acriflavine, cefsulodin, and vancomycin to the enrichment. The products were then incubated at 42±1° C. for 18-24 h.

Test portions were processed using the SIMUL-qPCR STEC/Salmonella with GFP Assay for the candidate method and the BAM for the reference method. For dried hemp flower, a paired study was conducted (presumptive vs confirmed), since there is no reference method. All leafy green enrichments (SIMUL-qPCR and BAM) were processed for cultural confirmation as directed by BAM Ch. 4A (STEC) and Ch. 5 (Salmonella) regardless of the results obtained by the SIMUL-qPCR method. All hemp flower enrichments (SIMUL-qPCR) were processed for cultural confirmation as directed by SMPRs 2020.002 and 2020.012 regardless of the results obtained by the SIMUL-qPCR method (SMPR 2020.002 “Detection of Salmonella species in Cannabis and Cannabis Products”, which is hereby incorporated by reference in its entirety (Salmonella) and SMPR 2020.012 “Detection of Shiga toxin-producing Escherichia coli in Cannabis and Cannabis Products”, which is hereby incorporated by reference in its entirety).

For Salmonella confirmation, following selective enrichment and plating, colonies were confirmed using the API 20E strips along with group O and poly H antisera for biochemical and serological confirmations. Colonies were also viewed under a 400 nm UV light to confirm the presence of the GFP in the colonies. If fluorescence was hard to see due to competing flora, isolated colonies were obtained and restreaked onto Tryptic Soy Agar as the light background makes it easier to see the colonies fluoresce.

For STEC confirmation, enrichments were streaked to TC-SMAC and Rainbow® agar plates. Colonies were then taken to Trypticase Soy Agar with Yeast Extract (TSAYE) plates for purity. Latex agglutination for both the 0157 antigen and H7 antigen were performed. Any latex-positive colonies were processed for biochemical identification using the VITEK method. Both TC-SMAC plates and Rainbow agar plates were also viewed under a 400 nm UV light to confirm the presence of the GFP in the colonies.

MPN analyses were conducted on the high and low contamination levels. Test portions from the reference method analysis (BAM), plus additional portions from each contamination level were used to create a minimum 5 replicate, 3-level MPN. All MPN results were calculated using the Least Cost Formulations MPN v1.6 calculator program (Virginia Beach, VA). All matrix study data is detailed in Tables 32-43.

Results of the matrix study are outlined in Tables 32-43. Some challenges were encountered during the matrix studies. When trying to achieve fractional levels of dried hemp flower and dried cannabis flower, it was determined through different trials at fractional levels that the media should be pre-warmed at 37° #1° C. and the test portions should be incubated at 42°+1° C. instead of 35° C. Also, during confirmation testing, some enriched test portions had to be re-streaked from the BPW, since the BPW is less selective to remove some of the competing background flora present in the materials, which made it difficult to see the colonies fluoresce. Colonies were streaked onto Tryptic Soy Agar plates for isolation, which made seeing the fluorescence much easier.

PCR results were analyzed for both the Salmonella target, STEC target, and the GFP target. For all results listed, both targets called positive to be included in the number of positives displayed. For confirmation of the candidate method, colonies were viewed under UV light to confirm the presence of the GFP. All samples that confirmed positive for Salmonella and/or STEC also confirmed for the presence of GFP.

Tables 32-35 outline the results of the Salmonella portion of the assay. For leafy greens, 6 out of 20 test portions were presumptive positive for both the individual and pooled lysates for both time points. Six out of 20 confirmed positive for both the reference and candidate methods. For dried hemp flower, 14 out of 20 test portions were presumptive positive for both the individual lysates and 15 out of 20 test poritons were presumptive positive for the pooled lysates. Fifteen out of 20 test portions confirmed positive. As there is no reference method for dried hemp, the tables only outline the presumptive versus confirmed results.

Tables 36-39 outline the results for the STEC portion of the assay. For the individual leafy green test portion lysates, 13 out of 20 samples were presumptive positive on the candidate method at both the 10 and 18 h time points. For the pooled lysates, 13 out of 20 test portions were presumptive positive at the 12 h time point, and 15 test portions were presumptive positive at the 18 h time point. Thirteen out of 20 test portions confirmed positive. The reference method had 14 out of 20 test portions confirm positive, showing no statistically significant differences between the candidate and reference methods at any time points. Also, the candidate method showed no significant difference in individual and pooled lysates results.

For dried hemp flower, 13 out of 20 test portions were presumptive for the candidate method after 24 h. For the pooled test portion lysates, 12 out of 20 test portions were presumptive positive. Thirteen out of 20 test portions confirmed positive. As there is no reference method for dried hemp, the tables only outline the presumptive versus confirmed results.

For these matrixes for both the individual and pooled lysates, no statistically significant differences were found. For all samples, the GFP target also amplified when the Salmonella target or STEC target amplified, demonstrating the assay's ability to detect both the pathogen of interest as well as the GFP target.

### Robustness Study

A robustness study was conducted using leafy greens and dried hemp flower to evaluate the ability of the method to remain unaffected by small variations in method parameters that may occur if the method was performed by an end user. Upper and lower limits of three method parameters were evaluated using a factorial design; enrichment time (9 h and 24 h for leafy greens; 20 h and 30 h for dried hemp flower), volume of extraction reagent 380 μL and 420 μL for leafy greens; 975 μL and 1015 μL for dried hemp flower), and volume of extracted DNA sample (15 μL and 25 μL for both matrixes). The nominal test conditions for the assay are 10 h (leafy greens) or 24 h (dried hemp flower) enrichment time, 400 μL (leafy greens) or 995 μL (dried hemp flower) extraction reagent and 20 μL of extracted sample.

Leafy greens and dried hemp flower were co-inoculated with UV-BioTAG™ Salmonella enterica subsp. Enterica serovar Typhimurium (Sal54 derived from SAL5694) and UV-BioTAG™ E. coli O157:H7 (EC43 derived from FDA ESC1177) at a level to yield fractional results (2-8 positive results out of 10 replicate portions tested). Ten replicate 200 g portions of inoculated leafy greens and 10 replicate 200 g portions of non-inoculated leafy greens were incubated with EREB at 42±1° C. For dried hemp flower, 10 replicate 10 g portions of inoculated dried hemp flower and 10 replicates of 10 g portions of non-inoculated dried hemp flower were incubated with BPW at 42±1° C. After enrichment, aliquots from each enriched test portion were taken at each time point and analyzed using the SIMUL-qPCR Salmonella & STEC with GFP Assay. POD values and 95% confidence intervals were calculated, and data were analyzed for any variance that occurred due to changes in the parameter settings. Results are presented in Tables 40 and 41.

Product Consistency and Stability Studies-Three lots of SIMUL-qPCR Salmonella & STEC with GFP Assay were examined product stability over the 12-month shelf-life period. The study compared lots near expiration (12 month), lots at the middle of the shelf life (6 month), and lots that were recently manufactured (0 month).

Pure cultures of UV-BioTAG™ Salmonella enterica subsp. Enterica serovar Typhimurium Sal54 derived from SAL5694) and UV-BioTAG™ E. coli O157:H7 (EC43 derived from FDA ESC1177) were grown in BPW media at 42±1° C. for 10-18 h. The cultures were diluted in BPW to a level to yield fractional results (2-8 positive results out of 10 replicate portions tested). A pure culture of Citrobacter freundii (ATCC 43864) was also grown in BHI at 37° C. for 20-24 h. This culture was not diluted for the test. Each lot of SIMUL-qPCR Salmonella & STEC with GFP assays were tested with 10 replicates of diluted S. Typhimurium and 10 replicates of undiluted Citrobacter. The samples were blind coded and randomized before being tested.

Test results were analyzed by POD statistical analysis to 95% confidence intervals to determine any variance between lots and time points. Results are shown in Tables 42 and 43.

The results from the robustness study showed that there was no statistically significant difference by POD analysis when small alterations are made to the protocol for either matrix. Increasing or decreasing enrichment time, volume of the extraction reagent, and volume of extracted DNA sample slightly had no effect on the performance of the Salmonella & STEC with GFP Assay.

For the real-time product consistency and stability study, there was no statistically significant differences by POD analysis between the recently manufactured lots and those halfway through the expiration period or the recently manufactured lots and those nearing expiration. The study verified the one-year shelf life of the SIMUL-qPCR Salmonella & STEC with GFP Assay.

### Independent Lab Study

Samples of dried Cannabis flower were sent to an independent laboratory for analysis. Dense buds of dried Cannabis flower (>0.3% THC) were obtained by the independent laboratory. Inoculation procedure was followed as outlined in AOAC's Appendix J microbiology validation guidelines (Official Methods of Analysis, 21st Ed., Appendix J, AOAC INTERNATIONAL, Rockville, MD (2019), which is hereby incorporated by reference in its entirety). Prior to inoculation, a total aerobic plate count was performed following Maturin and Peeler, “BAM Chapter 3: Aerobic Plate Count,” U.S. Food and Drug Administration Bacteriological Analytical Manual, which is hereby incorporated by reference in its entirety, and matrix was pre-screened for the target organisms as outlined in SMPR 2020.002 (for Salmonella) and SMPR 2020.012 (for STEC) (SMPR 2020.002 “Detection of Salmonella species in Cannabis and Cannabis Products”, which is hereby incorporated by reference in its entirety (Salmonella) and SMPR 2020.012 “Detection of Shiga toxin-producing Escherichia coli in Cannabis and Cannabis Products”, which is hereby incorporated by reference in its entirety). No natural contamination was found, so artificial contamination was required. The dried flower materials were broken up by hand, placed into large plastic stomacher-type bags, and then hand massaged further to create small particles. The bags were shaken to mix the particles. A portion of each material was set aside prior to inoculation to serve as the non-inoculated level (0 cfu/test portion). Dried cannabis flower was co-inoculated with both Salmonella and STEC target organisms. Certified reference materials in the form of lyophilized pellets (S. Typhimurium, ATCC 14028, and E. coli O157:H7, ATCC 43895), were crushed into powdered Non-fat Dry Milk (NFDM) and serial dilutions of the inoculated NFDM were made and a three-level MPN was performed to determine the cfu/pellet.

A large container of non-contaminated matrix was mixed with the inoculated NFDM by hand massaging and agitating with the sterile bag to mix. The inoculated material was allowed to equilibrate for two weeks at room temperature (20-25° C.) prior to testing. An MPN was performed using the same AOAC PTM certified methods that were used for the screening at a time point in the two-week hold time to determine the inoculum concentration in the matrix prior to validation testing. The dried cannabis flower materials (>0.3% THC) were adjusted by either adding non-inoculated material to create a lower contamination level, or by adding the more inoculum to create a higher contamination level. The bags were hand massaged and agitated after any adjustments to ensure homogeneity.

In this study, dried cannabis flower (>0.3% THC) was tested at a 10 g test portion size. Matrix was analyzed at three levels of artificial contamination: non-inoculated (0 cfu/test portion), low level (˜2 cfu/test portion), and high level (˜10 cfu/test portion). A 3-level MPN was performed on the day of testing for both the low and high inoculation levels. For the low level, 5×20 g test portions, 5×10 g test portions, and 5×2 g test portions were used as the large, medium, and small test portions, respectively. For the high level, 5×10 g test portions, 5×5 g test portions, and 5×1 g test portions were used. Test portion preparation, analysis, and presumptive positive confirmations were performed according to SMPR 2020.002 for Salmonella, and according to SMPR 2020.012 for STEC.

Ten grams of dried cannabis flower was weighed out into a 24 oz filter bag and 90 mL of pre-warmed (37°±1° C.) BPW was added to the bag. Test portions were hand massaged for 30 s to homogenize and incubated at 42°±1° C. for 24 h. The lysis procedure outlined above was followed and run on the SIMUL-qPCR Salmonella & STEC with GFP Assay. Once results were completed, all test portions were confirmed for both Salmonella and STEC following the procedures outlined in the BAM protocol. The independent laboratory did not have a strain tagged with GFP, and all test portions did not amplify for that target. Results are included in Tables 32 and 36. The independent laboratory did not test the lysates in a pooled format. However, the laboratory did send the lysates to the method developer, who pooled the lysates and tested them according to the method developer protocol. The lysates were shipped frozen priority overnight to the laboratory, who then thawed, pooled, and tested the lysates the same day as receipt. Those results are included in Tables 34 and 38.

The independent laboratory study data are included in Tables 32 and 36. The independent laboratory did not have the GFP target strain to evaluate that portion of the assay. They also did not pool the lysates. However, TEQ Analytical Labs did ship the lysates to the method developer, who was then able to pool the lysates and evaluate those results. The pooled data is included in Tables 34 and 38.

According to the independent laboratory, the SIMUL-qPCR Salmonella & STEC with GFP Assay successfully detected the target Salmonella and STEC species in dried cannabis flower (>0.3% THC) at a 10 g test portion size. For Salmonella, the individual test portion lysates had 15 presumptive positive results, and 14 confirmed positive results. For the pooled lysates set, there were 14 presumptive positive results and 14 confirmed positive. For STEC, both the individual and pooled test portion lysates had 10 presumptive positive results which all confirmed positive. Difference in POD analysis for the presumptive versus confirmed positive results showed that no statistically significant differences were detected, with all ranges of the 95% confidence intervals containing the zero (0) point.

According to the independent laboratory, processing the test portions for these assays was very user friendly with a transfer of enrichment into aliquoted lysis buffer, a heat and cool step of the lysis, and then transfer into pre-aliquoted lyophilized pellets in PCR wells. Despite the small capacity of the MyGo Pro32 instrument, the short run time and co-detection of both target analytes allows for high sample throughput. Presumptive results are indicated with a Cq result; however, review is necessary by the analyst to determine if the Cq has sigmoidal or the beginning of sigmoidal amplification, corresponding to a true presumptive positive.

Upon interpretation, some of the results were different than that of the software programs initial result. For this study, the software determined two false negative results, but as the curves were evaluated, they were recognized to be true negative results with typical curve patterns.

The SIMUL-qPCR Salmonella and STEC with GFP Assay allows users to obtain presumptive positive results after 24 h of incubation and one to two hours of processing, assay run time, and interpretation of results.

For the pooled lysates of the dried cannabis flower, both the Salmonella and STEC presumptive results matched the confirmed results (14 for Salmonella and 10 for STEC). Because the independent laboratory inoculated with strains that did not contain GFP, the GFP target did not amplify for the pooled lysates.

### CONCLUSION

This data confirmed that the SIMUL-qPCR Salmonella & STEC with GFP Assay was suitable for detecting Salmonella species and the Top 7 STEC species in leafy greens, dried hemp flower (≤0.3% THC), and dried cannabis flower (>0.3% THC), as well as GFP tagged strains. Furthermore, the data confirmed that the method was valid for up to 10 pooled lysates. POD analysis showed that no statistically significant differences were detected between the candidate method presumptive and confirmed results, and between the candidate method and the BAM Chapter 4A (Feng, et al., “Chapter 4A: Diarrheagenic Escherichia coli.” U.S. Food and Drug Administration Bacteriological Analytical Manual (BAM), (2020), which is hereby incorporated by reference in its entirety), BAM Chapter 5 (Andrews et al., “Bacteriological Analytical Manual (BAM) Chapter 5: Salmonella.” U.S. Food and Drug Administration Bacteriological Analytical Manual 110 (2018), which is hereby incorporated by reference in its entirety) reference method results for the matrixes analyzed. Small variations in parameters will not affect the validity of the results obtained by the method, and the assay collection is proven to perform adequately during its one-year shelf life. Also, an independent laboratory validated that the method is suitable for detection of Salmonella and STEC strains in dried Cannabis flower. Finally, the study proved that the assay could detect a GFP target if a strain is tagged with the GFP, which can add value to laboratories in limiting time and labor in investigations into possible internal cross-contamination issues.

TABLE 28
Inclusivity List - Salmonella Strains

| | | | | | | SIMUL- | SIMUL- | |
|---|---|---|---|---|---|---|---|---|
| | | | | | | qPCR | qPCR | |
| | | | | | | Result: | Result: | |
| No. | Genus | Species | Serovar | Source | Origin | Salmonella | STEC | GFP |
| 1 | Salmonella | enterica subsp. | Typhimurium | FDA SAL 5694^{a} | Unknown | +^{f} | − | + |
| | | enterica | | | | | | |
| 2 | Salmonella | enterica subsp. | 1, 40:g, z51:- | SGSC^{b} 3120 | Unknown, Tonga | + | − | − |
| | | houtenae | | | | | | |
| 3 | Salmonella | enterica subsp. | 11:b:e, n, x | SGSC 3118 | Unknown | + | − | − |
| | | indica | | | | | | |
| 4 | Salmonella | enterica subsp. | 16:z4, z32:— | SGSC 3086 | Human, Illinois | + | − | − |
| | | houtenae | | | | | | |
| 5 | Salmonella | enterica subsp. | 38[k]:z35:— | SGSC 3069 | Human, | + | − | − |
| | | diarizonae | | | California | | | |
| 6 | Salmonella | enterica subsp. VII | 40:z4, z24:— | SGSC 3121 | Human, Florida | + | − | − |
| 7 | Salmonella | enterica subsp. | 42:f:g, t:— | SGSC 3047 | Unknown | + | − | − |
| | | salamae | | | | | | |
| 8 | Salmonella | enterica subsp. | 45:a:e, n, x | SGSC 3116 | Unknown, India | + | − | − |
| | | indica | | | | | | |
| 9 | Salmonella | enterica subsp. IV | 45a, b:g, z32:— | SGSC 3074 | Animal, Canal | + | − | − |
| | | | | | Zone | | | |
| 10 | Salmonella | enterica subsp. | Agona (B) | SGSC 2458 | Unknown, Peru | + | − | − |
| | | enterica | | | | | | |
| 11 | Salmonella | enterica subsp. | Anatum (E₁) | SGSC 2459 | Human, | + | − | − |
| | | enterica | | | Washington | | | |
| 12 | Salmonella | enterica subsp. | 62:z4, z23:— | SGSC 3061 | Corn snake, | + | − | − |
| | | arizonae | | | Oregon | | | |
| 13 | Salmonella | enterica subsp. | 62:z36:— | SGSC 3063 | Human, | + | − | − |
| | | arizonae | | | California | | | |
| 14 | Salmonella | enterica subsp. | 51:z(4), z(23):(—) | SGSC 2426 | Unknown | + | − | − |
| | | arizonae | | | | | | |
| 15 | Salmonella | enterica subsp. | II | SGSC 2415 | Unknown | + | − | − |
| | | salamae | 17:g, t:[e, n, x, z₁₅] | | | | | |
| 16 | Salmonella | enterica subsp. | II | SGSC 2414 | Unknown | + | − | − |
| | | salamae | 6, 7:b:e, n, x:z₄₂ | | | | | |
| 17 | Salmonella | bongori | 48:z35:— | SGSC 2556 | Unknown | + | − | − |
| 18 | Salmonella | bongori | 66:z41:— | SGSC 3100 | Frog | + | − | − |
| 19 | Salmonella | bongori | 48:z41:— | SGSC 3103 | Parakeet, USA | + | − | − |
| 20 | Salmonella | bongori | 66:z(41):(—) | SGSC 2429 | Unknown | + | − | − |
| 21 | Salmonella | bongori | 66:z41:— | SGSC 2557 | Unknown | + | − | − |
| 22 | Salmonella | enterica subsp. | Choleraesuis | SGSC 2461 | Swine, Minnesota | + | − | − |
| | | enterica | | | | | | |
| 23 | Salmonella | enterica subsp. | Decatur | SGSC 2465 | Unknown, France | + | − | − |
| | | enterica | | | | | | |
| 24 | Salmonella | enterica subsp. | Derby (B) | SGSC 2466 | Avian, Oklahoma | + | − | − |
| | | enterica | | | | | | |
| 25 | Salmonella | enterica subsp. | 6, 7:1, v:z(53) | SGSC 2427 | Unknown | + | − | − |
| | | diarizonae | | | | | | |
| 26 | Salmonella | enterica subsp. | Dublin (D1) | SGSC 3611 | Unknown | + | − | − |
| | | enterica | | | | | | |
| 27 | Salmonella | enterica subsp. | Bredeney (B) | SGSC 4931 | Unknown | + | − | − |
| | | enterica | | | | | | |
| 28 | Salmonella | enterica subsp. | Duisberg | SGSC 2472 | Unknown, | + | − | − |
| | | enterica | | | Scotland | | | |
| 29 | Salmonella | enterica subsp. | Emek (C₃) | SGSC 2477 | Unknown, Israel | + | − | − |
| | | enterica | | | | | | |
| 30 | Salmonella | enterica subsp. | Newport (C₂) | ATCC^{c} 6962 | Food poisoning | + | − | − |
| | | enterica | | | fatality | | | |
| 31 | Salmonella | enterica subsp. | Enteritidis (D₁) | ATCC 13076 | Unknown | + | − | − |
| | | enterica | | | | | | |
| 32 | Salmonella | enterica subsp. | Montevideo | ATCC 8387 | Unknown | + | − | − |
| | | enterica | (C₁) | | | | | |
| 33 | Salmonella | enterica subsp. | Schwarzengrund | BEI^{d} NR-28796 | Human, Oregon | + | − | − |
| | | enterica | (B) | | | | | |
| 34 | Salmonella | enterica subsp. | Typhi (D₁) | BEI NR-513 | USA | + | − | − |
| | | enterica | | | | | | |
| 35 | Salmonella | enterica subsp. | Typhimurium | USDA ERRC^{e} | Ames, B. N. (U | + | − | − |
| | | enterica | (B) | B-4211 | California, Davis, | | | |
| | | | | | CA) | | | |
| 36 | Salmonella | enterica subsp. | Poona (G₁) | SGSC 4934 | Unknown | + | − | − |
| | | enterica | | | | | | |
| 37 | Salmonella | enterica subsp. | Blockley C₂) | SGSC 4935 | Unknown | + | − | − |
| | | enterica | | | | | | |
| 38 | Salmonella | enterica subsp. | Livingstone | SGSC 4937 | Unknown | + | − | − |
| | | enterica | (C₁) | | | | | |
| 39 | Salmonella | enterica subsp. | Hvittingfoss (I) | SGSC 4947 | Unknown | + | − | − |
| | | enterica | | | | | | |
| 40 | Salmonella | enterica subsp. | Bareilly (C₁) | SGSC 4949 | Unknown | + | − | − |
| | | enterica | | | | | | |
| 41 | Salmonella | enterica subsp. | Mbandaka (C₁) | SGSC 2958 | Unknown | + | − | − |
| | | enterica | | | | | | |
| 42 | Salmonella | enterica subsp. | Setubal (60) | SGSC 2567 | Unknown, | + | − | − |
| | | enterica | | | Mississippi | | | |
| 43 | Salmonella | enterica subsp. | Ferlac (VI | SGSC 2581 | Unknown | + | − | − |
| | | indica | 1, 6, 14, 25) | | | | | |
| 44 | Salmonella | enterica subsp. | Flint (IV 50) | SGSC 2554 | Unknown | + | − | − |
| | | enterica | | | | | | |
| 45 | Salmonella | enterica subsp. | Gallinarum | SGSC 2478 | Human, | + | − | − |
| | | enterica | (D₁) | | Connecticut | | | |
| 46 | Salmonella | enterica subsp. | Haifa (B) | SGSC 2479 | Unknown, | + | − | − |
| | | enterica | | | Scotland | | | |
| 47 | Salmonella | enterica subsp. | Heidelberg (B) | SGSC 2480 | Chicken, | + | − | − |
| | | enterica | | | Pennsylvania | | | |
| 48 | Salmonella | enterica subsp. | 45:g, z(51):(—) | SGSC 2428 | Unknown | + | − | − |
| | | houtenae | | | | | | |
| 49 | Salmonella | enterica subsp. | Indiana (B) | SGSC 2482 | Unknown, | + | − | − |
| | | enterica | | | Scotland | | | |
| 50 | Salmonella | enterica subsp. | 1, 6, 14, 25:a:e, n, | SGSC 2430 | Unknown | + | − | − |
| | | indica | X | | | | | |
| 51 | Salmonella | enterica subsp. | Infantis (C₁) | SGSC 2483 | Human, North | + | − | − |
| | | enterica | | | Carolina | | | |
| 52 | Salmonella | enterica subsp. | Argentina (IV | SGSC 2555 | Unknown | + | − | − |
| | | enterica | 6, 7) | | | | | |
| 53 | Salmonella | enterica subsp. | Paratyphi B | SGSC 4951 | Unknown | + | − | − |
| | | enterica | var. Java | | | | | |
| 54 | Salmonella | enterica subsp. | Krefeld (E₄) | SGSC 4945 | Unknown, France | + | − | − |
| | | enterica | | | | | | |
| 55 | Salmonella | enterica subsp. | Malawi (66) | SGSC 2577 | Unknown | + | − | − |
| | | enterica | | | | | | |
| 56 | Salmonella | enterica subsp. | Maregrosso (V | SGSC 2578 | Unknown | + | − | − |
| | | enterica | 66) | | | | | |
| 57 | Salmonella | enterica subsp. | Miami (D₁) | SGSC 2486 | Human, French | + | − | − |
| | | enterica | | | Guiana | | | |
| 58 | Salmonella | enterica subsp. | 58:d:z | SGSC 3039 | Human, | + | − | − |
| | | salamae | | | Massachusetts | | | |
| 59 | Salmonella | enterica subsp. | Muenchen (C₂) | SGSC 2490 | Human, France | + | − | − |
| | | enterica | | | | | | |
| 60 | Salmonella | enterica subsp. | Naestved (D₁) | SGSC 3612 | Unknown | + | − | − |
| | | enterica | | | | | | |
| 61 | Salmonella | enterica subsp. | Newport (C₂) | SGSC 2493 | Human, North | + | − | − |
| | | enterica | | | Carolina | | | |
| 62 | Salmonella | enterica subsp. | 50:k:z | SGSC 3068 | Human, Oregon | + | − | − |
| | | diarizonae | | | | | | |
| 63 | Salmonella | enterica subsp. | Ohio (C₁) | SGSC 4943 | Unknown | + | − | − |
| | | enterica | | | | | | |
| 64 | Salmonella | enterica subsp. | Panama (D₁) | SGSC 3583 | Unknown | + | − | − |
| | | enterica | | | | | | |
| 65 | Salmonella | enterica subsp. | Paratyphi A | SGSC 4568 | Unknown | + | − | − |
| | | enterica | | | | | | |
| 66 | Salmonella | enterica subsp. | Paratyphi B | SGSC 4567 | Unknown | + | − | − |
| | | enterica | | | | | | |
| 67 | Salmonella | enterica subsp. | Paratyphi C | SGSC 2290 | Unknown | + | − | − |
| | | enterica | | | | | | |
| 68 | Salmonella | enterica subsp. | Oranienburg | BEI NR-171 | Human, Illinois | + | − | − |
| | | enterica | (C₁) | | | | | |
| 69 | Salmonella | enterica subsp. | Javiana (D₁) | BEI NR-4296 | Human, | + | − | − |
| | | enterica | | | Pennsylvania | | | |
| 70 | Salmonella | enterica subsp. | Pullorum (D₁) | SGSC 2508 | Unknown, | + | − | − |
| | | enterica | | | Germany | | | |
| 71 | Salmonella | enterica subsp. | Reading (B) | SGSC 2510 | Unknown, | + | − | − |
| | | enterica | | | Scotland | | | |
| 72 | Salmonella | enterica subsp. | Rissen (C₁) | SGSC 4941 | Unknown | + | − | − |
| | | enterica | | | | | | |
| 73 | Salmonella | enterica subsp. | Rubislaw (11) | SGSC 2511 | Unknown, | + | − | − |
| | | enterica | | | Laboratory Strain | | | |
| 74 | Salmonella | enterica subsp. | S.4, 5, 12:i:— | SGSC 4956 | Unknown | + | − | − |
| | | enterica | | | | | | |
| 75 | Salmonella | enterica subsp. | Saintpaul (B) | SGSC 2512 | Human, Texas | + | − | − |
| | | enterica | | | | | | |
| 76 | Salmonella | enterica subsp. | 1, 9, 12:1, w:e, n, | SGSC 2425 | Unknown | + | − | − |
| | | salamae | x | | | | | |
| 77 | Salmonella | enterica subsp. | Abortusovis | BEI NR-13556 | Unknown, France | + | − | − |
| | | enterica | (B) | | | | | |
| 78 | Salmonella | enterica subsp. | Sendai (D₁) | SGSC 2515 | Human, | + | − | − |
| | | enterica | | | California | | | |
| 79 | Salmonella | enterica subsp. | Senftenberg | SGSC 2516 | Chicken, | + | − | − |
| | | enterica | (E₄) | | Maryland | | | |
| 80 | Salmonella | spp. | 44:z39:— | SGSC 3105 | Food, Ghana | + | − | − |
| 81 | Salmonella | enterica subsp. | Stanley (B) | SGSC 2517 | Unknown, | + | − | − |
| | | enterica | | | Scotland | | | |
| 82 | Salmonella | enterica subsp. | Stanleyville | SGSC 2518 | Unknown, | + | − | − |
| | | enterica | (B) | | Scotland | | | |
| 83 | Salmonella | enterica subsp. | Tennessee (C₁) | SGSC 4939 | Unknown | + | − | − |
| | | enterica | | | | | | |
| 84 | Salmonella | enterica subsp. | Thompson (C₁) | SGSC 2519 | Human, Florida | + | − | − |
| | | enterica | | | | | | |
| 85 | Salmonella | enterica subsp. | Hadar (C₂) | BEI NR-28799 | Human, | + | − | − |
| | | enterica | | | Massachusetts | | | |
| 86 | Salmonella | enterica subsp. | Virchow (C₁) | BEI NR-28801 | Human, | + | − | − |
| | | enterica | | | Connecticut | | | |
| 87 | Salmonella | enterica subsp. | Kentucky (C₃) | BEI NR-28795 | Human, | + | − | − |
| | | enterica | | | Wisconsin | | | |
| 88 | Salmonella | enterica subsp. | Typhisuis (B) | SGSC 2526 | Swine, California | + | − | − |
| | | enterica | | | | | | |
| 89 | Salmonella | enterica subsp. | Vrindaban (VI | SGSC 2582 | Unknown | + | − | − |
| | | enterica | 45) | | | | | |
| 90 | Salmonella | enterica subsp. | Wassenaar (IV | SGSC 2576 | Unknown | + | − | − |
| | | enterica | 50) | | | | | |
| 91 | Salmonella | enterica subsp. | Weltevreden | SGSC 4929 | Unknown | + | − | − |
| | | enterica | (E₁) | | | | | |
| 92 | Salmonella | enterica subsp. | Wien (B) | SGSC 2529 | Human, France | + | − | − |
| | | enterica | | | | | | |
| 93 | Salmonella | enterica subsp. | Brandenberg | SGSC 2460 | Unknown, | + | − | − |
| | | enterica | (B) | | Scotland | | | |
| 94 | Salmonella | enterica subsp. | 45:a:e, n, x | ATCC BAA- | India | + | − | − |
| | | indica | | 1578 | | | | |
| 95 | Salmonella | enterica subsp. | 6, 7:1, v:z53 | ATCC 43973 | Unknown | + | − | − |
| | | diarizonae | | | | | | |
| 96 | Salmonella | enterica subsp. | 1, 9, 12, [Vi]:g, p:— | SGSC 2471 | Unknown, | + | − | − |
| | | dublin | | | Thailand | | | |
| 97 | Salmonella | enterica subsp. | 11:z4, z23: | ATCC 15788 | Water, Bonaire | + | − | − |
| | | houtenae | | | | | | |
| 98 | Salmonella | enterica subsp. | 9, 46:z4z24:z39:z42 | ATCC 15793 | Lizard, | + | − | − |
| | | salamae | | | Netherlands | | | |
| 99 | Salmonella | enterica subsp. | 55:k:z39 | ATCC 700148 | Unknown | + | − | − |
| | | salamae | | | | | | |
| 100 | Salmonella | enterica subsp. wien | 1, 4, 12, 27:b:l, w | SGSC 2528 | Human, France | + | − | − |
| 101 | Salmonella | enterica subsp. | IIIb | ATCC 12325 | CDC | + | − | − |
| | | diarizonae | 47:1:z53:z57 | | | | | |
| 102 | Salmonella | enterica subsp. | 18:z4, z32: | SGSC 2416 | Unknown | + | − | − |
| | | arizonae | | | | | | |
| 103 | Salmonella | enterica subsp. | 50:r:z | SGSC 2417 | Unknown | + | − | − |
| | | arizonae | | | | | | |
| 104 | Salmonella | enterica subsp. | 45:g, z51 :— | ATCC 43974 | Unknown | + | − | − |
| | | houtenae | | | | | | |

^{a}GFP Strain derived from FDA SAL5694, Food and Drug Administration, Silver Spring, MD.
^{b}Salmonella Genetic Stock Centre, University of Calgary, Canada.
^{c}American Type Culture Collection, Manassas, VA.
^{d}BEI Resources, Manassas, VA.
^{e}United States Department of Agriculture Eastern Regional Research Center, Wyndmoor, PA.
^{f}+ and − indicates a positive or negative result obtained, respectively.

TABLE 29
Inclusivity List - Top 7 STEC Strains

| | | | | | | | SIMUL- | SIMUL- | |
|---|---|---|---|---|---|---|---|---|---|
| | | | | | | | qPCR | qPCR | |
| | | | | | | | Result: | Result: | |
| No. | Species | Serotype | stx | eae | Source | Origin | Salmonella | STEC | GFP |
| 1 | E. coli | O157:H7 | Pos | Pos | FDA ESC1177^{a} | Unknown | − | +^{f} | + |
| 2 | E. coli | O157:H7 | Pos | Pos | ATCC^{b} 43895 | Raw hamburger meat | − | + | − |
| 3 | E. coli | O157:H7 | Pos | Pos | PSU ECRC^{c} 99.0238 | Deer | − | + | − |
| 4 | E. coli | O157:H7 | Pos | Pos | PSU ECRC 10.2268 | Cow | − | + | − |
| 5 | E. coli | O157:H7 | Pos | Pos | ATCC 35150 | Human | − | + | − |
| 6 | E. coli | O157:H7 | Pos | Pos | PSU ECRC 10.2550 | Unknown | − | + | − |
| 7 | E. coli | O157:H7 | Pos | Pos | BEI^{d} NR-8 | Human | − | + | − |
| 8 | E. coli | O157:H7 | Pos | Pos | BEI NR-7 | Human | − | + | − |
| 9 | E. coli | O103 | Pos | Pos | MSU^{e} TW01675 | Human | − | + | − |
| 10 | E. coli | O103 | Pos | Pos | MSU TW07881 | Human | − | + | − |
| 11 | E. coli | O103 | Pos | Pos | MSU TW07975 | Human | − | + | − |
| 12 | E. coli | O103 | Pos | Pos | MSU TW07990 | Human | − | + | − |
| 13 | E. coli | O103 | Pos | Pos | MSU TW04162 | Human | − | + | − |
| 14 | E. coli | O103 | Pos | Pos | PSU ECRC 17.2641 | Unknown | − | + | − |
| 15 | E. coli | O103:H2 | Pos | Pos | BEI NR-17625 | Horse | − | + | − |
| 16 | E. coli | O103:H2 | Pos | Pos | BEI NR-17626 | Human | − | + | − |
| 17 | E. coli | O111 | Pos | Pos | MSU TW07926 | Human | − | + | − |
| 18 | E. coli | O111 | Pos | Pos | MSU TW07813 | Human | − | + | − |
| 19 | E. coli | O111 | Pos | Pos | MSU TW05608 | Human | − | + | − |
| 20 | E. coli | O111 | Pos | Pos | MSU TW04257 | Human | − | + | − |
| 21 | E. coli | O111 | Pos | Pos | MSU TW07870 | Cow | − | + | − |
| 22 | E. coli | O111 | Pos | Pos | MSU TW05150 | Cow | − | + | − |
| 23 | E. coli | O111 | Pos | Pos | PSU ECRC 0.1079 | Cow | − | + | − |
| 24 | E. coli | O121 | Pos | Pos | MSU TW08004 | Human | − | + | − |
| 25 | E. coli | O121 | Pos | Pos | PSU ECRC 17.2643 | Unknown | − | + | − |
| 26 | E. coli | O121 | Pos | Pos | MSU TW08039 | Human | − | + | − |
| 27 | E. coli | O121 | Pos | Pos | MSU TW07972 | Human | − | + | − |
| 28 | E. coli | O121 | Pos | Pos | MSU TW08969 | Human | − | + | − |
| 29 | E. coli | O121 | Pos | Pos | MSU TW08970 | Human | − | + | − |
| 30 | E. coli | O121:H19 | Pos | Pos | BEI NR-17630 | Human | − | + | − |
| 31 | E. coli | O145 | Pos | Pos | MSU TW07596 | Human | − | + | − |
| 32 | E. coli | O145 | Pos | Pos | MSU TW03064 | Rabbit | − | + | − |
| 33 | E. coli | O145 | Pos | Pos | MSU TW08087 | Human | − | + | − |
| 34 | E. coli | O145 | Pos | Pos | MSU TW09153 | Human | − | + | − |
| 35 | E. coli | O145 | Pos | Pos | PSU ECRC 4.0967 | Rabbit | − | + | − |
| 36 | E. coli | O145 | Pos | Pos | MSU TW09356 | Human | − | + | − |
| 37 | E. coli | O145:H2 | Pos | Pos | BEI NR-17633 | Rabbit | − | + | − |
| 38 | E. coli | O26 | Pos | Pos | MSU TW08998 | Human | − | + | − |
| 39 | E. coli | O26 | Pos | Pos | PSU ECRC 5.2217 | Human | − | + | − |
| 40 | E. coli | O26 | Pos | Pos | MSU TW09156 | Human | − | + | − |
| 41 | E. coli | O26 | Pos | Pos | MSU TW04270 | Human | − | + | − |
| 42 | E. coli | O26 | Pos | Pos | MSU TW07936 | Human | − | + | − |
| 43 | E. coli | O26 | Pos | Pos | MSU TW07948 | Human | − | + | − |
| 44 | E. coli | O26 | Pos | Pos | PSU ECRC 6.1592 | Unknown | − | + | − |
| 45 | E. coli | O45 | Pos | Pos | MSU TW00965 | Human | − | + | − |
| 46 | E. coli | O45 | Pos | Pos | MSU TW03068 | Cow | − | + | − |
| 47 | E. coli | O45 | Pos | Pos | PSU ECRC 11.1079 | Goat | − | + | − |
| 48 | E. coli | O45 | Pos | Pos | MSU TW03070 | Cow | − | + | − |
| 49 | E. coli | O45 | Pos | Pos | MSU TW01589 | Cow | − | + | − |
| 50 | E. coli | O45 | Pos | Pos | MSU TW07947 | Human | − | + | − |
| 51 | E. coli | O45 | Pos | Pos | MSU TW09183 | Human | − | + | − |

^{a}GFP Strain derived from FDA SAL5694, Food and Drug Administration, Silver Spring, MD.
^{b}American Type Culture Collection, Manassas, VA.
^{c}The Pennsylvania State University E. coli Reference Center, University Park, PA.
^{d}BEI Resources, Manassas, VA.
^{e}Michigan State University STEC Center, East Lansing, MI.
^{f}+ and − indicates a positive or negative result obtained, respectively.

TABLE 30
Exclusivity List - Non-Salmonella and Non-STEC Strains

| | | | | | | SIMUL- | SIMUL- |
|---|---|---|---|---|---|---|---|
| | | | | | SIMUL-qPCR | qPCR | qPCR |
| | | | | | Salmonella | STEC | GFP |
| No. | Genus | Species | Source | Origin | Result | Result | Result |
| 1 | Bacillus | cereus | BEI^{a} NR-2488 | Blood | −^{e} | − | − |
| 2 | Bacillus | subtilis | BEI NR-607 | Unknown | − | − | − |
| 3 | Bacillus | pumilus | BEI NR-605 | Unknown | − | − | − |
| 4 | Carnobacterium | divergens | ATCC^{b} 35677 | Vacuum-packed | − | − | − |
| | | | | minced beef | | | |
| 5 | Citrobacter | freundii | ATCC 43864 | Unknown | − | − | − |
| 6 | Citrobacter | koseri | SGSC^{c} 5610 | Clinical | − | − | − |
| 7 | Ewingella | americana | SGSC 5640 | Human feces | − | − | − |
| 8 | Enterobacter | cloacae | SGSC 5330 | Unknown | − | − | − |
| 9 | Enterobacter | aerogenes | SGSC 5347 | Unknown | − | − | − |
| 10 | Enterobacter | taylorae | SGSC 5283 | Unknown | − | − | − |
| 11 | Escherichia | coli | ATCC 10536 | Unknown | − | − | − |
| 12 | Escherichia | coli01:K1:H(−) | SGSC 2811 | Unknown | − | − | − |
| 13 | Escherichia | fergusoni | SGSC 5718 | Human feces | − | − | − |
| 14 | Klebsiella | oxytoca | SGSC 5366 | Unknown | − | − | − |
| 15 | Klebsiella | pneumoniae subsp. | SGSC 5926 | Unknown | − | − | − |
| | | pneumoniae | | | | | |
| 16 | Klebsiella | planticola | SGSC 5929 | Radish root | − | − | − |
| 17 | Pseudomonas | aeruginosa | BEI NR-48982 | Human | − | − | − |
| 18 | Proteus | mirabilis | SGSC 5445 | Unknown | − | − | I |
| 19 | Providencia | stuartii | SGSC 5639 | Clinical | − | − | − |
| 20 | Serratia | marcescens | SGSC 5354 | Unknown | − | − | − |
| 21 | Serratia | odorifera | SGSC 5720 | Sputum | − | − | − |
| 22 | Shigella | flexneri | SGSC 5577 | Unknown | − | − | − |
| 23 | Streptococcus | pyogenes | BEI NR-51272 | Human | | − | − |
| 24 | Staphylococcus | aureus | BEI NR-46412 | Urine | − | − | − |
| 25 | Streptococcus | bovis | USDA^{d} ERRC B- | Diseased bovine tissue | − | − | − |
| | | | 23788 | | | | |
| 26 | Yersinia | enterocolitica | USDA ERRC B- | Ground beef | − | − | − |
| | | | 41479 | | | | |
| 27 | Listeria | monocytogenes 4c | BEI NR-111 | Chicken | − | − | − |
| 28 | Listeria | marthii | BEI NR-9582 | River water | − | − | − |
| 29 | Escherichia | blattae | SGSC 5712 | Cockroach | − | − | − |
| 30 | Escherichia | hermanii | SGSC 5715 | Human toe | − | − | − |

^{a}BEI Resources, Manassas, VA.
^{b}American Type Culture Collection, Manassas, VA.
^{c}Salmonella Genetic Stock Centre, University of Calgary, Canada.
^{d}United States Department of Agriculture Eastern Regional Research Center, Windsor, PA.
^{e}+ and − indicates a positive or negative result obtained, respectively.

TABLE 31
Aerobic Plate Count Results of Matrixes

| No | Food | Aerobic Plate Count | |
|---|---|---|---|
| 1 | Leafy Greens | 340 | cfu/g |
| 2 | Dried Hemp Flower | 20 | cfu/g |
| 3 | Dried Cannabis Flower | 5,000 | cfu/g |

TABLE 32
SIMUL-qPCR Salmonella & STEC with GFP Assay Presumptive vs. Confirmed Salmonella Results
- Individual lysates for leafy greens, dried hemp flower, and dried cannabis flower

| | | | | SIMUL-qPCR Salmonella & | | | SIMUL-qPCR Salmonella | | | | |
|---|---|---|---|---|---|---|---|---|---|---|---|
| | | | | STEC with GFP | | | & STEC with GFP | | | | |
| | | MPN^{a}/Test | | Presumptive | | | Confirmede | | | | |
| Matrix | Strain | portion | N^{b} | x^{c} | POD_{CP}^{d} | 95% CI | c | POD_{CC}^{f} | 95% CI | dPOD_{CP}^{g} | 95% CI^{h} |
| Leafy | Salmonella | N/A^{j} | 5 | 0 | 0.00 | 0.00, 0.43 | 0 | 0.00 | 0.00, 0.43 | 0.00 | −0.47, |
| Greens | Typhimurium | | | | | | | | | | 0.47 |
| 10 h | SAL54¹ | 1.097 | 20 | 6 | 0.30 | 0.15, 0.52 | 6 | 0.30 | 0.15, 0.52 | 0.00 | −0.13, |
| | | (0.69, 1.86) | | | | | | | | | 0.13 |
| | | ^{k} | 5 | 5 | 1.00 | 0.57, 1.00 | 5 | 1.00 | 0.57, 1.00 | 0.00 | −0.47, |
| | | | | | | | | | | | 0.47 |
| Leafy | Salmonella | N/A | 5 | 0 | 0.00 | 0.00, 0.43 | 0 | 0.00 | 0.00, 0.43 | 0.00 | −0.47, |
| Greens | Typhimurium | | | | | | | | | | 0.47 |
| 18 h | SAL54 | 1.097 | 20 | 6 | 0.30 | 0.15, 0.52 | 6 | 0.30 | 0.15, 0.52 | 0.00 | −0.13, |
| | | (0.69, 1.86) | | | | | | | | | 0.13 |
| | | 10¹² | 5 | 5 | 1.00 | 0.57, 1.00 | 5 | 1.00 | 0.57, 1.00 | 0.00 | −0.47, |
| | | (4.73, 10¹²) | | | | | | | | | 0.47 |
| Dried | Salmonella | N/A | 5 | 0 | 0.00 | 0.00, 0.43 | 0 | 0.00 | 0.00, 0.43 | 0.00 | −0.47, |
| Hemp | Typhimurium | | | | | | | | | | 0.47 |
| Flower | SAL54 | 1.19 | 20 | 14 | 0.70 | 0.48, 0.75 | 15 | 0.75 | 0.53, 0.89 | -0.05 | −0.21, |
| 24 h | | (0.73, 2.09) | | | | | | | | | 0.11 |
| | | 10¹² | 5 | 5 | 1.00 | 0.57, 1.00 | 5 | 1.00 | 0.57, 1.00 | 0.00 | −0.47, |
| | | (8.18, 10¹²) | | | | | | | | | 0.47 |
| Dried | Salmonella | N/A | 5 | 1 | 0.20 | 0.00, 0.62 | 0 | 0.00 | 0.00, 0.43 | 0.20 | −0.36, |
| Cannabis | Typhimurium | | | | | | | | | | 0.76 |
| Flower^{l} | ATCC^{m} | 1.74 | 20 | 15 | 0.75 | 0.53, 0.89 | 14 | 0.70 | 0.48, 0.86 | 0.05 | −0.11, |
| 24 h | 14028 | (0.91, 8.08) | | | | | | | | | 0.21 |
| | | 4.90 | 5 | 5 | 1.00 | 0.57, 1.00 | 5 | 1.00 | 0.57, 1.00 | 0.00 | −0.47, |
| | | (2.50, 16.2) | | | | | | | | | 0.47 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes by the total number of trials.
^{e}Confirmed results = Leafy greens confirmed per BAM Ch. 5, dried cannabis flowers confirmed per SMPR 2020.002.
^{f}POD_{CC} = Candidate method confirmed positive outcomes divided by the total number of trials.
^{g}dPOD_{CP} = Difference between the candidate method presumptive result and candidate method confirmed result POD values.
^{h}95% CI = If the confidence interval of a dPOD does not contain zero, then the difference is statistically significant at hte 5% level.
ⁱGFP Strain derived from FDA SAL5694, Food and Drug Administration, Silver Spring, MD.
^{j}N/A = Not applicable.
^{k}All test portions in the MPN were positive, leading to the high value.
^{l}Cannabis flower was tested by the independent laboratory. Result indicated final interpretation.
^{m}ATCC = American Type Culture Collections, Manassas, VA.

TABLE 33
SIMUL-qPCR Salmonella & STEC with GFP Assay vs. Reference Method
Salmonella Results - Individual lysates for leafy greens

| | | | | SIMUL-qPCR Salmonella & | | | | | | | |
|---|---|---|---|---|---|---|---|---|---|---|---|
| | | MPN^{a}/Test | | STEC with GFP Confirmed | | | BAM Ch. 5 | | | | |
| Matrix | Strain | portion | N^{b} | x^{c} | POD_{C}^{d} | 95% CI | x | POD_{R}^{e} | 95% CI | dPOD_{C}^{f} | 95% CI^{g} |
| Leafy | Salmonella | N/Aⁱ | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 10 h | SAL54^{h} | 1.097 | 20 | 6 | 0.30 | 0.15, 0.52 | 6 | 0.30 | 0.15, 0.52 | 0.00 | −0.27, |
| | | (0.69, 1.86) | | | | | | | | | 0.27 |
| | | 10¹² | 5 | 5 | 1.00 | 0.57, 1.00 | 5 | 1.00 | 0.57, 1.00 | 0.00 | −0.43, |
| | | (4.73, 10¹²)^{j} | | | | | | | | | 0.43 |
| Leafy | Salmonella | N/A | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 18 h | SAL54 | 1.097 | 20 | 6 | 0.30 | 0.15, 0.52 | 6 | 0.30 | 0.15, 0.52 | 0.00 | −0.27, |
| | | (0.69, 1.86) | | | | | | | | | 0.27 |
| | | 10¹² | 5 | 5 | 1.00 | 0.57, 1.00 | 5 | 1.00 | 0.57, 1.00 | 0.00 | −0.43, |
| | | (4.73, 10¹²) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}GFP Strain derived from FDA SAL5694, Food and Drug Administration, Silver Spring, MD.
ⁱN/A = Not applicable.
^{j}All test portions in the MPN were positive, leading to the high value.

TABLE 34
SIMUL-qPCR Salmonella & STEC with GFP Assay Presumptive vs. Confirmed Salmonella
Results - Pooled lysates for leafy greens, dried hemp flower, and dried cannabis flower

| | | | | SIMUL-qPCR Salmonella & | | | SIMUL-qPCR Salmonella | | | | |
|---|---|---|---|---|---|---|---|---|---|---|---|
| | | | | STEC with GFP | | | & STEC with GFP | | | | |
| | | MPN^{a}/Test | | Presumptive | | | Confirmed^{e} | | | | |
| Matrix | Strain | portion | N^{b} | x^{c} | POD_{CP}^{d} | 95% CI | x | POD_{CC}^{f} | 95% CI | dPOD_{CP}^{g} | 95% CI^{h} |
| Leafy | Salmonella | N/A^{j} | 5 | 0 | 0.00 | 0.00, 0.43 | 0 | 0.00 | 0.00, 0.43 | 0.00 | −0.47, |
| Greens | Typhimurium | | | | | | | | | | 0.47 |
| 12 h | SAL54ⁱ | 1.097 | 20 | 6 | 0.30 | 0.15, 0.52 | 6 | 0.30 | 0.15, 0.52 | 0.00 | −0.13, |
| | | (0.69, 1.86) | | | | | | | | | 0.13 |
| | | 101² | 5 | 5 | 1.00 | 0.57, 1.00 | 5 | 1.00 | 0.57, 1.00 | 0.00 | −0.47, |
| | | (4.73, 101²)^{k} | | | | | | | | | 0.47 |
| Leafy | Salmonella | N/A | 5 | 0 | 0.00 | 0.00, 0.43 | 0 | 0.00 | 0.00, 0.43 | 0.00 | −0.47, |
| Greens | Typhimurium | | | | | | | | | | 0.47 |
| 18 h | SAL54 | 1.097 | 20 | 6 | 0.30 | 0.15, 0.52 | 6 | 0.30 | 0.15, 0.52 | 0.00 | −0.13, |
| | | (0.69, 1.86) | | | | | | | | | 0.13 |
| | | 10¹² | 5 | 5 | 1.00 | 0.57, 1.00 | 5 | 1.00 | 0.57, 1.00 | 0.00 | −0.47, |
| | | (4.73, 10¹²) | | | | | | | | | 0.47 |
| Dried | Salmonella | N/A | 5 | 0 | 0.00 | 0.00, 0.43 | 0 | 0.00 | 0.00, 0.43 | 0.00 | −0.47, |
| Hemp | Typhimurium | | | | | | | | | | 0.47 |
| Flower | SAL54 | 1.19 | 20 | 15 | 0.75 | 0.53, 0.89 | 15 | 0.75 | 0.53, 0.89 | 0.00 | −0.13, |
| 24 h | | (0.73, 2.09) | | | | | | | | | 0.13 |
| | | 10¹² | 5 | 5 | 1.00 | 0.57, 1.00 | 5 | 1.00 | 0.57, 1.00 | 0.00 | −0.47, |
| | | (8.18, 10¹²) | | | | | | | | | 0.47 |
| Dried | Salmonella | N/A | 5 | 0 | 0.00 | 0.00, 0.43 | 0 | 0.00 | 0.00, 0.43 | 0.00 | −0.47, |
| Cannabis | Typhimurium | | | | | | | | | | 0.47 |
| Flower^{l} | ATCC | 1.74 | 20 | 14 | 0.70 | 0.48, 0.86 | 14 | 0.70 | 0.48, 0.86 | 0.00 | −0.13, |
| 24 h | 14028 | (0.91, 8.08) | | | | | | | | | 0.13 |
| | | 4.90 | 5 | 5 | 1.00 | 0.57, 1.00 | 5 | 1.00 | 0.57, 1.00 | 0.00 | −0.47, |
| | | (2.50, 16.2) | | | | | | | | | 0.47 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes by the total number of trials.
^{e}Confirmed results = Leafy greens confirmed per BAM Ch. 5, dried cannabis flowers confirmed per SMPR 2020.002.
^{f}POD_{CC} = Candidate method confirmed positive outcomes divided by the total number of trials.
^{g}dPOD_{CP} = Difference between the candidate method presumptive result and candidate method confirmed result POD values.
^{h}95% CI = If the confidence interval of a dPOD does not contain zero, then the difference is statistically significant at hte 5% level.
ⁱGFP Strain derived from FDA SAL5694, Food and Drug Administration, Silver Spring, MD.
^{j}N/A = Not applicable.
^{k}All test portions in the MPN were positive, leading to the high value.
^{l}Cannabis flower was tested by the independent laboratory. Result indicated final interpretation.
^{m}ATCC = American Type Culture Collections, Manassas, VA.

TABLE 35
SIMUL-qPCR Salmonella & STEC with GFP Assay vs. Reference Method
Salmonella Results - Pooled lysates for leafy greens

| | | | | SIMUL-qPCR Salmonella & | | | | | | | |
|---|---|---|---|---|---|---|---|---|---|---|---|
| | | MPN^{a}/Test | | STEC with GFP Confirmed | | | BAM Ch. 5 | | | | |
| Matrix | Strain | portion | N^{b} | x^{c} | POD_{C}^{d} | 95% CI | x | POD_{R}^{e} | 95% CI | dPOD_{C}^{f} | 95% CI^{g} |
| Leafy | Salmonella | N/Aⁱ | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 10 h | SAL54^{h} | 1.097 | 20 | 6 | 0.30 | 0.15, 0.52 | 6 | 0.30 | 0.15, 0.52 | 0.00 | −0.27, |
| | | (0.69, 1.86) | | | | | | | | | 0.27 |
| | | 10¹² | 5 | 5 | 1.00 | 0.57, 1.00 | 5 | 1.00 | 0.57, 1.00 | 0.00 | −0.43, |
| | | (4.73, 10¹²)^{j} | | | | | | | | | 0.43 |
| Leafy | Salmonella | N/A | 5 | 0 | 0.00 | 0.00, 0.43 | 0 | 0.00 | 0.00, 0.43 | 0.00 | −0.43, |
| Greens | Typhimurium | | | | | | | | | | 0.43 |
| 18 h | SAL54 | 1.097 | 20 | 6 | 0.30 | 0.15, 0.52 | 6 | 0.30 | 0.15, 0.52 | 0.00 | −0.27, |
| | | (0.69, 1.86) | | | | | | | | | 0.27 |
| | | 10¹² | 5 | 5 | 1.00 | 0.57, 1.00 | 5 | 1.00 | 0.57, 1.00 | 0.00 | −0.43, |
| | | (4.73, 10¹²) | | | | | | | | | 0.43 |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}GFP Strain derived from FDA SAL5694, Food and Drug Administration, Silver Spring, MD.
ⁱN/A = Not applicable.
^{j}All test portions in the MPN were positive, leading to the high value.

TABLE 36
SIMUL-qPCR Salmonella & STEC with GFP Assay Presumptive vs. Confirmed STEC Results
- Individual lysates for leafy greens, dried hemp flower, and dried cannabis flower

| | | | | SIMUL-qPCR Salmonella | | | SIMUL-qPCR Salmonella | | | | |
|---|---|---|---|---|---|---|---|---|---|---|---|
| | | | | & STEC with GFP | | | & STEC with GFP | | | | |
| | | MPN^{a}/Test | | Presumptive | | | Confirmede | | | | |
| Matrix | Strain | portion | N^{b} | x^{c} | POD_{CP}^{d} | 95% CI | x | POD_{CC}^{f} | 95% CI | dPOD_{CP}^{g} | 95% CI^{h} |
| Leafy | E. coli | N/A^{j} | 5 | 0 | 0.00 | 0.00, 0.43 | 0 | 0.00 | 0.00, 0.43 | 0.00 | −0.47, 0.47 |
| Greens | O157:H7 | 1.097 | 20 | 13 | 0.65 | 0.43, 0.82 | 13 | 0.65 | 0.43, 0.82 | 0.00 | −0.13, 0.13 |
| 10 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | −0.47, 0.47 |
| | FDAⁱ | (4.73, 10¹²)^{k} | | | | | | | | | |
| | ESC1177 | | | | | | | | | | |
| Leafy | E. coli | N/A | 5 | 0 | 0.00 | 0.00, 0.43 | 0 | 0.00 | 0.00, 0.43 | 0.00 | −0.47, 0.47 |
| Greens | O157:H7 | 1.097 | 20 | 13 | 0.65 | 0.43, 0.82 | 13 | 0.65 | 0.43, 0.82 | 0.00 | −0.13, 0.13 |
| 18 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | −0.47, 0.47 |
| | FDA | (4.73, 10¹²) | | | | | | | | | |
| | ESC1177 | | | | | | | | | | |
| Dried | E. coli | N/A | 5 | 0 | 0.00 | 0.00, 0.43 | 0 | 0.00 | 0.00, 0.43 | 0.00 | −0.47, 0.47 |
| Hemp | O157:H7 | 1.33 | 20 | 13 | 0.65 | 0.43, 0.82 | 13 | 0.65 | 0.43, 0.82 | 0.00 | −0.13, 0.13 |
| Flower | EC 43 | (0.84, 2.18) | | | | | | | | | |
| 24 h | derived from | 10¹² | 5 | 5 | 1.00 | 0.57, 1.00 | 5 | 1.00 | 0.57, 1.00 | 0.00 | −0.47, 0.47 |
| | FDA | (8.18, 10¹²) | | | | | | | | | |
| | ESC1177 | | | | | | | | | | |
| Dried | E. coli | N/A | 5 | 1 | 0.20 | 0.00, 0.62 | 0 | 0.00 | 0.00, 0.43 | 0.20 | −0.36, 0.76 |
| Cannabis | O157:H7 | 0.88 | 20 | 10 | 0.50 | 0.29, 0.70 | 10 | 0.50 | 0.29, 0.70 | 0.00 | −0.13, 0.13 |
| Flower^{l} | ATCC^{m} | (0.40, 2.02) | | | | | | | | | |
| 24 h | 43895 | 2.96 | | | | | | | | | |
| | | (1.54, 9.78) | | | | | | | | | |

^{a}MPN = Most Probable Number is based on the POD of reference method test portions using the LVF MPN calculator, with 95% confidence interval.
^{b}N = Number of test potions.
^{c}x = Number of positive test portions.
^{d}POD_{CP} = Candidate method presumptive positive outcomes divided by the total number of trials.
^{e}Confirmed results = Leafy greens confirmed per BAM Ch. 4A, dried hemp and dried cannabis flowers confirmed per SMPR 2020.012.
^{f}POD_{CC} = Candidate method confirmed positive outcomes divided by the total number of trials.
^{g}dPOD_{CP} = Difference between the candidate method presumptive result and candidate method confirmed result POD values.
^{h}95% CI = If the confidence interval of a dPOD does not contain zero, then the difference is statistically significant at the 5% level.
ⁱGFP Strain derived from FDA ESC117, Food and Drug Administration, Silver Spring, MD.
^{j}N/A = Not applicable.
^{k}All test portions in the MPN were positive, leading to the high value.
^{l}Cannabis flower was tested by the independent laboratory. Result indicates final interpretation.
^{m}ATCC = American Type Culture Collection, Manassas, VA.

TABLE 37
SIMUL-qPCR Salmonella & STEC with GFP Assay vs. Reference
Method STEC Results - Individual lysates for leafy greens

| | | | | SIMUL-qPCR Salmonella & | | | | | | | |
|---|---|---|---|---|---|---|---|---|---|---|---|
| | | MPN^{a}/Test | | STEC with GFP Confirmed | | | BAM Ch. 4A | | | | |
| Matrix | Strain | portion | N^{b} | x^{c} | POD_{C}^{d} | 95% CI | x | POD_{R}^{e} | 95% CI | dPOD_{C}^{f} | 95% CI^{g} |
| Leafy | E. coli | N/Aⁱ | 5 | 0 | 0.00 | 0.00, 0.43 | 0 | 0.00 | 0.00, 0.43 | 0.00 | -0.43, 0.43 |
| Greens | O157:H7 | 1.097 | 20 | 13 | 0.65 | 0.43, 0.82 | 14 | 0.70 | 0.48, 0.85 | -0.05 | -0.32, 0.23 |
| 10 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | -0.43, 0.43 |
| | FDA^{h} | (4.73, 10¹²)^{j} | | | | | | | | | |
| | ESC1177 | | | | | | | | | | |
| Leafy | E. coli | N/A | 5 | 0 | 0.00 | 0.00, 0.43 | 0 | 0.00 | 0.00, 0.43 | 0.00 | -0.43, 0.43 |
| Greens | O157:H7 | 1.097 | 20 | 13 | 0.65 | 0.43, 0.82 | 14 | 0.70 | 0.48, 0.85 | -0.05 | -0.32, 0.23 |
| 18 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | -0.43, 0.43 |
| | FDA | (4.73, 10¹²) | | | | | | | | | |
| | ESC1177 | | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}GFP Strain derived from FDA ESC1177, Food and Drug Administration, Silver Spring, MD.
ⁱN/A = Not applicable.
^{j}All test portions in the MPN were positive, leading to the high value.

TABLE 38
SIMUL-qPCR Salmonella & STEC with GFP Assay Presumptive vs. Confirmed STEC Results
- Pooled lysates for leafy greens, dried hemp flower, and cannabis flower

| | | | | SIMUL-qPCR Salmonella & | | | SIMUL- Salmonella & | | | | |
|---|---|---|---|---|---|---|---|---|---|---|---|
| | | | | STEC with GFP | | | STEC with GFP | | | | |
| | | MPN^{a}/Test | | Presumptive | | | Confirmed^{e} | | | | |
| Matrix | Strain | portion | N^{b} | x^{c} | POD_{CP}^{d} | 95% CI | X | POD_{CC}^{f} | 95% CI | dPOD_{CP}^{g} | 95% CI^{h} |
| Leafy | E. coli | N/A^{j} | 5 | 0 | 0.00 | 0.00, 0.43 | 0 | 0.00 | 0.00, 0.43 | 0.00 | −0.47, 0.47 |
| Greens | O157:H7 | 1.097 | 20 | 13 | 0.65 | 0.43, 0.82 | 13 | 0.65 | 0.43, 0.82 | 0.00 | −0.13, 0.13 |
| 12 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | −0.47, 0.47 |
| | FDAⁱ | (4.73, 10¹²)^{k} | | | | | | | | | |
| | ESC1177 | | | | | | | | | | |
| Leafy | E. coli | N/Aⁱ | 5 | 0 | 0.00 | 0.00, 0.43 | 0 | 0.00 | 0.00, 0.43 | 0.00 | −0.47, 0.47 |
| Greens | O157:H7 | 1.097 | 20 | 15 | 0.75 | 0.53, 0.89 | 13 | 0.65 | 0.43, 0.82 | 0.00 | −0.08, 0.28 |
| 18 h | EC 43 | (0.69, 1.86) | | | | | | | | | |
| | derived from | 10¹² | 5 | 5 | 1.00 | 0.57, 1.00 | 5 | 1.00 | 0.57, 1.00 | 0.00 | −0.47, 0.47 |
| | FDA^{h} | (4.73, 10¹²)^{j} | | | | | | | | | |
| | ESC1177 | | | | | | | | | | |
| Dried | E. coli | N/A | 5 | 0 | 0.00 | 0.00, 0.43 | 0 | 0.00 | 0.00, 0.43 | 0.00 | −0.47, 0.47 |
| Hemp | O157:H7 | 1.33 | 20 | 12 | 0.60 | 0.39, 0.78 | 13 | 0.65 | 0.43, 0.82 | −0.05 | −0.21, 0.11 |
| Flower | EC 43 | (0.84, 2.18) | | | | | | | | | |
| 24 h | derived from | 10¹² | 5 | 5 | 1.00 | 0.57, 1.00 | 5 | 1.00 | 0.57, 1.00 | 0.00 | −0.47, 0.47 |
| | FDA | (8.18, 10¹²) | | | | | | | | | |
| | ESC1177 | | | | | | | | | | |
| Dried | E. coli | N/A | 5 | 0 | 0.00 | 0.00, 0.43 | 0 | 0.00 | 0.00, 0.43 | 0.20 | −0.47, 0.47 |
| Cannabis | O157:H7 | 0.88 | 20 | 10 | 0.50 | 0.29, 0.70 | 10 | 0.50 | 0.29, 0.70 | 0.00 | −0.13, 0.13 |
| Flower^{l} | ATCC^{m} | (0.40, 2.02) | | | | | | | | | |
| 24 h | 43895 | 2.96 | 5 | 5 | 1.00 | 0.57, 1.00 | 5 | 1.00 | 0.57, 1.00 | 0.00 | −0.47, 0.47 |
| | | (1.54, 9.78) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{CP} = Candidate method presumptive positive outcomes divided by the total number of trials.
Confirmed results = Leafy greens confirmed per BAM Ch. 4A, dried hemp and dried cannabis flowers confirmed per SMPR 2020.012.
^{f}POD_{CC} = Candidate method confirmed positive outcomes divided by the total number of trials.
^{g}dPOD_{CP} = Difference between the candidate method presumptive results and candidate method confirmed result POD values.
^{h}95% CI = If the confidence interval of a dPOD does not contain zero, then the difference is statistically significant at the 5 % level.
ⁱGFP Strain derived from FDA ESC1177, Food and Drug Administration, Silver Spring, MD.
^{j}N/A = Not applicable.
^{k}All test portions in the MPN were positive, leading to the high value.
^{l}Cannabis flower was tested by the independent laboratory. Lysates were shipped to AFD, where the pooled testing was completed.
^{m}ATCC = American Type Culture Collection, Manassas, VA.

TABLE 39
SIMUL-qPCR Salmonella & STEC with GFP Assay vs. Reference
Method STEC Results - Pooled lysates for leafy greens

| | | | | SIMUL-qPCR Salmonella & | | | | | | | |
|---|---|---|---|---|---|---|---|---|---|---|---|
| | | MPN^{a}/Test | | STEC with GFP Confirmed | | | BAM Ch. 4A | | | | |
| Matrix | Strain | portion | N^{b} | x^{c} | POD_{C}d | 95% CI | x | POD_{R}^{e} | 95% CI | dPOD_{C}^{f} | 95% CI^{g} |
| Leafy | E. coli | N/Aⁱ | 5 | 0 | 0.00 | 0.00, 0.43 | 0 | 0.00 | 0.00, 0.43 | 0.00 | −0.43, 0.43 |
| Greens | O157:H7 | 1.097 | 20 | 13 | 0.65 | 0.43, 0.82 | 14 | 0.70 | 0.48, 0.85 | −0.05 | −0.32, 0.23 |
| 12 h | EC 43 derived | (0.69, 1.86) | | | | | | | | | |
| | from FDA^{h} | 10¹² | 5 | 5 | 1.00 | 0.57, 1.00 | 5 | 1.00 | 0.57, 1.00 | 0.00 | −0.43, 0.43 |
| | ESC1177 | (4.73, 10¹²)^{j} | | | | | | | | | |
| Leafy | E. coli | N/A | 5 | 0 | 0.00 | 0.00, 0.43 | 0 | 0.00 | 0.00, 0.43 | 0.00 | −0.43, 0.43 |
| Greens | O157:H7 | 1.097 | 20 | 13 | 0.65 | 0.43, 0.82 | 14 | 0.70 | 0.48, 0.85 | −0.05 | −0.32, 0.23 |
| 18 h | EC 43 derived | (0.69, 1.86) | | | | | | | | | |
| | from FDA^{h} | 10¹² | 5 | 5 | 1.00 | 0.57, 1.00 | 5 | 1.00 | 0.57, 1.00 | 0.00 | −0.43, 0.43 |
| | ESC1177 | (4.73, 10¹²) | | | | | | | | | |

^{a}MPN = Most Probable Number is based on the POD of reference method test portions using the LCF MPN calculator, with 95% confidence interval.
^{b}N = Number of test potions.
^{c}x = Number of positive test portions.
^{d}POD_{C} = Candidate method presumptive positive outcomes confirmed positive divided by the total number of trials.
^{e}POD_{R} = Reference method positive outcomes divided by the total number of trials.
^{f}dPOD_{C} = Difference between the candidate method result and reference method result POD values.
^{g}95% CI = If the confidence interval of a dPOD does not contain zero, then the difference is statistically significant at the 5% level.
^{h}GFP Strain derived from FDA ESC1177, Food and Drug Administration, Silver Spring, MD.
ⁱN/A = Not applicable.
^{j}All test portions in the MPN were positive, leading to the high value.

TABLE 40
Robustness variations vs. SIMUL-qPCR Salmonella & STEC with GFP Assay - Leafy greens

| | | SIMUL-qPCR Salmonella & | | | SIMUL-qPCR Salmonella & | | | | |
|---|---|---|---|---|---|---|---|---|---|
| | | STEC with GFP Test | | | STEC with GFP Recommended | | | | |
| | | Conditions | | | Conditions | | | | |
| Test Conditions^{a} | N^{b} | x^{c} | POD_{TC}^{d} | 95% CI | x | POD_{R}^{e} | 95% CI | dPOD_{TC}^{f} | 95% CI^{g} |

Leafy Greens, S. Typhimurium (FDA^{h} SAL54) results

| | | | | | | | | | |
|---|---|---|---|---|---|---|---|---|---|
| 9 h, 380 μL, 15 μL | 10 | 6 | 0.60 | 0.31, 0.83 | 6 | 0.60 | 0.31, 0.83 | 0.00 | −0.37, 0.37 |
| 9 h, 380 μL, 25 μL | 10 | 6 | 0.60 | 0.31, 0.83 | 6 | 0.60 | 0.31, 0.83 | 0.00 | −0.37, 0.37 |
| 9 h, 420 μL, 15 μL | 10 | 6 | 0.60 | 0.31, 0.83 | 6 | 0.60 | 0.31, 0.83 | 0.00 | −0.37, 0.37 |
| 9 h, 420 μL, 25 μL | 10 | 6 | 0.60 | 0.31, 0.83 | 6 | 0.60 | 0.31, 0.83 | 0.00 | −0.37, 0.37 |
| 24 h, 380 μL, 15 μL | 10 | 6 | 0.60 | 0.31, 0.83 | 6 | 0.60 | 0.31, 0.83 | 0.00 | −0.37, 0.37 |
| 24 h, 380 μL, 25 μL | 10 | 7 | 0.70 | 0.40, 0.89 | 6 | 0.60 | 0.31, 0.83 | 0.10 | −0.28, 0.45 |
| 24 h, 420 μL, 15 μL | 10 | 6 | 0.60 | 0.31, 0.83 | 6 | 0.60 | 0.31, 0.83 | 0.00 | −0.37, 0.37 |
| 24 h, 420 μL, 25 μL | 10 | 7 | 0.70 | 0.40, 0.89 | 6 | 0.60 | 0.31, 0.83 | 0.10 | −0.28, 0.45 |

Leafy Greens, E. coli O157:H7 (FDA EC43) results

| | | | | | | | | | |
|---|---|---|---|---|---|---|---|---|---|
| 9 h, 380 μL, 15 μL | 10 | 7 | 0.70 | 0.40, 0.89 | 7 | 0.70 | 0.40, 0.89 | 0.00 | −0.36, 0.36 |
| 9 h, 380 μL, 25 μL | 10 | 7 | 0.70 | 0.40, 0.89 | 7 | 0.70 | 0.40, 0.89 | 0.00 | −0.36, 0.36 |
| 9 h, 420 μL, 15 μL | 10 | 7 | 0.70 | 0.40, 0.89 | 7 | 0.70 | 0.40, 0.89 | 0.00 | −0.36, 0.36 |
| 9 h, 420 μL, 25 μL | 10 | 7 | 0.70 | 0.40, 0.89 | 7 | 0.70 | 0.40, 0.89 | 0.00 | −0.36, 0.36 |
| 24 h, 380 μL, 15 μL | 10 | 7 | 0.70 | 0.40, 0.89 | 7 | 0.70 | 0.40, 0.89 | 0.00 | −0.36, 0.36 |
| 24 h, 380 μL, 25 μL | 10 | 7 | 0.70 | 0.40, 0.89 | 7 | 0.70 | 0.40, 0.89 | 0.00 | −0.36, 0.36 |
| 24 h, 420 μL, 15 μL | 10 | 7 | 0.70 | 0.40, 0.89 | 7 | 0.70 | 0.40, 0.89 | 0.00 | −0.36, 0.36 |
| 24 h, 420 μL, 25 μL | 10 | 7 | 0.70 | 0.40, 0.89 | 7 | 0.70 | 0.40, 0.89 | 0.00 | −0.36, 0.36 |

Leafy Greens, uncontaminated

| | | | | | | | | | |
|---|---|---|---|---|---|---|---|---|---|
| 9 h, 380 μL, 15 μL | 10 | 0 | 0.00 | 0.00, 0.28 | 0 | 0.00 | 0.00, 0.28 | 0.00 | −0.28, 0.28 |
| 9 h, 380 μL, 25 μL | 10 | 0 | 0.00 | 0.00, 0.28 | 0 | 0.00 | 0.00, 0.28 | 0.00 | −0.28, 0.28 |
| 9 h, 420 μL, 15 μL | 10 | 0 | 0.00 | 0.00, 0.28 | 0 | 0.00 | 0.00, 0.28 | 0.00 | −0.28, 0.28 |
| 9 h, 420 μL, 25 μL | 10 | 0 | 0.00 | 0.00, 0.28 | 0 | 0.00 | 0.00, 0.28 | 0.00 | −0.28, 0.28 |
| 24 h, 380 μL, 15 μL | 10 | 0 | 0.00 | 0.00, 0.28 | 0 | 0.00 | 0.00, 0.28 | 0.00 | −0.28, 0.28 |
| 24 h, 380 μL, 25 L | 10 | 0 | 0.00 | 0.00, 0.28 | 0 | 0.00 | 0.00, 0.28 | 0.00 | −0.28, 0.28 |
| 24 h, 420 μL, 15 μL | 10 | 0 | 0.00 | 0.00, 0.28 | 0 | 0.00 | 0.00, 0.28 | 0.00 | −0.28, 0.28 |
| 24 h, 420 μL, 25 μL | 10 | 0 | 0.00 | 0.00, 0.28 | 0 | 0.00 | 0.00, 0.28 | 0.00 | −0.28, 0.28 |

^{a}Test conditions: Enrichment time, volume of extraction reagent, volume of extracted DNA sample. Recommended condition; 24 h enrichment, 995 μL extraction reagent, 20 μL extracted sample.
^{b}N = Number of test potions.
^{c}x = Number of positive test portions.
^{d}POD_{TC} = Test condition combination positive outcomes divided by the total number of trials.
^{e}POD_{R} = Recommended test condition positive outcomes divided by the total number of trials.
^{f}dPOD_{TC} = Difference between the test condition combination and the recommended test condition POD values.
^{g}95% CI = If the confidence interval of a dPOD does not contain zero, then the difference is statistically significant at the 5% level.
^{h}GFP Strain derived from FDA SAL54, Food and Drug Administration, Silver Spring, MD.

TABLE 41
Robustness Variations vs. SIMUL-qPCR Salmonella & STEC with GFP Assay - Hemp flower (≤0.3%
THC), artificially contaminated with S. Typhimurium (FDA^{h} SAL54) and E. coli O157:H7 (FDA EC43)

| | | SIMUL-qPCR Salmonella & | | | SIMUL-qPCR Salmonella & | | | | |
|---|---|---|---|---|---|---|---|---|---|
| | | STEC with GFP Test | | | STEC with GFP Recommended | | | | |
| | | Conditions | | | Conditions | | | | |
| Test Conditions^{a} | N^{b} | x^{c} | POD_{TC}^{d} | 95% CI | x | POD_{R}^{e} | 95% CI | dPOD_{TC}^{f} | 95% CI^{g} |

Dried Hemp Flower (≤0.3% THC), S. Typhimurium (FDAh SAL54) results

| | | | | | | | | | |
|---|---|---|---|---|---|---|---|---|---|
| 20 h, 975 μL, 15 μL | 10 | 7 | 0.70 | 0.40, 0.89 | 6 | 0.60 | 0.31, 0.83 | 0.10 | −0.28, 0.45 |
| 20 h, 975 μL, 25 μL | 10 | 7 | 0.70 | 0.40, 0.89 | 6 | 0.60 | 0.31, 0.83 | 0.10 | −0.28, 0.45 |
| 20 h, 1015 μL, 15 μL | 10 | 7 | 0.70 | 0.40, 0.89 | 6 | 0.60 | 0.31, 0.83 | 0.10 | −0.28, 0.45 |
| 20 h, 1015 μL, 25 μL | 10 | 7 | 0.70 | 0.40, 0.89 | 6 | 0.60 | 0.31, 0.83 | 0.10 | −0.28, 0.45 |
| 30 h, 975 μL, 15 μL | 10 | 7 | 0.70 | 0.40, 0.89 | 6 | 0.60 | 0.31, 0.83 | 0.10 | −0.28, 0.45 |
| 30 h, 975 μL, 25 μL | 10 | 7 | 0.70 | 0.40, 0.89 | 6 | 0.60 | 0.31, 0.83 | 0.10 | −0.28, 0.45 |
| 30 h, 1015 μL, 15 μL | 10 | 7 | 0.70 | 0.40, 0.89 | 6 | 0.60 | 0.31, 0.83 | 0.10 | −0.28, 0.45 |
| 30 h, 1015 μL, 25 μL | 10 | 7 | 0.70 | 0.40, 0.89 | 6 | 0.60 | 0.31, 0.83 | 0.10 | −0.28, 0.45 |

Dried Hemp Flower (≤0.3% THC), E. coli O157:H7 (FDA EC43) results

| | | | | | | | | | |
|---|---|---|---|---|---|---|---|---|---|
| 20 h, 975 μL, 15 μL | 10 | 6 | 0.60 | 0.31, 0.83 | 6 | 0.60 | 0.31, 0.83 | 0.00 | −0.37, 0.37 |
| 20 h, 975 μL, 25 μL | 10 | 6 | 0.60 | 0.31, 0.83 | 6 | 0.60 | 0.31, 0.83 | 0.00 | −0.37, 0.37 |
| 20 h, 1015 μL, 15 μL | 10 | 6 | 0.60 | 0.31, 0.83 | 6 | 0.60 | 0.31, 0.83 | 0.00 | −0.37, 0.37 |
| 20 h, 1015 μL, 25 μL | 10 | 6 | 0.60 | 0.31, 0.83 | 6 | 0.60 | 0.31, 0.83 | 0.00 | −0.37, 0.37 |
| 30 h, 975 μL, 15 μL | 10 | 6 | 0.60 | 0.31, 0.83 | 6 | 0.60 | 0.31, 0.83 | 0.00 | −0.37, 0.37 |
| 30 h, 975 μL, 25 μL | 10 | 6 | 0.60 | 0.31, 0.83 | 6 | 0.60 | 0.31, 0.83 | 0.00 | −0.37, 0.37 |
| 30 h, 1015 μL, 15 μL | 10 | 6 | 0.60 | 0.31, 0.83 | 6 | 0.60 | 0.31, 0.83 | 0.00 | −0.37, 0.37 |
| 30 h, 1015 μL, 25 μL | 10 | 6 | 0.60 | 0.31, 0.83 | 6 | 0.60 | 0.31, 0.83 | 0.00 | −0.37, 0.37 |

Dried Hemp Flower (≤0.3% THC), uncontaminated

| | | | | | | | | | |
|---|---|---|---|---|---|---|---|---|---|
| 20 h, 975 μL, 15 μL | 10 | 0 | 0.00 | 0.00, 0.28 | 0 | 0.00 | 0.00, 0.28 | 0.00 | −0.28, 0.28 |
| 20 h, 975 μL, 25 μL | 10 | 0 | 0.00 | 0.00, 0.28 | 0 | 0.00 | 0.00, 0.28 | 0.00 | −0.28, 0.28 |
| 20 h, 1015 μL, 15 μL | 10 | 0 | 0.00 | 0.00, 0.28 | 0 | 0.00 | 0.00, 0.28 | 0.00 | −0.28, 0.28 |
| 20 h, 1015 μL, 25 μL | 10 | 0 | 0.00 | 0.00, 0.28 | 0 | 0.00 | 0.00, 0.28 | 0.00 | −0.28, 0.28 |
| 30 h, 975 μL, 15 μL | 10 | 0 | 0.00 | 0.00, 0.28 | 0 | 0.00 | 0.00, 0.28 | 0.00 | −0.28, 0.28 |
| 30 h, 975 μL, 25 μL | 10 | 0 | 0.00 | 0.00, 0.28 | 0 | 0.00 | 0.00, 0.28 | 0.00 | −0.28, 0.28 |
| 30 h, 1015 μL, 15 μL | 10 | 0 | 0.00 | 0.00, 0.28 | 0 | 0.00 | 0.00, 0.28 | 0.00 | −0.28, 0.28 |
| 30 h, 1015 μL, 25 μL | 10 | 0 | 0.00 | 0.00, 0.28 | 0 | 0.00 | 0.00, 0.28 | 0.00 | −0.28, 0.28 |

^{a}Test conditions: Enrichment time, volume of extraction reagent, volume of extracted DNA sample. Recommended condition; 24 h enrichment, 995 μL extraction reagent, 20 μL extracted sample.
^{b}N = Number of test potions.
^{c}x = Number of positive test portions.
^{d}POD_{TC} = Test condition combination positive outcomes divided by the total number of trials.
^{e}POD_{R} = Recommended test condition positive outcomes divided by the total number of trials.
^{f}dPOD_{TC}= Difference between the test condition combination and the recommended test condition POD values.
^{g}95% CI = If the confidence interval of a dPOD does not contain zero, then the difference is statistically significant at the 5% level.
^{h}GFP Strain derived from FDA SAL54, Food and Drug Administration, Silver Spring, MD.

TABLE 42
SIMUL-qPCR Salmonella & STEC with GFP Assay Stability Results: 0 Month vs. 6 Month

| | | SIMUL-qPCR Salmonella & STEC | | | SIMUL-qPCR Salmonella & | | | | |
|---|---|---|---|---|---|---|---|---|---|
| | | with GFP | | | STEC with GFP | | | | |
| | | 0 Month Kit | | | 6 Month Kit | | | | |
| Strain | N^{a} | X^{b} | POD₀^{c} | 95% CI | x | POD₆^{d} | 95% CI | dPOD_{C}^{e} | 95% CI^{f} |
| S. Typhimurium | 10 | 7 | 0.70 | 0.40, 0.89 | 7 | 0.70 | 0.40, 0.89 | 0.00 | −0.36, 0.36 |
| (FDA^{g} SAL54) | | | | | | | | | |
| C. freundii (ATCC^{h} | 10 | 0 | 0.00 | 0.00, 0.28 | 0 | 0.00 | 0.00, 0.28 | 0.00 | −0.28, 0.28 |
| 43864) | | | | | | | | | |

^{a}N = Number of test potions.
^{b}x = Number of positive test portions.
^{c}POD₀ = Positive outcomes of the 0-month kit divided by the total number of trials.
^{d}POD₆ = Positive outcomes of the 6-month kit divided by the total number of trials.
^{e}dPOD_{C} = Difference between the 0-month kit and the 6-month kit POD values.
^{f}95% CI = If the confidence interval of a dPOD does not contain zero, then the difference is statistically significant at the 5% level.
^{g}GFP Strain derived from FDA SAL54, Food and Drug Administration, Silver Spring, MD.
^{h}ATCC = American Type Culture Collection, Manassas. VA.

TABLE 43
SIMUL-qPCR Salmonella & STEC with GFP Assay Stability Results: 0 Month vs. 12 Month

| | | SIMUL-qPCR Salmonella & STEC | | | SIMUL-qPCR Salmonella & | | | | |
|---|---|---|---|---|---|---|---|---|---|
| | | with GFP | | | STEC with GFP | | | | |
| | | 0 Month Kit | | | 12 Month Kit | | | | |
| Strain | N^{a} | X^{b} | POD₀^{c} | 95% CI | x | POD₁₂^{d} | 95% CI | dPOD_{C}^{e} | 95% CI^{f} |
| S. Typhimurium | 10 | 7 | 0.70 | 0.40, 0.89 | 7 | 0.70 | 0.40, 0.89 | 0.00 | −0.36, 0.36 |
| (FDA^{g} SAL54) | | | | | | | | | |
| C. freundii (ATCC^{h} | 10 | 0 | 0.00 | 0.00, 0.28 | 0 | 0.00 | 0.00, 0.28 | 0.00 | −0.28, 0.28 |
| 43864) | | | | | | | | | |

^{a}N = Number of test potions.
^{b}x = Number of positive test portions.
^{c}POD₀ = Positive outcomes of the 0-month kit divided by the total number of trials.
^{d}POD₁₂ = Positive outcomes of the 12-month kit divided by the total number of trials.
^{e}dPOD_{C} = Difference between the 0-month kit and the 12-month kit POD values.
^{f}95% CI = If the confidence interval of a dPOD does not contain zero, then the difference is statistically significant at the 5% level.
^{g}GFP Strain derived from FDA SAL54, Food and Drug Administration, Silver Spring, MD.
^{h}ATCC = American Type Culture Collection, Manassas. VA.

### Example 5-A Comparison of Cq Values Between E. coli 0157: H7 Target of Interest and the GFP Target

The Simultaneous Multiplex Real Time PCR (SIMUL-qPCR) E. coli O157:H7 with GFP Assay was tested as a quick and reliable method for detecting E. coli O157:H7 in manual sampling devices (“MSD”). As an improvement, a target to detect a Green Fluorescent Protein (GFP) alongside the E. coli O157:H7 and internal amplification control targets was tested and included.

Quality control strains tagged with a GFP allow technicians to determine if potential positives are due to cross-contamination from the internal strain. But in order to detect the GFP, potential positives needed to be streaked out on solid media for confirmation and then placed under the correct UV wavelength to look for any fluorescence. This step added a minimum of 24 hours to test results. By adding the GFP target directly to the SIMUL-qPCR assay, potential positives could be identified and verification that those potential positives were not caused by cross-contamination could be performed all in one PCR run.

A validation study on MSD was conducted using five different strains of E. coli O157:H7, one of which was tagged with a GFP. A total of one hundred MSD were divided into sets of twenty, and each set was inoculated with each of the five strains at near fractional levels (inoculum quantities ranged from 5-6 CFU/MSD). All samples were processed using the SIMUL-qPCR E. coli O157:H7 with GFP Assay procedure. All enrichments were processed for cultural confirmation regardless of the results obtained by the SIMUL-qPCR method to verify all results were accurate. Enrichments were streaked to modified rainbow agar, examined for typical morphology of E. coli O157:H7, and reviewed for green fluorescence under 315-400 nm UV light. All potential positive samples were expected to have typical colonies on rainbow agar. MSD inoculated with a strain of E. coli O157:H7 tagged with the GFP was anticipated to fluoresce on modified rainbow agar, while the ones inoculated without the GFP strain should not fluoresce on the agar.

To prepare the product materials for testing, one hundred manual sampling devices were used to swab one hundred different combo bins of beef trim. Twenty MSD were then spiked using E. coli O157:H7 containing GFP (FDA ESC1177), and the other eighty MSD were spiked with E. coli O157:H7 that did not contain GFP (twenty with ATCC 43886, twenty with NR-22047, twenty with ATCC 700728, and twenty with NCTC 12900). All strains had been grown from a slant stored at the 2-4° C. refrigerator and cultured in BHI at 37° C.±2° C. for 20-24 hours.

After incubation, the cultures were serially diluted in Butterfield's phosphate diluent to just above fractional levels. All MSD were inoculated between 5-6 CFU/MSD. Samples were hand massaged for two minutes to create homogenous mixtures before being placed in a refrigerator at 2-8° C. for 48 hours to allow for stabilization of the microorganisms in the food environment. Once stabilized, the samples were enriched in Buffered Peptone Water (BPW) pre-warmed to 45±1° C. and incubated for 10 hours. The temperature of the media was verified before adding it to the samples, ensuring that all samples were at the correct incubation temperature for the full 10 hours as media temperature will drop when added to the cold samples and can take a long time to get back up to the required incubation temperature. That incubation temperature was critical in not only growing the cells up to a level the method can detect in the time frame outlined, but also critical in the ability of the method to detect those low levels during the pooling process. After incubation, samples were tested three ways. Each MSD sample was lysed individually. Each sample was also composited prior to lysing by combining 20 μL of the inoculated sample with 80 μL of a known negative sample. Finally, each individual lysis was pooled by combining 5 μL of the lysis with 45 μL of a known negative lysis.

Results from the validation study of the SIMUL-qPCR E. coli O157:H7 with GFP Assay for the manual sampling devices are outlined in Tables 44-45. For all samples processed individually, through the wet compositing method, and through pooling of lysis, positive results were obtained in the E. coli O157:H7 target of the SIMUL-qPCR assay. Only the first twenty samples were positive for the GFP target on the PCR assay. All enrichments streaked to rainbow agar grew colonies that had typical morphology of E. coli O157:H7, but only the plates that were streaked from enrichments spiked with E. coli O157:H7 containing the GFP fluoresced. For each lysing method, the Cq values were evaluated after 10 hours of incubation. In almost all cases, the individual samples have the lowest Cq values, followed by the wet compositing, and then the pooled lysis. This is to be expected, as the wet composites were combined with known negative samples, which could create some inhibition, while the pooled lysis acts similarly to a dilution, which would cause a little bit of later Cq call. However, in some cases, the wet compositing and/or the pooled lysis led to an earlier Cq call. This could be due to the residue on the cloth from swabbing the combo bins or higher bacterial load causing some inhibition that was cleaned up when put into a dilution or negative sample enrichment. Regardless, none of these factors impacted the assay's ability of detecting either E. coli O157:H7 or the GFP on the manual sampling devices.

Data was evaluated using the empirical rule to show that all Cq values should fall within three standard deviations of the average Cq value. The empirical rule, also known as the three-sigma rule or 68-95-99.7 rule, is a rule in statistics which states for a normal distribution, almost all observed data will fall within three standard deviations of the mean. The rule predicts that 68% of the data will fall within the first standard deviation, 95% within the first two standard deviations, and 99.7% within the first three standard deviations. The empirical rule was applied to the Cq values for the individual samples, wet composited samples, and samples pooled after lysing by averaging the Cq values for each method of processing and adding one, two and three standard deviations. The average Cq values, standard deviation values, and averages plus one, two, and three standard deviations are found in Tables 46-50. The data shows that for samples lysed individually 68% with low levels of E. coli O157:H7 will generate a Cq call before cycle 34, 95% will call before cycle 36, and 99.7% will call before cycle 38. For samples either wet composited or pooled after lysing, 68% will have a Cq call before cycle 36, 95% before cycle 37, and 99.7% before cycle 38. Finally, the set containing the GFP strain demonstrated that the Cq calls for the GFP target were about the same as for the E. coli O157:H7 target regardless of lysing method. Also, the empirical rule generated the same results for the GFP target as the E. coli O157:H7 target, with 99.7% calling before cycle 38. The empirical rule demonstrates that even any outliers or anomalies should still test positive for both E. coli O157:H7 and Green Fluorescent Protein since 99.7% of the data had a less than 38 Cq value regardless of processing method. Also, because samples were spiked just above fractional levels, it would be expected that the Cq value for the empirical rule of the average plus three standard deviations would call earlier than 38 in routine samples.

The data outlined in this report confirms that the SIMUL-qPCR E. coli O157:H7 with GFP Assay is suitable for detecting E. coli O157:H7 in manual sampling devices for individual, wet composited, and pooled lysis samples. It also demonstrated that the assay could differentiate samples contaminated with a strain tagged with a Green Fluorescent Protein from those without the UV tag. Empirical rule analysis showed that the chances that an outlier would generate a negative result is extremely unlikely, regardless of if the samples were tested individually, wet composited, or the lysis pooled

TABLE 44
MSD E. coli O157:H7 Results

| | | 10 Hour | 10 Hour |
|---|---|---|---|
| MSD | 10 Hour | Wet | Pooled After |
| Sample | Individual | Composite | Lysing |
| Sample | Cq | Cq | Cq |
| 210701S001 | 27.316 | 27.463 | 29.144 |
| 210701S002 | 25.517 | 28.070 | 30.423 |
| 210701S003 | 27.887 | 29.669 | 31.813 |
| 210701S004 | 29.693 | 28.034 | 31.571 |
| 210701S005 | 25.105 | 27.566 | 29.636 |
| 210701S006 | 33.269 | 33.670 | 29.668 |
| 210701S007 | 30.873 | 32.543 | 30.647 |
| 210701S008 | 25.810 | 33.195 | 30.886 |
| 210701S009 | 26.400 | 32.108 | 33.204 |
| 210701S010 | 29.475 | 33.019 | 28.123 |
| 210701S011 | 23.774 | 31.022 | 28.431 |
| 210701S012 | 32.877 | 28.631 | 29.543 |
| 210701S013 | 26.211 | 27.920 | 21.439 |
| 210701S014 | 26.864 | 29.826 | 28.780 |
| 210701S015 | 34.097 | 28.939 | 32.047 |
| 210701S016 | 25.692 | 26.510 | 27.149 |
| 210701S017 | 25.416 | 28.507 | 28.290 |
| 210701S018 | 26.141 | 24.620 | 28.081 |
| 210701S019 | 26.141 | 25.911 | 27.997 |
| 210701S020 | 26.951 | 26.262 | 28.671 |
| 210701S021 | 33.086 | 35.017 | 33.916 |
| 210701S022 | 33.275 | 35.069 | 35.141 |
| 210701S023 | 34.446 | 35.738 | 35.133 |
| 210701S024 | 32.327 | 35.817 | 35.599 |
| 210701S025 | 33.474 | 34.987 | 34.556 |
| 210701S026 | 33.722 | 35.311 | 34.091 |
| 210701S027 | 33.593 | 36.480 | 35.876 |
| 210701S028 | 32.907 | 35.687 | 34.743 |
| 210701S029 | 32.929 | 35.222 | 35.327 |
| 210701S030 | 34.092 | 36.254 | 35.368 |
| 210701S031 | 33.586 | 36.866 | 35.271 |
| 210701S032 | 32.042 | 34.206 | 34.484 |
| 210701S033 | 31.498 | 34.563 | 33.855 |
| 210701S034 | 31.868 | 34.961 | 34.322 |
| 210701S035 | 32.739 | 35.968 | 35.072 |
| 210701S036 | 32.988 | 35.746 | 34.177 |
| 210701S037 | 31.916 | 35.932 | 34.514 |
| 210701S038 | 33.623 | 35.258 | 35.985 |
| 210701S039 | 34.743 | 35.266 | 35.624 |
| 210701S040 | 33.652 | 35.166 | 35.792 |
| 210701S041 | 32.508 | 32.719 | 32.508 |
| 210701S042 | 33.876 | 35.091 | 34.876 |
| 210701S043 | 32.519 | 34.432 | 32.519 |
| 210701S044 | 33.319 | 35.900 | 33.319 |
| 210701S045 | 30.391 | 34.720 | 33.391 |
| 210701S046 | 32.269 | 34.235 | 34.269 |
| 210701S047 | 29.493 | 31.658 | 31.493 |
| 210701S048 | 32.024 | 34.327 | 34.024 |
| 210701S049 | 28.800 | 32.585 | 31.800 |
| 210701S050 | 32.382 | 34.819 | 34.382 |
| 210701S051 | 30.348 | 32.593 | 31.348 |
| 210701S052 | 32.505 | 34.135 | 34.505 |
| 210701S053 | 30.191 | 33.198 | 32.191 |
| 210701S054 | 31.608 | 33.812 | 31.608 |
| 210701S055 | 32.855 | 34.994 | 34.855 |
| 210701S056 | 32.603 | 34.039 | 34.603 |
| 210701S057 | 31.468 | 32.744 | 32.468 |
| 210701S058 | 29.011 | 32.409 | 31.011 |
| 210701S059 | 32.680 | 34.628 | 33.680 |
| 210701S060 | 32.883 | 34.858 | 34.883 |
| 210701S061 | 28.767 | 32.217 | 32.811 |
| 210701S062 | 29.329 | 31.821 | 32.579 |
| 210701S063 | 34.780 | 35.989 | 34.275 |
| 210701S064 | 32.522 | 33.625 | 32.075 |
| 210701S065 | 32.938 | 33.484 | 34.190 |
| 210701S066 | 30.775 | 33.131 | 33.496 |
| 210701S067 | 28.528 | 31.747 | 32.311 |
| 210701S068 | 31.199 | 34.188 | 33.464 |
| 210701S069 | 27.766 | 30.164 | 31.196 |
| 210701S070 | 27.778 | 31.108 | 32.681 |
| 210701S071 | 28.022 | 31.761 | 30.690 |
| 210701S072 | 28.448 | 33.055 | 32.953 |
| 210701S073 | 32.487 | 35.467 | 35.203 |
| 210701S074 | 29.683 | 33.544 | 32.241 |
| 210701S075 | 29.885 | 32.118 | 33.167 |
| 210701S076 | 28.947 | 32.586 | 34.139 |
| 210701S077 | 29.271 | 33.605 | 33.834 |
| 210701S078 | 31.713 | 34.642 | 35.762 |
| 210701S079 | 32.130 | 33.818 | 34.033 |
| 210701S080 | 30.007 | 32.474 | 32.353 |
| 210701S081 | 28.647 | 31.489 | 32.479 |
| 210701S082 | 32.703 | 34.821 | 32.883 |
| 210701S083 | 32.512 | 34.572 | 35.379 |
| 210701S084 | 30.331 | 33.389 | 33.398 |
| 210701S085 | 30.603 | 33.178 | 33.427 |
| 210701S086 | 29.194 | 33.090 | 33.068 |
| 210701S087 | 29.279 | 31.103 | 32.638 |
| 210701S088 | 29.407 | 32.254 | 31.671 |
| 210701S089 | 30.218 | 33.785 | 35.164 |
| 210701S090 | 29.724 | 32.041 | 33.087 |
| 210701S091 | 33.606 | 34.149 | 35.038 |
| 210701S092 | 29.064 | 31.903 | 33.644 |
| 210701S093 | 28.786 | 32.372 | 32.085 |
| 210701S094 | 29.961 | 31.127 | 33.411 |
| 210701S095 | 32.902 | 34.276 | 34.304 |
| 210701S096 | 33.705 | 35.576 | 34.092 |
| 210701S097 | 31.213 | 31.720 | 33.786 |
| 210701S098 | 33.415 | 34.730 | 34.773 |
| 210701S099 | 26.571 | 30.556 | 31.887 |
| 210701S100 | 26.549 | 31.922 | 30.665 |

TABLE 45
GFP Results for Individual, Wet Composited,
and Pooled Lysis Samples

| | | 10 Hour | 10 Hour |
|---|---|---|---|
| MSD | 10 Hour | Wet | Pooled After |
| Sample | Individual | Composite | Lysing |
| Sample | Cq | Cq | Cq |
| 210701S001 | 32.245 | 35.857 | 34.579 |
| 210701S002 | 32.789 | 35.858 | 35.035 |
| 210701S003 | 31.930 | 34.289 | 34.786 |
| 210701S004 | 32.875 | 35.090 | 35.434 |
| 210701S005 | 32.547 | 35.144 | 34.164 |
| 210701S006 | 30.165 | 32.703 | 32.641 |
| 210701S007 | 34.567 | 35.868 | 35.420 |
| 210701S008 | 32.208 | 35.402 | 35.558 |
| 210701S009 | 32.790 | 33.909 | 35.100 |
| 210701S010 | 32.800 | 34.969 | 35.431 |
| 210701S011 | 31.445 | 33.970 | 33.614 |
| 210701S012 | 30.300 | 33.482 | 33.797 |
| 210701S013 | 33.319 | 35.329 | 35.090 |
| 210701S014 | 34.486 | 35.770 | 35.017 |
| 210701S015 | 32.229 | 35.033 | 35.445 |
| 210701S016 | 31.826 | 34.053 | 34.255 |
| 210701S017 | 33.440 | 35.624 | 35.262 |
| 210701S018 | 33.661 | 34.779 | 35.707 |
| 210701S019 | 34.429 | 36.489 | 35.044 |
| 210701S020 | 34.127 | 34.645 | 35.620 |
| 210701S021 | 0.000 | 0.000 | 0.000 |
| 210701S022 | 0.000 | 0.000 | 0.000 |
| 210701S023 | 0.000 | 0.000 | 0.000 |
| 210701S024 | 0.000 | 0.000 | 0.000 |
| 210701S025 | 0.000 | 0.000 | 0.000 |
| 210701S026 | 0.000 | 0.000 | 0.000 |
| 210701S027 | 0.000 | 0.000 | 0.000 |
| 210701S028 | 0.000 | 0.000 | 0.000 |
| 210701S029 | 0.000 | 0.000 | 0.000 |
| 210701S030 | 0.000 | 0.000 | 0.000 |
| 210701S031 | 0.000 | 0.000 | 0.000 |
| 210701S032 | 0.000 | 0.000 | 0.000 |
| 210701S033 | 0.000 | 0.000 | 0.000 |
| 210701S034 | 0.000 | 0.000 | 0.000 |
| 210701S035 | 0.000 | 0.000 | 0.000 |
| 210701S036 | 0.000 | 0.000 | 0.000 |
| 210701S037 | 0.000 | 0.000 | 0.000 |
| 210701S038 | 0.000 | 0.000 | 0.000 |
| 210701S039 | 0.000 | 0.000 | 0.000 |
| 210701S040 | 0.000 | 0.000 | 0.000 |
| 210701S041 | 0.000 | 0.000 | 0.000 |
| 210701S042 | 0.000 | 0.000 | 0.000 |
| 210701S043 | 0.000 | 0.000 | 0.000 |
| 210701S044 | 0.000 | 0.000 | 0.000 |
| 210701S045 | 0.000 | 0.000 | 0.000 |
| 210701S046 | 0.000 | 0.000 | 0.000 |
| 210701S047 | 0.000 | 0.000 | 0.000 |
| 210701S048 | 0.000 | 0.000 | 0.000 |
| 210701S049 | 0.000 | 0.000 | 0.000 |
| 210701S050 | 0.000 | 0.000 | 0.000 |
| 210701S051 | 0.000 | 0.000 | 0.000 |
| 210701S052 | 0.000 | 0.000 | 0.000 |
| 210701S053 | 0.000 | 0.000 | 0.000 |
| 210701S054 | 0.000 | 0.000 | 0.000 |
| 210701S055 | 0.000 | 0.000 | 0.000 |
| 210701S056 | 0.000 | 0.000 | 0.000 |
| 210701S057 | 0.000 | 0.000 | 0.000 |
| 210701S058 | 0.000 | 0.000 | 0.000 |
| 210701S059 | 0.000 | 0.000 | 0.000 |
| 210701S060 | 0.000 | 0.000 | 0.000 |
| 210701S061 | 0.000 | 0.000 | 0.000 |
| 210701S062 | 0.000 | 0.000 | 0.000 |
| 210701S063 | 0.000 | 0.000 | 0.000 |
| 210701S064 | 0.000 | 0.000 | 0.000 |
| 210701S065 | 0.000 | 0.000 | 0.000 |
| 210701S066 | 0.000 | 0.000 | 0.000 |
| 210701S067 | 0.000 | 0.000 | 0.000 |
| 210701S068 | 0.000 | 0.000 | 0.000 |
| 210701S069 | 0.000 | 0.000 | 0.000 |
| 210701S070 | 0.000 | 0.000 | 0.000 |
| 210701S071 | 0.000 | 0.000 | 0.000 |
| 210701S072 | 0.000 | 0.000 | 0.000 |
| 210701S073 | 0.000 | 0.000 | 0.000 |
| 210701S074 | 0.000 | 0.000 | 0.000 |
| 210701S075 | 0.000 | 0.000 | 0.000 |
| 210701S076 | 0.000 | 0.000 | 0.000 |
| 210701S077 | 0.000 | 0.000 | 0.000 |
| 210701S078 | 0.000 | 0.000 | 0.000 |
| 210701S079 | 0.000 | 0.000 | 0.000 |
| 210701S080 | 0.000 | 0.000 | 0.000 |
| 210701S081 | 0.000 | 0.000 | 0.000 |
| 210701S082 | 0.000 | 0.000 | 0.000 |
| 210701S083 | 0.000 | 0.000 | 0.000 |
| 210701S084 | 0.000 | 0.000 | 0.000 |
| 210701S085 | 0.000 | 0.000 | 0.000 |
| 210701S086 | 0.000 | 0.000 | 0.000 |
| 210701S087 | 0.000 | 0.000 | 0.000 |
| 210701S088 | 0.000 | 0.000 | 0.000 |
| 210701S089 | 0.000 | 0.000 | 0.000 |
| 210701S090 | 0.000 | 0.000 | 0.000 |
| 210701S091 | 0.000 | 0.000 | 0.000 |
| 210701S092 | 0.000 | 0.000 | 0.000 |
| 210701S093 | 0.000 | 0.000 | 0.000 |
| 210701S094 | 0.000 | 0.000 | 0.000 |
| 210701S095 | 0.000 | 0.000 | 0.000 |
| 210701S096 | 0.000 | 0.000 | 0.000 |
| 210701S097 | 0.000 | 0.000 | 0.000 |
| 210701S098 | 0.000 | 0.000 | 0.000 |
| 210701S099 | 0.000 | 0.000 | 0.000 |
| 210701S100 | 0.000 | 0.000 | 0.000 |

TABLE 46
Empirical Data Samples Inoculated with E. coli O157:H7 FDA ESC1177

| Samples 1-20 | | 10 Hour | 10 Hour | | | 10 Hour | 10 Hour |
|---|---|---|---|---|---|---|---|
| (E. coli O157:H7 | 10 Hour | Wet | Pooled After | Samples 1-20 | 10 Hour | Wet | Pooled After |
| Target) | Individual | Composite | Lysing | (GFP Target) | Individual | Composite | Lysing |
| AVG | 32.664 | 34.900 | 34.950 | AVG | 32.709 | 34.913 | 34.850 |
| 1STDEV | 1.227 | 0.904 | 0.902 | 1STDEV | 1.244 | 0.945 | 0.794 |
| 2STDEV | 2.454 | 1.809 | 1.803 | 2STDEV | 2.489 | 1.891 | 1.589 |
| 3STDEV | 3.681 | 2.713 | 2.705 | 3STDEV | 3.733 | 2.836 | 2.383 |
| 1SIGMA_Cq | 33.891 | 35.805 | 35.851 | 1SIGMA_Cq | 33.953 | 35.858 | 35.644 |
| 2SIGMA_Cq | 35.118 | 36.709 | 36.753 | 2SIGMA_Cq | 35.198 | 36.804 | 36.439 |
| 3SIGMA_Cq | 36.345 | 37.614 | 37.655 | 3SIGMA_Cq | 36.442 | 37.749 | 37.233 |

TABLE 47
Empirical Data for Samples Inoculated with E. coli O157:H7 ATCC 43886

| Samples 21-40 | | 10 Hour | 10 Hour | Samples 21- | | 10 Hour | 10 Hour |
|---|---|---|---|---|---|---|---|
| (E. coli O157:H7 | 10 Hour | Wet | Pooled After | 40 (GFP | 10 Hour | Wet | Pooled After |
| Target) | Individual | Composite | Lysing | Target) | Individual | Composite | Lysing |
| AVG | 33.125 | 35.476 | 34.942 | AVG | 0.000 | 0.000 | 0.000 |
| 1STDEV | 0.874 | 0.645 | 0.674 | 1STDEV | 0.000 | 0.000 | 0.000 |
| 2STDEV | 1.748 | 1.290 | 1.347 | 2STDEV | 0.000 | 0.000 | 0.000 |
| 3STDEV | 2.622 | 1.935 | 2.021 | 3STDEV | 0.000 | 0.000 | 0.000 |
| 1SIGMA_Cq | 33.999 | 36.121 | 35.616 | 1SIGMA_Cq | 0.000 | 0.000 | 0.000 |
| 2SIGMA_Cq | 34.873 | 36.766 | 36.289 | 2SIGMA_Cq | 0.000 | 0.000 | 0.000 |
| 3SIGMA_Cq | 35.747 | 37.411 | 36.963 | 3SIGMA_Cq | 0.000 | 0.000 | 0.000 |

TABLE 48
Empirical Data for Samples Inoculated with E. coli O157:H7 NR-22047

| Samples 41-60 | | 10 Hour | 10 Hour | Samples 41- | | 10 Hour | 10 Hour |
|---|---|---|---|---|---|---|---|
| (E. coli O157:H7 | 10 Hour | Wet | Pooled After | 60 (GFP | 10 Hour | Wet | Pooled After |
| Target) | Individual | Composite | Lysing | Target) | Individual | Composite | Lysing |
| AVG | 31.687 | 33.895 | 33.187 | AVG | 0.000 | 0.000 | 0.000 |
| 1STDEV | 1.471 | 1.127 | 1.331 | 1STDEV | 0.000 | 0.000 | 0.000 |
| 2STDEV | 2.942 | 2.255 | 2.663 | 2STDEV | 0.000 | 0.000 | 0.000 |
| 3STDEV | 4.413 | 3.382 | 3.994 | 3STDEV | 0.000 | 0.000 | 0.000 |
| 1SIGMA_Cq | 33.158 | 35.022 | 34.518 | 1SIGMA_Cq | 0.000 | 0.000 | 0.000 |
| 2SIGMA_Cq | 34.629 | 36.149 | 35.849 | 2SIGMA_Cq | 0.000 | 0.000 | 0.000 |
| 3SIGMA_Cq | 36.100 | 37.277 | 37.181 | 3SIGMA_Cq | 0.000 | 0.000 | 0.000 |

TABLE 49
Empirical Data for Samples Inoculated with E. coli O157:H7 ATCC 700728

| Samples 61-80 | | 10 Hour | 10 Hour | Samples 61- | | 10 Hour | 10 Hour |
|---|---|---|---|---|---|---|---|
| (E. coli O157:H7 | 10 Hour | Wet | Pooled After | 80 (GFP | 10 Hour | Wet | Pooled After |
| Target) | Individual | Composite | Lysing | Target) | Individual | Composite | Lysing |
| AVG | 30.249 | 33.027 | 33.173 | AVG | 0.000 | 0.000 | 0.000 |
| 1STDEV | 1.979 | 1.433 | 1.250 | 1STDEV | 0.000 | 0.000 | 0.000 |
| 2STDEV | 3.959 | 2.867 | 2.501 | 2STDEV | 0.000 | 0.000 | 0.000 |
| 3STDEV | 5.938 | 4.300 | 3.751 | 3STDEV | 0.000 | 0.000 | 0.000 |
| 1SIGMA_Cq | 32.228 | 34.461 | 34.423 | 1SIGMA_Cq | 0.000 | 0.000 | 0.000 |
| 2SIGMA_Cq | 34.207 | 35.894 | 35.674 | 2SIGMA_Cq | 0.000 | 0.000 | 0.000 |
| 3SIGMA_Cq | 36.187 | 37.327 | 36.924 | 3SIGMA_Cq | 0.000 | 0.000 | 0.000 |

TABLE 50
Empirical Data for Samples Inoculated with E. coli O157:H7 NCTC 12900

| Samples 81-100 | | 10 Hour | 10 Hour | Samples 81- | | 10 Hour | 10 Hour |
|---|---|---|---|---|---|---|---|
| (E. coli O157:H7 | 10 Hour | Wet | Pooled After | 100 (GFP | 10 Hour | Wet | Pooled After |
| Target) | Individual | Composite | Lysing | Target) | Individual | Composite | Lysing |
| AVG | 30.420 | 32.903 | 33.344 | AVG | 0.000 | 0.000 | 0.000 |
| 1STDEV | 2.156 | 1.455 | 1.245 | 1STDEV | 0.000 | 0.000 | 0.000 |
| 2STDEV | 4.313 | 2.910 | 2.489 | 2STDEV | 0.000 | 0.000 | 0.000 |
| 3STDEV | 6.469 | 4.365 | 3.734 | 3STDEV | 0.000 | 0.000 | 0.000 |
| 1SIGMA_Cq | 32.576 | 34.358 | 34.589 | 1SIGMA_Cq | 0.000 | 0.000 | 0.000 |
| 2SIGMA_Cq | 34.732 | 35.813 | 35.833 | 2SIGMA_Cq | 0.000 | 0.000 | 0.000 |
| 3SIGMA_Cq | 36.888 | 37.268 | 37.078 | 3SIGMA_Cq | 0.000 | 0.000 | 0.000 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for detecting cross-contamination and the presence or absence of a target bacterial species in a test sample, said method comprising:
providing (i) a test sample potentially comprising a target bacteria and (ii) a control sample, wherein the control sample comprises a control bacterial strain engineered with a reporter molecule;
lysing the test sample and the control sample to generate a test sample lysate and a control sample lysate;
contacting each of the test sample lysate and the control sample lysate with an assay mixture comprising one or more bacterial detection PCR primer sets, and a control detection PCR primer set, thereby generating one or more assay samples capable of amplifying a target;
detecting the presence or absence of an amplified target PCR product and/or an amplified control PCR product in each of the one or more assay samples, thereby determining the presence or absence of the target bacterial species and/or the control bacterial strain in the samples simultaneously; and
determining if cross-contamination of the test sample occurred by detecting if the amplified control PCR product is present in the test sample.

2. The method of claim 1, further comprising:
incubating the test sample and the control sample, separately, prior to said lysing, in an enrichment and/or selective media to enrich target bacteria in the test sample, if present, and the bacterial strain in the control sample.

3. The method of claim 1, wherein said detecting the presence or absence of an amplified target PCR product and/or an amplified control PCR product comprises determining if a Cq value is present or absent.

4. The method of claim 1, wherein the Cq value of the amplified control PCR product occurs at about the same time or prior to the Cq value of the amplified test sample, if target bacteria are present in the test sample.

5. The method of claim 1, wherein the test sample is from a food.

6. (canceled)

7. The method of claim 1, wherein the test sample is an environmental sample.

8-9. (canceled)

10. The method of claim 1, wherein the test sample is from a plant.

11. (canceled)

12. The method of claim 1, further comprising:
pre-treating the test sample and/or the control sample to remove nucleic acids not associated with intact cells, thereby generating a pre-treated sample test sample and/or pre-treated control sample before said lysing.

13. (canceled)

14. The method of claim 1, wherein said detecting is carried out with fluorescence imaging.

15. The method of claim 1, wherein the assay mixture further comprises an internal amplification control PCR primer set.

16. (canceled)

17. The method of claim 1, wherein the target bacterial species is one or more bacteria from the genus Salmonella, Listeria, Escherichia, Campylobacter, Vibrio, or Cronobacter, or any combination thereof.

18. The method of claim 1, wherein the assay mixture comprises (i) one or more PCR primer sets capable of detecting Salmonella and (ii) one or more PCR primer sets capable of detecting the reporter molecule.

19-23. (canceled)

24. The method of claim 18, wherein the one or more PCR primer sets comprise: Salmonella 1 (SEQ ID NOs: 1-2), Salmonella 2 (SEQ ID NOs:4-5), and Green Fluorescent Protein (GFP) (SEQ ID NOs:7-8).

25. (canceled)

26. The method of claim 1, wherein the target bacterial species is a Listeria species.

27. The method of claim 26, wherein the assay mixture comprises (i) one or more PCR primer sets capable of detecting Listeria and (ii) one or more PCR primer sets capable of detecting the reporter molecule.

28-30. (canceled)

31. The method of claim 27, wherein the one or more PCR primer sets comprise: Listera spp. (SEQ ID NOs: 10-11), Listeria monocytogenes (SEQ ID NOs:13-14), and Green Fluorescent Protein (GFP) (SEQ ID NOs:7-8).

32. (canceled)

33. The method of claim 1, wherein the target bacterial species is an E. coli species.

34. The method of claim 33, wherein the assay mixture comprises (i) one or more PCR primer sets capable of detecting E. coli and (ii) one or more PCR primer sets capable of detecting the reporter molecule.

35-38. (canceled)

39. The method of claim 34, wherein the one or more PCR primer sets comprises one or more of E. coli O157:H7 (SEQ ID NOs: 16-17), Shiga toxin gene(stx) 1 (SEQ ID NOs: 19-20), Shiga toxin gene(stx) 2 (SEQ ID NOs:22-23), Intimin gene (eae) (SEQ ID NOs: 25-26), E. coli O103 (SEQ ID NOs:28-29) and E. coli O26 (SEQ ID NOs:31-32), E. coli O145 (SEQ ID NOs:34-35), E. coli O111 (SEQ ID NOs:37-38), E. coli O45 (SEQ ID NOs:40-41), E. coli O121 (SEQ ID NOs:43-44), Green Fluorescent Protein (GFP) (SEQ ID NOs:7-8), IAC (SEQ ID NOs: 46-47), or any combination thereof.

40. (canceled)

41. A kit comprising:
one or more PCR primer sets capable of detecting Salmonella, Listeria, or E. coli species and
one or more PCR primer sets capable of detecting a control bacterial strain engineered with a reporter molecule.

42. (canceled)


SEQUENCE LISTING (49 sequences)

SEQ ID NO: 1 - DNA, 20 bp, synthetic construct
  source 1..20: mol_type = other DNA
  misc_feature 1..20: note = Salmonella 1 Forward Primer
agcgtactggaaagggaaag

SEQ ID NO: 2 - DNA, 24 bp, synthetic construct
  source 1..24: mol_type = other DNA
  misc_feature 1..24: note = Salmonella 1 Reverse Primer
ataccgccaataaagttcacaaag

SEQ ID NO: 3 - DNA, 26 bp, synthetic construct
  source 1..26: mol_type = other DNA
  misc_feature 1..26: note = Salmonella 1 Probe
cgtcacctttgataaacttcatcgca

SEQ ID NO: 4 - DNA, 21 bp, synthetic construct
  source 1..21: mol_type = other DNA
  misc_feature 1..21: note = Salmonella 2 Forward Primer
agcaaacccaggtgaaactga

SEQ ID NO: 5 - DNA, 24 bp, synthetic construct
  source 1..24: mol_type = other DNA
  misc_feature 1..24: note = Salmonella 2 Reverse Primer
aggcttaccgtcaaaagttaacgt

SEQ ID NO: 6 - DNA, 23 bp, synthetic construct
  source 1..23: mol_type = other DNA
  misc_feature 1..23: note = Salmonella 2 Probe
tcactggcacaggcattccatcg

SEQ ID NO: 7 - DNA, 29 bp, synthetic construct
  source 1..29: mol_type = other DNA
  misc_feature 1..29: note = GFP Forward Primer
cattgaagatggatccgttcaactagcag

SEQ ID NO: 8 - DNA, 27 bp, synthetic construct
  source 1..27: mol_type = other DNA
  misc_feature 1..27: note = GFP Reverse Primer
atcccagcagcagttacaaactcaaga

SEQ ID NO: 9 - DNA, 30 bp, synthetic construct
  source 1..30: mol_type = other DNA
  misc_feature 1..30: note = GFP Probe
aggacagggccatcgccaattggagtattt

SEQ ID NO: 10 - DNA, 20 bp, synthetic construct
  source 1..20: mol_type = other DNA
  misc_feature 1..20: note = Listeria spp. Forward Primer
aggatagggaatcgcacgaa

SEQ ID NO: 11 - DNA, 18 bp, synthetic construct
  source 1..18: mol_type = other DNA
  misc_feature 1..18: note = Listeria spp. Reverse Primer
ttcgcgagaagcggattt

SEQ ID NO: 12 - DNA, 23 bp, synthetic construct
  source 1..23: mol_type = other DNA
  misc_feature 1..23: note = Listeria spp. Probe
tctcacactcactgcttggacgc

SEQ ID NO: 13 - DNA, 19 bp, synthetic construct
  source 1..19: mol_type = other DNA
  misc_feature 1..19: note = Listeria monocytogenes Forward Primer
catggcaccaccagcatct

SEQ ID NO: 14 - DNA, 20 bp, synthetic construct
  source 1..20: mol_type = other DNA
  misc_feature 1..20: note = Listeria monocytogenes Reverse Primer
atccgcgtgtttcttttcga

SEQ ID NO: 15 - DNA, 23 bp, synthetic construct
  source 1..23: mol_type = other DNA
  misc_feature 1..23: note = Listeria monocytogenes Probe
cgcctgcaagtcctaagacgcca

SEQ ID NO: 16 - DNA, 21 bp, synthetic construct
  source 1..21: mol_type = other DNA
  misc_feature 1..21: note = E. coli O157:H7 Forward Primer
tattccgcgatgcttgttttt

SEQ ID NO: 17 - DNA, 24 bp, synthetic construct
  source 1..24: mol_type = other DNA
  misc_feature 1..24: note = E. coli O157:H7 Reverse Primer
attatctcaccagcaaactggcgg

SEQ ID NO: 18 - DNA, 13 bp, synthetic construct
  source 1..13: mol_type = other DNA
  misc_feature 1..13: note = E. coli O157:H7 Probe
ccgcaatctttcc

SEQ ID NO: 19 - DNA, 26 bp, synthetic construct
  source 1..26: mol_type = other DNA
  misc_feature 1..26: note = Shiga Toxin 1 (stx) Forward Primer
gtggcattaatactgaattgtcatca

SEQ ID NO: 20 - DNA, 21 bp, synthetic construct
  source 1..21: mol_type = other DNA
  misc_feature 1..21: note = Shiga Toxin 1 (stx) Reverse Primer
gcgtaatcccacggactcttc

SEQ ID NO: 21 - DNA, 31 bp, synthetic construct
  source 1..31: mol_type = other DNA
  misc_feature 1..31: note = Shiga Toxin 1 (stx) Probe
tgatgagtttccttctatgtgtccggcagat

SEQ ID NO: 22 - DNA, 24 bp, synthetic construct
  source 1..24: mol_type = other DNA
  misc_feature 1..24: note = Shiga Toxin 2 (stx) Forward Primer
gatgtttatggcggttttatttgc

SEQ ID NO: 23 - DNA, 26 bp, synthetic construct
  source 1..26: mol_type = other DNA
  misc_feature 1..26: note = Shiga Toxin 2 (stx) Reverse Primer
tggaaaactcaattttacctttagca

SEQ ID NO: 24 - DNA, 25 bp, synthetic construct
  source 1..25: mol_type = other DNA
  misc_feature 1..25: note = Shiga Toxin 2 (stx) Probe
tctgttaatgcaatggcggcggatt

SEQ ID NO: 25 - DNA, 19 bp, synthetic construct
  source 1..19: mol_type = other DNA
  misc_feature 1..19: note = Intimin (eae) Forward Primer
ccgattcctctggtgacga

SEQ ID NO: 26 - DNA, 24 bp, synthetic construct
  source 1..24: mol_type = other DNA
  misc_feature 1..24: note = Intimin (eae) Reverse Primer
ccacggtttatcaaactgataacg

SEQ ID NO: 27 - DNA, 17 bp, synthetic construct
  source 1..17: mol_type = other DNA
  misc_feature 1..17: note = Intimin (eae) Probe
cgtcatggtacgggtaa

SEQ ID NO: 28 - DNA, 20 bp, synthetic construct
  source 1..20: mol_type = other DNA
  misc_feature 1..20: note = E. coli O103 Forward Primer
ttggagcgttaactggacct

SEQ ID NO: 29 - DNA, 24 bp, synthetic construct
  source 1..24: mol_type = other DNA
  misc_feature 1..24: note = E. coli O103 Reverse Primer
atattcgctatatcttcttgcggc

SEQ ID NO: 30 - DNA, 28 bp, synthetic construct
  source 1..28: mol_type = other DNA
  misc_feature 1..28: note = E. coli O103 Probe
aggcttatctggctgttcttactacggc

SEQ ID NO: 31 - DNA, 22 bp, synthetic construct
  source 1..22: mol_type = other DNA
  misc_feature 1..2: note = E. coli O26 Forward Primer
gtatcgctgaaattagaagcgc

SEQ ID NO: 32 - DNA, 21 bp, synthetic construct
  source 1..21: mol_type = other DNA
  misc_feature 1..21: note = E. coli O26 Reverse Primer
agttgaaacacccgtaatggc

SEQ ID NO: 33 - DNA, 28 bp, synthetic construct
  source 1..28: mol_type = other DNA
  misc_feature 1..28: note = E. coli O26 Probe
tggttcggttggattgtccataagaggg

SEQ ID NO: 34 - DNA, 19 bp, synthetic construct
  source 1..19: mol_type = other DNA
  misc_feature 1..19: note = E. coli O145 Forward Primer
aaactgggattggacgtgg

SEQ ID NO: 35 - DNA, 19 bp, synthetic construct
  source 1..19: mol_type = other DNA
  misc_feature 1..19: note = E. coli O145 Reverse Primer
cccaaaacttctaggcccg

SEQ ID NO: 36 - DNA, 25 bp, synthetic construct
  source 1..25: mol_type = other DNA
  misc_feature 1..25: note = E. coli O145 Probe
tgctaattgcagcccttgcactacg

SEQ ID NO: 37 - DNA, 21 bp, synthetic construct
  source 1..21: mol_type = other DNA
  misc_feature 1..21: note = E. coli O111 Forward Primer
tgttccaggtggtaggattcg

SEQ ID NO: 38 - DNA, 21 bp, synthetic construct
  source 1..21: mol_type = other DNA
  misc_feature 1..21: note = E. coli O111 Reverse Primer
tcacgatgttgatcatctggg

SEQ ID NO: 39 - DNA, 29 bp, synthetic construct
  source 1..29: mol_type = other DNA
  misc_feature 1..29: note = E. coli O111 Probe
tgaaggcgaggcaacacattatatagtgc

SEQ ID NO: 40 - DNA, 18 bp, synthetic construct
  source 1..18: mol_type = other DNA
  misc_feature 1..18: note = E. coli O45 Forward Primer
cgttgtgcatggtggcat

SEQ ID NO: 41 - DNA, 22 bp, synthetic construct
  source 1..22: mol_type = other DNA
  misc_feature 1..22: note = E. coli O45 Reverse Primer
tggccaaaccaactatgaactg

SEQ ID NO: 42 - DNA, 26 bp, synthetic construct
  source 1..26: mol_type = other DNA
  misc_feature 1..26: note = E. coli O45 Probe
attttttgctgcaagtgggctgtcca

SEQ ID NO: 43 - DNA, 22 bp, synthetic construct
  source 1..22: mol_type = other DNA
  misc_feature 1..22: note = E. coli O121 Forward Primer
aggcgctgtttggtctcttaga

SEQ ID NO: 44 - DNA, 20 bp, synthetic construct
  source 1..20: mol_type = other DNA
  misc_feature 1..20: note = E. coli O121 Reverse Primer
gaaccgaaatgatgggtgct

SEQ ID NO: 45 - DNA, 24 bp, synthetic construct
  source 1..24: mol_type = other DNA
  misc_feature 1..24: note = E. coli O121 Probe
cgctatcatggcgggacaatgaca

SEQ ID NO: 46 - DNA, 23 bp, synthetic construct
  source 1..23: mol_type = other DNA
  misc_feature 1..23: note = IAC Forward Primer
ctaaccttcgtgatgagcaatcg

SEQ ID NO: 47 - DNA, 22 bp, synthetic construct
  source 1..22: mol_type = other DNA
  misc_feature 1..22: note = IAC Reverse Primer
gatcagctacgtgaggtcctac

SEQ ID NO: 48 - DNA, 26 bp, synthetic construct
  source 1..26: mol_type = other DNA
  misc_feature 1..26: note = IAC Probe
agctagtcgatgcactccagtcctcc

SEQ ID NO: 49 - DNA, 720 bp, synthetic construct
  source 1..720: mol_type = other DNA
  misc_feature 1..720: note = green fluorescent protein
atggctagcaaaggagaagaactggtacctggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa